(19) United States
(12) Patent Application Publication
(10) Pub. No.: US 2024/0218356 A1
(43) Pub. Date: Jul. 4, 2024

(54) METHODS FOR PREPARING A LIBRARY OF PLANT DISEASE RESISTANCE GENES FOR FUNCTIONAL TESTING FOR DISEASE RESISTANCE

(71) Applicant: Two Blades Foundation, Evanston, IL (US)

(72) Inventors: Matthew James Moscou, Norwich (GB); Hendrikus Pieter Van Esse, Norwich (GB)

(73) Assignee: Two Blades Foundation, Evanston, IL (US)

(21) Appl. No.: 18/558,574

(22) PCT Filed: May 11, 2022
(86) PCT No.: PCT/US2022/028686

§ 371 Date: Nov. 2, 2023

Related U.S. Application Data
(60) Provisional application No. 63/186,986, filed on May 11, 2021.

(51) Int. Cl. C12N 15/10; C12N 15/82

(52) U.S. Cl. CPC C12N 15/1093; C12N 15/8279

(57) ABSTRACT

Methods are provided for preparing a library of candidate plant disease resistance (R) genes against a plant pathogen of interest. The methods involve selecting from each of one or more plants of interest a subpopulation of highly expressed nucleotide-binding leucine rich repeat genes (NLRs) from among the population of NLRs that are constitutively expressed in an organ or other part of the one or more plants to produce a library of candidate R genes. Further provided are related methods for identifying R genes against a plant pathogen of interest using a library of candidate R genes and compositions comprising the identified R genes.

### CROSS-REFERENCE TO RELATED APPLICATION

This application claims the benefit of U.S. Provisional Patent Application No. 63/186,986 filed May 11, 2021, which is hereby incorporated herein in its entirety by reference.

### REFERENCE TO A SEQUENCE LISTING SUBMITTED AS A TEXT FILE

The official copy of the sequence listing is submitted electronically via EFS-Web as an ASCII formatted sequence listing with a file named 070294-0201SEQLST.TXT, created on May 9, 2022 and having a size of 1.88 megabytes, and is filed concurrently with the specification. The sequence listing contained in this ASCII formatted document is part of the specification and is herein incorporated by reference in its entirety.

### FIELD OF THE INVENTION

The present invention relates to the fields of plant disease resistance and crop plant improvement, particularly to methods that are useful for the identification of plant disease resistance genes against plant pathogens in a crop plant of interest.

### BACKGROUND OF THE INVENTION

Plant disease causes significant yield losses in agriculture. Among the most damaging diseases are filamentous plant pathogens, most notably fungi and oomycetes. These pests are key challenges for growers and cause significant management costs. The most cost effective and environmentally friendly way of managing these diseases is the use of resistance genes that can often be found in wild relatives of crops or even unrelated plant species. Wild relatives of domesticated crops contain many useful disease resistance (R) genes. Introducing this natural resistance is an elegant way of managing disease. However, traditional methods for introducing R genes typically involve long breeding trajectories to avoid “linkage drag,” i.e. the simultaneous introduction of deleterious traits with the R gene. Furthermore, R genes tend to be overcome by the pathogen within a few seasons when deployed one at a time.

An approach to preventing a pathogen from quickly overcoming the resistance provided by a single R gene is to deploy simultaneously multiple R genes against the pathogen in a crop plant. Although such an approach can be accomplished by traditional plant breeding methods, the multiple R genes would very likely be found scattered throughout the genome of the plant of interest, making the combination of the multiple R genes into a single plant extremely laborious and time consuming. In addition, this task becomes more challenging if multiple pathogens are critical to be controlled to ensure a successful harvest. Alternatively, transgenic approaches can be used to rapidly deploy multiple R genes into a single crop plant. The multiple R genes can be introduced into a single crop plant as transgenes via routine genetic engineering techniques. Preferably, the multiple R genes would be introduced as a single, multi-transgene cassette that segregates as a single locus to facilitate the rapid transfer of the multiple R genes to breeding lines and crop plant cultivars.

Traditional map-based cloning of R genes remains challenging despite great strides in sequencing technology and biological insights; large tracts of plant genomes are inaccessible to map-based genetics due to lack of recombination. Most R genes belong to a structural class of genes that encode nucleotide-binding leucine-rich repeat (NLR) proteins. NLRs (i.e. genes encoding NLR proteins) tend to reside in complex clusters in plant genomes, and many hundreds of NLRs populate a typical plant genome. Thus, a scientist using a traditional map-based cloning method, therefore frequently delimits a map interval containing multiple NLRs and must find out which confers the resistance of interest. Recently, a new method, which is known as Resistance Gene Enrichment Sequencing (RenSeq), has been reported that allows rapid scrutiny of all the NLRs within a plant. (Jupe et al., 2013, Plant J. 76(3):530-44). While the RenSeq method can be used to the rapidly identify NLRs sequences in a plant, the RenSeq method does not allow for the identification of an NLR gene that is specific to a plant disease of interest in the absence of additional genetic approaches.

More recently, MutRenSeq was developed to allow for the identification of an R gene that is specific to a plant disease of interest in the absence of additional map-based genetics (Steuernagel et al., 2017, Methods Mol. Biol. 1659:215-229). While MutRenSeq has proven to useful for the identification of NLR genes from plants comprising resistance to plant disease of interest, the method depends on producing a susceptible plant by mutagenizing a plant that is resistance to the disease of interest and then comparing the nucleotide sequences of the NLR genes from the resistant plant with the susceptible plant to identify the NLR gene that was modified in the susceptible plant. However, because the production of such a susceptible plant can be challenging, new approaches for identifying an R gene for a disease of interest that limit the number of potential candidate R genes and that do not depend on the production of a susceptible plant by mutagenizing a plant harbouring an R gene for the disease of interest.

### BRIEF SUMMARY OF THE INVENTION

The present invention provides methods for preparing a library of candidate plant disease resistance (R) genes, particularly R genes encoding nucleotide-binding leucine-rich repeat (NLR) proteins, against one or more plant pathogens of interest. The methods comprise selecting from each of one or more plants of interest, a subpopulation of highly expressed nucleotide-binding leucine rich repeat genes (NLRs) from among a population of constitutively expressed NLRs in an organ or other part of the one or more plants, so as to produce a library of candidate R genes. The subpopulation of highly expressed NLRs comprises NLRs that are highly expressed, constitutively in an organ or other part of the plant in the absence of the plant or any organ or other part thereof being contacted with or otherwise exposed to one or more plant pathogens of interest. Such highly expressed NLRs are those NLRs that comprise a relative expression level in the organ or other part of the plant that is greater than the relative expression levels in the organ or other part of the plant of at least about 65% of the NLRs in the population of constitutively expressed NLRs in the organ or other part of the plant.

The present invention further provides methods for identifying an R gene that is capable of conferring to a plant resistance to a plant pathogen of interest. Such methods comprise contacting a transgenic plant comprising a candidate R gene or a collection of transgenic plants each comprising a candidate R gene with the plant pathogen of interest. The candidate R genes are from a library of candidate R genes produced as describe above. Each of such transgenic plants can be produced by transforming a host plant with a candidate R gene. The host plant is a host (i.e. susceptible plant) for the plant pathogen of interest. That is, the plant pathogen is capable of causing plant disease symptoms on the host plant under suitable environment conditions. The methods further comprise contacting the transgenic plant(s) with, or otherwise exposing the transgenic plant(s) to, the plant pathogen of interest under environmental conditions suitable for the development of disease symptoms on a susceptible plant and determining if a transgenic plant displays enhanced resistance to the plant pathogen of interest when compared to a control host plant that does not comprise the candidate NLR gene. Candidate NLR genes that confer to such a transgenic plant resistance to plant disease symptoms caused by the plant pathogen of interest are identified as functional NLR genes.

Further provided are libraries comprising candidate NLR genes, collections of transgenic plants comprising candidate NLR genes, nucleic molecules comprising one or more NLR genes identified according the methods of the present invention and plants, plant cells, and other host cells comprising one or more such NLR genes.

### BRIEF DESCRIPTION OF THE DRAWINGS

FIG. 1 is a graphical representation of transcript abundance of NLRs from the de novo assembled transcriptome of barley (Hordeum vulgare) accession Golden Promise. Transcript abundance was estimated from self-aligned RNAseq data measured in transcripts per million (TPM). The expression of two functional resistance genes, Rps6 and Rps7.a (Mla8), to wheat stripe rust (Puccinia striiformis f. sp. tritici) is shown.

FIG. 2 is a graphical representation of transcript abundance of NLRs from the de novo assembled transcriptome of barley (Hordeum vulgare) accession CI 16147. Transcript abundance was estimated from self-aligned RNAseq data measured in transcripts per million (TPM). The expression of the functional resistance gene Rps7.b (Mla7) to wheat stripe rust (Puccinia striiformis f. sp. tritici) is shown.

FIG. 3 is a graphical representation of transcript abundance of NLRs from the de novo assembled transcriptome of barley (Hordeum vulgare) accession CI 16153. Transcript abundance was estimated from self-aligned RNAseq data measured in transcripts per million (TPM). The expression of the functional resistance gene Rps7.b (Mla7) to wheat stripe rust (Puccinia striiformis f. sp. tritici) is shown.

FIG. 4 is a graphical representation of transcript abundance of NLRs from the de novo assembled transcriptome of pigeon pea (Cajanus cajan) accession G119-99. Transcript abundance was estimated from self-aligned RNAseq data measured in transcripts per million (TPM). The expression of the functional resistance gene Rpp1 to Asian soybean rust (Phakopsora pachyrhizi) is shown.

FIG. 5 is a graphical representation of transcript abundance of NLRs from the de novo assembled transcriptome of Arabidopsis thaliana accession Col-0. Transcript abundance was estimated from self-aligned RNAseq data measured in transcripts per million (TPM). The expression of the functional resistance genes RPP1, RPP4, RPP5, RPP7, and RPP8 to downy mildew (Hyaloperonospora arabidopsidis), WRR4 to white rust (Albugo candida), and ZAR1 to Pseudomonas syringae are shown.

FIG. 6 is a graphical representation of transcript abundance of NLRs from the de novo assembled transcriptome of Aegilops tauschii accession KU2025. Transcript abundance was estimated from self-aligned RNAseq data measured in transcripts per million (TPM). The expression of the functional resistance gene Sr46 to wheat stem rust (Puccinia graminis f. sp. tritici) is shown.

FIG. 7 is a graphical representation of transcript abundance of NLRs from the de novo assembled transcriptome of Aegilops tauschii accession KU2075. Transcript abundance was estimated from self-aligned RNAseq data measured in transcripts per million (TPM). The expression of the functional resistance gene Sr46 to wheat stem rust (Puccinia graminis f. sp. tritici) is shown.

FIG. 8 is a graphical representation of transcript abundance of NLRs from the de novo assembled transcriptome of Aegilops tauschii accession KU2078. Transcript abundance was estimated from self-aligned RNAseq data measured in transcripts per million (TPM). The expression of the functional resistance gene Sr46 and SrTA1662 to wheat stem rust (Puccinia graminis f. sp. tritici) is shown.

FIG. 9 is a graphical representation of transcript abundance of NLRs from the de novo assembled transcriptome of Aegilops tauschii accession KU2093. Transcript abundance was estimated from self-aligned RNAseq data measured in transcripts per million (TPM). The expression of the functional resistance gene Sr46 to wheat stem rust (Puccinia graminis f. sp. tritici) is shown.

FIG. 10 is a graphical representation of transcript abundance of NLRs from the de novo assembled transcriptome of Aegilops tauschii accession KU2124. Transcript abundance was estimated from self-aligned RNAseq data measured in transcripts per million (TPM). The expression of the functional resistance gene Sr45 to wheat stem rust (Puccinia graminis f. sp. tritici) is shown.

FIG. 11 is a graphical representation of transcript abundance of NLRs from the de novo assembled transcriptome of Aegilops tauschii accession PI 499262. Transcript abundance was estimated from self-aligned RNAseq data measured in transcripts per million (TPM). The expression of the functional resistance gene Sr46 to wheat stem rust (Puccinia graminis f. sp. tritici) is shown.

FIG. 12 is a graphical representation of transcript abundance of NLRs from the de novo assembled transcriptome of Arabidopsis thaliana accession Ler-0 seedlings. Transcript abundance was estimated from self-aligned RNAseq data measured in transcripts per million (TPM). The expression of the functional resistance genes RPP1, RPP5, RPP7, and RPP8 to late blight (Hyaloperonospora arabidopsidis) and WRR4, WRR8, and WRR9 white rust (Albugo candida), are shown.

FIG. 13 is a graphical representation of transcript abundance of NLRs from the de novo assembled transcriptome of Arabidopsis thaliana accession Sf-2 seedlings. Transcript abundance was estimated from self-aligned RNAseq data measured in transcripts per million (TPM). The expression of the functional resistance genes RPP1, RPP5, RPP7, and RPP8 to late blight (Hyaloperonospora arabidopsidis), WRR8 and WRR9 to white rust (Albugo candida), and an allele of RLM3 to grey mould (Botrytis cinerea), dark leaf spot of cabbage (Alternaria brassicicola) and dark spot of crucifers (Alternaria brassicae) is shown.

FIG. 14 is a graphical representation of transcript abundance of NLRs from the de novo assembled transcriptome of Arabidopsis thaliana accession Ws-0 seedlings. Transcript abundance was estimated from self-aligned RNAseq data measured in transcripts per million (TPM). The expression of the functional resistance genes RPP1, RPP5, RPP7, and RPP8 to late blight (Hyaloperonospora arabidopsidis), WRR8 and WRR9 to white rust (Albugo candida), and an allele of RLM3 to grey mould (Botrytis cinerea), dark leaf spot of cabbage (Alternaria brassicicola) and dark spot of crucifers (Alternaria brassicae) is shown.

FIG. 15 is a graphical representation of transcript abundance of NLRs from the de novo assembled transcriptome of Solanum americanum accession 2273. Transcript abundance was estimated from self-aligned RNAseq data measured in transcripts per million (TPM). The expression of the functional resistance gene Rpi-amr1e to late blight (Phytophthora infestans) is shown.

FIG. 16 is a graphical representation of transcript abundance of NLRs from the de novo assembled transcriptome of Solanum lycopersicum cultivar Motelle leaf tissue. Transcript abundance was estimated from self-aligned RNAseq data measured in transcripts per million (TPM). The expression of the functional resistance gene Mi-1.2 to root-knot nematodes (Meloidogyne spp.), the potato aphid (Macrosiphum euphorbiae), and the sweet potato whitefly (Bemisia tabaci) is shown.

FIG. 17 is a graphical representation of transcript abundance of NLRs from the de novo assembled transcriptome of Solanum lycopersicum cultivar Motelle root tissue. Transcript abundance was estimated from self-aligned RNAseq data measured in transcripts per million (TPM). The expression of the functional resistance gene Mi-1.2 to root-knot nematodes (Meloidogyne spp.), the potato aphid (Macrosiphum euphorbiae), and the sweet potato whitefly (Bemisia tabaci) is shown.

FIG. 18 is a graphical representation of transcript abundance of NLRs from the de novo assembled transcriptome of Solanum lycopersicum cultivar VENT Cherry leaf tissue. Transcript abundance was estimated from self-aligned RNAseq data measured in transcripts per million (TPM). The expression of the functional resistance gene Tm-2 to tobamoviruses including Tomato Mosaic Virus (ToMV) and Tobacco Mosaic Virus (TMV) and Mi-1.2 to root-knot nematodes (Meloidogyne spp.), the potato aphid (Macrosiphum euphorbiae), and the sweet potato whitefly (Bemisia tabaci) is shown.

FIG. 19 is a graphical representation of transcript abundance of NLRs from the de novo assembled transcriptome of Solanum lycopersicum cultivar VENT Cherry root tissue. Transcript abundance was estimated from self-aligned RNAseq data measured in transcripts per million (TPM). The expression of the functional resistance gene Tm-2 to tobamoviruses including Tomato Mosaic Virus (ToMV) and Tobacco Mosaic Virus (TMV) and Mi-1.2 to root-knot nematodes (Meloidogyne spp.), the potato aphid (Macrosiphum euphorbiae), and the sweet potato whitefly (Bemisia tabaci) is shown.

### SEQUENCE LISTING

The nucleotide and amino acid sequences listed in the accompanying sequence listing are shown using standard letter abbreviations for nucleotide bases, and three-letter code for amino acids. The nucleotide sequences follow the standard convention of beginning at the 5′ end of the sequence and proceeding forward (i.e., from left to right in each line) to the 3′ end. Only one strand of each nucleotide sequence is shown, but the complementary strand is understood to be included by any reference to the displayed strand. The amino acid sequences follow the standard convention of beginning at the amino terminus of the sequence and proceeding forward (i.e., from left to right in each line) to the carboxy terminus.

SEQ ID NO: 1 sets forth the nucleotide sequence of the coding region of the cDNA of Dk_04_40, an NLR from Aegilops longissima. If desired, a stop codon (e.g. TAA, TAG, or TGA) can be operably linked to the 3′ end of a nucleic acid molecule comprising or consisting of SEQ ID NO: 1. The native stop codon of this cDNA is TAA.

SEQ ID NO: 2 sets forth the amino acid sequence of the NLR protein encoded by Dk_04_40 (SEQ ID NO: 1).

SEQ ID NO: 3 sets forth the nucleotide sequence of the coding region of the cDNA of Dk_01_03, an NLR from Aegilops sharonensis. If desired, a stop codon (e.g. TAA, TAG, or TGA) can be operably linked to the 3′ end of a nucleic acid molecule comprising or consisting of SEQ ID NO: 3. The native stop codon of this cDNA is TGA.

SEQ ID NO: 4 sets forth the amino acid sequence of the NLR protein encoded by Dk_01_03 (SEQ ID NO: 3).

SEQ ID NO: 5 sets forth the nucleotide sequence of the coding region of the cDNA of Dk_01_04, an NLR from Aegilops sharonensis. If desired, a stop codon (e.g. TAA, TAG, or TGA) can be operably linked to the 3′ end of a nucleic acid molecule comprising or consisting of SEQ ID NO: 5. The native stop codon of this cDNA is TGA.

SEQ ID NO: 6 sets forth the amino acid sequence of the NLR protein encoded by Dk_01_04 (SEQ ID NO: 5).

SEQ ID NO: 7 sets forth the nucleotide sequence of the coding region of the cDNA of Dk_01_06, an NLR from Aegilops sharonensis. If desired, a stop codon (e.g. TAA, TAG, or TGA) can be operably linked to the 3′ end of a nucleic acid molecule comprising or consisting of SEQ ID NO: 7. The native stop codon of this cDNA is TAG.

SEQ ID NO: 8 sets forth the amino acid sequence of the NLR protein encoded by Dk_01_06 (SEQ ID NO: 7).

SEQ ID NO: 9 sets forth the nucleotide sequence of the coding region of the cDNA of Dk_01_31, an NLR from Aegilops sharonensis. If desired, a stop codon (e.g. TAA, TAG, or TGA) can be operably linked to the 3′ end of a nucleic acid molecule comprising or consisting of SEQ ID NO: 9. The native stop codon of this cDNA is TAA.

SEQ ID NO: 10 sets forth the amino acid sequence of the NLR protein encoded by Dk_01_31 (SEQ ID NO: 9).

SEQ ID NO: 11 sets forth the nucleotide sequence of the coding region of the cDNA of Dk_01_33, an NLR from Aegilops sharonensis. If desired, a stop codon (e.g. TAA, TAG, or TGA) can be operably linked to the 3′ end of a nucleic acid molecule comprising or consisting of SEQ ID NO: 11. The native stop codon of this cDNA is TGA.

SEQ ID NO: 12 sets forth the amino acid sequence of the NLR protein encoded by Dk_01_33 (SEQ ID NO: 11).

SEQ ID NO: 13 sets forth the nucleotide sequence of the coding region of the cDNA of Dk_01_34, an NLR from Aegilops sharonensis. If desired, a stop codon (e.g. TAA, TAG, or TGA) can be operably linked to the 3′ end of a nucleic acid molecule comprising or consisting of SEQ ID NO: 13. The native stop codon of this cDNA is TGA.

SEQ ID NO: 14 sets forth the amino acid sequence of the NLR protein encoded by Dk_01_34 (SEQ ID NO: 13).

SEQ ID NO: 15 sets forth the nucleotide sequence of the coding region of the cDNA of Dk_01_92, an NLR from Holcus lanatus. If desired, a stop codon (e.g. TAA, TAG, or TGA) can be operably linked to the 3′ end of a nucleic acid molecule comprising or consisting of SEQ ID NO: 15. The native stop codon of this cDNA is TAG.

SEQ ID NO: 16 sets forth the amino acid sequence of the NLR protein encoded by Dk_01_92 (SEQ ID NO: 15).

SEQ ID NO: 17 sets forth the nucleotide sequence of the coding region of the cDNA of Dk_02_27, an NLR from Koeleria macrantha. If desired, a stop codon (e.g. TAA, TAG, or TGA) can be operably linked to the 3′ end of a nucleic acid molecule comprising or consisting of SEQ ID NO: 17. The native stop codon of this cDNA is TAA.

SEQ ID NO: 18 sets forth the amino acid sequence of the NLR protein encoded by Dk_02_27 (SEQ ID NO: 17).

SEQ ID NO: 19 sets forth the nucleotide sequence of the coding region of the cDNA of Dk_02_28, an NLR from Koeleria macrantha. If desired, a stop codon (e.g. TAA, TAG, or TGA) can be operably linked to the 3′ end of a nucleic acid molecule comprising or consisting of SEQ ID NO: 19. The native stop codon of this cDNA is TAG.

SEQ ID NO: 20 sets forth the amino acid sequence of the NLR protein encoded by Dk_02_28 (SEQ ID NO: 19).

SEQ ID NO: 21 sets forth the nucleotide sequence of the coding region of the cDNA of Dk_02_49, an NLR from Koeleria macrantha. If desired, a stop codon (e.g. TAA, TAG, or TGA) can be operably linked to the 3′ end of a nucleic acid molecule comprising or consisting of SEQ ID NO: 21. The native stop codon of this cDNA is TAA.

SEQ ID NO: 22 sets forth the amino acid sequence of the NLR protein encoded by Dk_02_49 (SEQ ID NO: 21).

SEQ ID NO: 23 sets forth the nucleotide sequence of the coding region of the cDNA of Dk_03_76, an NLR from Koeleria macrantha. If desired, a stop codon (e.g. TAA, TAG, or TGA) can be operably linked to the 3′ end of a nucleic acid molecule comprising or consisting of SEQ ID NO: 23. The native stop codon of this cDNA is TGA.

SEQ ID NO: 24 sets forth the amino acid sequence of the NLR protein encoded by Dk_03_76 (SEQ ID NO: 23).

SEQ ID NO: 25 sets forth the nucleotide sequence of the coding region of the cDNA of Dk_01_19, an NLR from Aegilops sharonensis. If desired, a stop codon (e.g. TAA, TAG, or TGA) can be operably linked to the 3′ end of a nucleic acid molecule comprising or consisting of SEQ ID NO: 25. The native stop codon of this cDNA is TGA.

SEQ ID NO: 26 sets forth the amino acid sequence of the NLR protein encoded by Dk_01_19 (SEQ ID NO: 25).

SEQ ID NO: 27 sets forth the nucleotide sequence of the Gateway adapter attB1.

SEQ ID NO: 28 sets forth the nucleotide sequence of the Gateway adapter attB2.

SEQ ID NO: 29 sets forth the nucleotide sequence of the coding region of the cDNA of Dk_01_35, an NLR from Aegilops sharonensis. If desired, a stop codon (e.g. TAA, TAG, or TGA) can be operably linked to the 3′ end of a nucleic acid molecule comprising or consisting of this NLR sequence. The native stop codon of this cDNA is TGA.

SEQ ID NO: 30 sets forth the amino acid sequence of the NLR protein encoded by SEQ ID NO: 29.

SEQ ID NO: 31 sets forth the nucleotide sequence of the coding region of the cDNA of Dk_01_55, an NLR from Aegilops sharonensis. If desired, a stop codon (e.g. TAA, TAG, or TGA) can be operably linked to the 3′ end of a nucleic acid molecule comprising or consisting of this NLR sequence. The native stop codon of this cDNA is TGA.

SEQ ID NO: 32 sets forth the amino acid sequence of the NLR protein encoded by SEQ ID NO: 31.

SEQ ID NO: 33 sets forth the nucleotide sequence of the coding region of the cDNA of Dk_01_57, an NLR from Aegilops sharonensis. If desired, a stop codon (e.g. TAA, TAG, or TGA) can be operably linked to the 3′ end of a nucleic acid molecule comprising or consisting of this NLR sequence. The native stop codon of this cDNA is TGA.

SEQ ID NO: 34 sets forth the amino acid sequence of the NLR protein encoded by SEQ ID NO: 33.

SEQ ID NO: 35 sets forth the nucleotide sequence of the coding region of the cDNA of Dk_01_59, an NLR from Aegilops sharonensis. If desired, a stop codon (e.g. TAA, TAG, or TGA) can be operably linked to the 3′ end of a nucleic acid molecule comprising or consisting of this NLR sequence. The native stop codon of this cDNA is TAA.

SEQ ID NO: 36 sets forth the amino acid sequence of the NLR protein encoded by SEQ ID NO: 35.

SEQ ID NO: 37 sets forth the nucleotide sequence of the coding region of the cDNA of Dk_01_60, an NLR from Aegilops sharonensis. If desired, a stop codon (e.g. TAA, TAG, or TGA) can be operably linked to the 3′ end of a nucleic acid molecule comprising or consisting of this NLR sequence. The native stop codon of this cDNA is TAG.

SEQ ID NO: 38 sets forth the amino acid sequence of the NLR protein encoded by SEQ ID NO: 37)

SEQ ID NO: 39 sets forth the nucleotide sequence of the coding region of the cDNA of Dk_01_61, an NLR from Cynosurus cristatus. If desired, a stop codon (e.g. TAA, TAG, or TGA) can be operably linked to the 3′ end of a nucleic acid molecule comprising or consisting of this NLR sequence. The native stop codon of this cDNA is TAA.

SEQ ID NO: 40 sets forth the amino acid sequence of the NLR protein encoded by SEQ ID NO: 39.

SEQ ID NO: 41 sets forth the nucleotide sequence of the coding region of the cDNA of Dk_01_62, an NLR from Cynosurus cristatus. If desired, a stop codon (e.g. TAA, TAG, or TGA) can be operably linked to the 3′ end of a nucleic acid molecule comprising or consisting of this NLR sequence. The native stop codon of this cDNA is TGA.

SEQ ID NO: 42 sets forth the amino acid sequence of the NLR protein encoded by SEQ ID NO: 41.

SEQ ID NO: 43 sets forth the nucleotide sequence of the coding region of the cDNA of Dk_01_64, an NLR from Cynosurus cristatus. If desired, a stop codon (e.g. TAA, TAG, or TGA) can be operably linked to the 3′ end of a nucleic acid molecule comprising or consisting of this NLR sequence. The native stop codon of this cDNA is TGA.

SEQ ID NO: 44 sets forth the amino acid sequence of the NLR protein encoded by SEQ ID NO: 43.

SEQ ID NO: 45 sets forth the nucleotide sequence of the coding region of the cDNA of Dk_01_68, an NLR from Cynosurus cristatus. If desired, a stop codon (e.g. TAA, TAG, or TGA) can be operably linked to the 3′ end of a nucleic acid molecule comprising or consisting of this NLR sequence. The native stop codon of this cDNA is TGA.

SEQ ID NO: 46 sets forth the amino acid sequence of the NLR protein encoded by SEQ ID NO: 45.

SEQ ID NO: 47 sets forth the nucleotide sequence of the coding region of the cDNA of Dk_02_02, an NLR from Koeleria macrantha. If desired, a stop codon (e.g. TAA, TAG, or TGA) can be operably linked to the 3′ end of a nucleic acid molecule comprising or consisting of this NLR sequence. The native stop codon of this cDNA is TAG.

SEQ ID NO: 48 sets forth the amino acid sequence of the NLR protein encoded by SEQ ID NO: 47.

SEQ ID NO: 49 sets forth the nucleotide sequence of the coding region of the cDNA of Dk_02_03, an NLR from Koeleria macrantha. If desired, a stop codon (e.g. TAA, TAG, or TGA) can be operably linked to the 3′ end of a nucleic acid molecule comprising or consisting of this NLR sequence. The native stop codon of this cDNA is TAA.

SEQ ID NO: 50 sets forth the amino acid sequence of the NLR protein encoded by SEQ ID NO: 49.

SEQ ID NO: 51 sets forth the nucleotide sequence of the coding region of the cDNA of Dk_02_06, an NLR from Koeleria macrantha. If desired, a stop codon (e.g. TAA, TAG, or TGA) can be operably linked to the 3′ end of a nucleic acid molecule comprising or consisting of this NLR sequence. The native stop codon of this cDNA is TAG.

SEQ ID NO: 52 sets forth the amino acid sequence of the NLR protein encoded by SEQ ID NO: 51.

SEQ ID NO: 53 sets forth the nucleotide sequence of the coding region of the cDNA of Dk_02_07, an NLR from Koeleria macrantha. If desired, a stop codon (e.g. TAA, TAG, or TGA) can be operably linked to the 3′ end of a nucleic acid molecule comprising or consisting of this NLR sequence. The native stop codon of this cDNA is TAA.

SEQ ID NO: 54 sets forth the amino acid sequence of the NLR protein encoded by SEQ ID NO: 53.

SEQ ID NO: 55 sets forth the nucleotide sequence of the coding region of the cDNA of Dk_02_08, an NLR from Koeleria macrantha. If desired, a stop codon (e.g. TAA, TAG, or TGA) can be operably linked to the 3′ end of a nucleic acid molecule comprising or consisting of this NLR sequence. The native stop codon of this cDNA is TAG.

SEQ ID NO: 56 sets forth the amino acid sequence of the NLR protein encoded by SEQ ID NO: 55.

SEQ ID NO: 57 sets forth the nucleotide sequence of the coding region of the cDNA of Dk_02_11, an NLR from Koeleria macrantha. If desired, a stop codon (e.g. TAA, TAG, or TGA) can be operably linked to the 3′ end of a nucleic acid molecule comprising or consisting of this NLR sequence. The native stop codon of this cDNA is TAA.

SEQ ID NO: 58 sets forth the amino acid sequence of the NLR protein encoded by SEQ ID NO: 57.

SEQ ID NO: 59 sets forth the nucleotide sequence of the coding region of the cDNA of Dk_02_13, an NLR from Koeleria macrantha. If desired, a stop codon (e.g. TAA, TAG, or TGA) can be operably linked to the 3′ end of a nucleic acid molecule comprising or consisting of this NLR sequence. The native stop codon of this cDNA is TGA.

SEQ ID NO: 60 sets forth the amino acid sequence of the NLR protein encoded by SEQ ID NO: 59.

SEQ ID NO: 61 sets forth the nucleotide sequence of the coding region of the cDNA of Dk_02_14, an NLR from Koeleria macrantha. If desired, a stop codon (e.g. TAA, TAG, or TGA) can be operably linked to the 3′ end of a nucleic acid molecule comprising or consisting of this NLR sequence. The native stop codon of this cDNA is TGA.

SEQ ID NO: 62 sets forth the amino acid sequence of the NLR protein encoded by SEQ ID NO: 61.

SEQ ID NO: 63 sets forth the nucleotide sequence of the coding region of the cDNA of Dk_02_19, an NLR from Koeleria macrantha. If desired, a stop codon (e.g. TAA, TAG, or TGA) can be operably linked to the 3′ end of a nucleic acid molecule comprising or consisting of this NLR sequence. The native stop codon of this cDNA is TAG.

SEQ ID NO: 64 sets forth the amino acid sequence of the NLR protein encoded by SEQ ID NO: 63.

SEQ ID NO: 65 sets forth the nucleotide sequence of the coding region of the cDNA of Dk_02_20, an NLR from Koeleria macrantha. If desired, a stop codon (e.g. TAA, TAG, or TGA) can be operably linked to the 3′ end of a nucleic acid molecule comprising or consisting of this NLR sequence. The native stop codon of this cDNA is TGA.

SEQ ID NO: 66 sets forth the amino acid sequence of the NLR protein encoded by SEQ ID NO: 65.

SEQ ID NO: 67 sets forth the nucleotide sequence of the coding region of the cDNA of Dk_02_25, an NLR from Koeleria macrantha. If desired, a stop codon (e.g. TAA, TAG, or TGA) can be operably linked to the 3′ end of a nucleic acid molecule comprising or consisting of this NLR sequence. The native stop codon of this cDNA is TGA.

SEQ ID NO: 68 sets forth the amino acid sequence of the NLR protein encoded by SEQ ID NO: 67.

SEQ ID NO: 69 sets forth the nucleotide sequence of the coding region of the cDNA of Dk_02_34, an NLR from Koeleria macrantha. If desired, a stop codon (e.g. TAA, TAG, or TGA) can be operably linked to the 3′ end of a nucleic acid molecule comprising or consisting of this NLR sequence. The native stop codon of this cDNA is TAG.

SEQ ID NO: 70 sets forth the amino acid sequence of the NLR protein encoded by SEQ ID NO: 69.

SEQ ID NO: 71 sets forth the nucleotide sequence of the coding region of the cDNA of Dk_02_35, an NLR from Koeleria macrantha. If desired, a stop codon (e.g. TAA, TAG, or TGA) can be operably linked to the 3′ end of a nucleic acid molecule comprising or consisting of this NLR sequence. The native stop codon of this cDNA is TGA.

SEQ ID NO: 72 sets forth the amino acid sequence of the NLR protein encoded by SEQ ID NO: 71.

SEQ ID NO: 73 sets forth the nucleotide sequence of the coding region of the cDNA of Dk_02_36, an NLR from Koeleria macrantha. If desired, a stop codon (e.g. TAA, TAG, or TGA) can be operably linked to the 3′ end of a nucleic acid molecule comprising or consisting of this NLR sequence. The native stop codon of this cDNA is TAA.

SEQ ID NO: 74 sets forth the amino acid sequence of the NLR protein encoded by SEQ ID NO: 73.

SEQ ID NO: 75 sets forth the nucleotide sequence of the coding region of the cDNA of Dk_02_38, an NLR from Koeleria macrantha. If desired, a stop codon (e.g. TAA, TAG, or TGA) can be operably linked to the 3′ end of a nucleic acid molecule comprising or consisting of this NLR sequence. The native stop codon of this cDNA is TGA,

SEQ ID NO: 76 sets forth the amino acid sequence of the NLR protein encoded by SEQ ID NO: 75.

SEQ ID NO: 77 sets forth the nucleotide sequence of the coding region of the cDNA of Dk_02_39, an NLR from Koeleria macrantha. If desired, a stop codon (e.g. TAA, TAG, or TGA) can be operably linked to the 3′ end of a nucleic acid molecule comprising or consisting of this NLR sequence. The native stop codon of this cDNA is TAA.

SEQ ID NO: 78 sets forth the amino acid sequence of the NLR protein encoded by SEQ ID NO: 77.

SEQ ID NO: 79 sets forth the nucleotide sequence of the coding region of the cDNA of Dk_02_42, an NLR from Koeleria macrantha. If desired, a stop codon (e.g. TAA, TAG, or TGA) can be operably linked to the 3′ end of a nucleic acid molecule comprising or consisting of this NLR sequence. The native stop codon of this cDNA is TGA.

SEQ ID NO: 80 sets forth the amino acid sequence of the NLR protein encoded by SEQ ID NO: 79.

SEQ ID NO: 81 sets forth the nucleotide sequence of the coding region of the cDNA of Dk_02_44, an NLR from Koeleria macrantha. If desired, a stop codon (e.g. TAA, TAG, or TGA) can be operably linked to the 3′ end of a nucleic acid molecule comprising or consisting of this NLR sequence. The native stop codon of this cDNA is TAA.

SEQ ID NO: 82 sets forth the amino acid sequence of the NLR protein encoded by SEQ ID NO: 81.

SEQ ID NO: 83 sets forth the nucleotide sequence of the coding region of the cDNA of Dk_02_46, an NLR from Koeleria macrantha. If desired, a stop codon (e.g. TAA, TAG, or TGA) can be operably linked to the 3′ end of a nucleic acid molecule comprising or consisting of this NLR sequence. The native stop codon of this cDNA is TGA.

SEQ ID NO: 84 sets forth the amino acid sequence of the NLR protein encoded by SEQ ID NO: 83.

SEQ ID NO: 85 sets forth the nucleotide sequence of the coding region of the cDNA of Dk_03_13, an NLR from Cynosurus cristatus. If desired, a stop codon (e.g. TAA, TAG, or TGA) can be operably linked to the 3′ end of a nucleic acid molecule comprising or consisting of this NLR sequence. The native stop codon of this cDNA is TGA.

SEQ ID NO: 86 sets forth the amino acid sequence of the NLR protein encoded by SEQ ID NO: 85.

SEQ ID NO: 87 sets forth the nucleotide sequence of the coding region of the cDNA of Dk_03_16, an NLR from Cynosurus cristatus. If desired, a stop codon (e.g. TAA, TAG, or TGA) can be operably linked to the 3′ end of a nucleic acid molecule comprising or consisting of this NLR sequence. The native stop codon of this cDNA is TAA.

SEQ ID NO: 88 sets forth the amino acid sequence of the NLR protein encoded by SEQ ID NO: 87.

SEQ ID NO: 89 sets forth the nucleotide sequence of the coding region of the cDNA of Dk_03_19, an NLR from Cynosurus cristatus. If desired, a stop codon (e.g. TAA, TAG, or TGA) can be operably linked to the 3′ end of a nucleic acid molecule comprising or consisting of this NLR sequence. The native stop codon of this cDNA is TAG.

SEQ ID NO: 90 sets forth the amino acid sequence of the NLR protein encoded by SEQ ID NO: 89.

SEQ ID NO: 91 sets forth the nucleotide sequence of the coding region of the cDNA of Dk_03_48, an NLR from Holcus lanatus. If desired, a stop codon (e.g. TAA, TAG, or TGA) can be operably linked to the 3′ end of a nucleic acid molecule comprising or consisting of this NLR sequence. The native stop codon of this cDNA is TAA.

SEQ ID NO: 92 sets forth the amino acid sequence of the NLR protein encoded by SEQ ID NO: 91.

SEQ ID NO: 93 sets forth the nucleotide sequence of the coding region of the cDNA of Dk_03_58, an NLR from Koeleria macrantha. If desired, a stop codon (e.g. TAA, TAG, or TGA) can be operably linked to the 3′ end of a nucleic acid molecule comprising or consisting of this NLR sequence. The native stop codon of this cDNA is TAA.

SEQ ID NO: 94 sets forth the amino acid sequence of the NLR protein encoded by SEQ ID NO: 93.

SEQ ID NO: 95 sets forth the nucleotide sequence of the coding region of the cDNA of Dk_03_60, an NLR from Koeleria macrantha. If desired, a stop codon (e.g. TAA, TAG, or TGA) can be operably linked to the 3′ end of a nucleic acid molecule comprising or consisting of this NLR sequence. The native stop codon of this cDNA is TGA.

SEQ ID NO: 96 sets forth the amino acid sequence of the NLR protein encoded by SEQ ID NO: 95.

SEQ ID NO: 97 sets forth the nucleotide sequence of the coding region of the cDNA of Dk_04_34, an NLR from Hordeum vulgare. If desired, a stop codon (e.g. TAA, TAG, or TGA) can be operably linked to the 3′ end of a nucleic acid molecule comprising or consisting of this NLR sequence. The native stop codon of this cDNA is TAG.

SEQ ID NO: 98 sets forth the amino acid sequence of the NLR protein encoded by SEQ ID NO: 97.

SEQ ID NO: 99 sets forth the nucleotide sequence of the coding region of the cDNA of Dk_04_44, an NLR from Aegilops bicornis. If desired, a stop codon (e.g. TAA, TAG, or TGA) can be operably linked to the 3′ end of a nucleic acid molecule comprising or consisting of this NLR sequence. The native stop codon of this cDNA is TGA.

SEQ ID NO: 100 sets forth the amino acid sequence of the NLR protein encoded by SEQ ID NO: 99.

SEQ ID NO: 101 sets forth the nucleotide sequence of the coding region of the cDNA of Dk_04_85, an NLR from Aegilops bicornis. If desired, a stop codon (e.g. TAA, TAG, or TGA) can be operably linked to the 3′ end of a nucleic acid molecule comprising or consisting of this NLR sequence. The native stop codon of this cDNA is TAA.

SEQ ID NO: 102 sets forth the amino acid sequence of the NLR protein encoded by SEQ ID NO: 101.

SEQ ID NO: 103 sets forth the nucleotide sequence of the coding region of the cDNA of Dk_04_88, an NLR from Aegilops bicornis. If desired, a stop codon (e.g. TAA, TAG, or TGA) can be operably linked to the 3′ end of a nucleic acid molecule comprising or consisting of this NLR sequence. The native stop codon of this cDNA is TGA.

SEQ ID NO: 104 sets forth the amino acid sequence of the NLR protein encoded by SEQ ID NO: 103.

SEQ ID NO: 105 sets forth the nucleotide sequence of the coding region of the cDNA of Dk_04_92, an NLR from Aegilops bicornis. If desired, a stop codon (e.g. TAA, TAG, or TGA) can be operably linked to the 3′ end of a nucleic acid molecule comprising or consisting of this NLR sequence. The native stop codon of this cDNA is TGA.

SEQ ID NO: 106 sets forth the amino acid sequence of the NLR protein encoded by SEQ ID NO: 105.

SEQ ID NO: 107 sets forth the nucleotide sequence of the coding region of the cDNA of Dk_04_95, an NLR from Aegilops bicornis. If desired, a stop codon (e.g. TAA, TAG, or TGA) can be operably linked to the 3′ end of a nucleic acid molecule comprising or consisting of this NLR sequence. The native stop codon of this cDNA is TAA.

SEQ ID NO: 108 sets forth the amino acid sequence of the NLR protein encoded by SEQ ID NO: 107.

SEQ ID NO: 109 sets forth the nucleotide sequence of the coding region of the cDNA of Dk_04_96, an NLR from Aegilops bicornis. If desired, a stop codon (e.g. TAA, TAG, or TGA) can be operably linked to the 3′ end of a nucleic acid molecule comprising or consisting of this NLR sequence. The native stop codon of this cDNA is TGA.

SEQ ID NO: 110 sets forth the amino acid sequence of the NLR protein encoded by SEQ ID NO: 109.

SEQ ID NO: 111 sets forth the nucleotide sequence of the coding region of the cDNA of Dk_05_11, an NLR from Aegilops longissima. If desired, a stop codon (e.g. TAA, TAG, or TGA) can be operably linked to the 3′ end of a nucleic acid molecule comprising or consisting of this NLR sequence. The native stop codon of this cDNA is TGA.

SEQ ID NO: 112 sets forth the amino acid sequence of the NLR protein encoded by SEQ ID NO: 111.

SEQ ID NO: 113 sets forth the nucleotide sequence of the coding region of the cDNA of Dk_05_14, an NLR from Aegilops longissima. If desired, a stop codon (e.g. TAA, TAG, or TGA) can be operably linked to the 3′ end of a nucleic acid molecule comprising or consisting of this NLR sequence. The native stop codon of this cDNA is TAA.

SEQ ID NO: 114 sets forth the amino acid sequence of the NLR protein encoded by SEQ ID NO: 113.

SEQ ID NO: 115 sets forth the nucleotide sequence of the coding region of the cDNA of Dk_05_15, an NLR from Aegilops longissima. If desired, a stop codon (e.g. TAA, TAG, or TGA) can be operably linked to the 3′ end of a nucleic acid molecule comprising or consisting of this NLR sequence. The native stop codon of this cDNA is TGA.

SEQ ID NO: 116 sets forth the amino acid sequence of the NLR protein encoded by SEQ ID NO: 115.

SEQ ID NO: 117 sets forth the nucleotide sequence of the coding region of the cDNA of Dk_05_16, an NLR from Aegilops longissima. If desired, a stop codon (e.g. TAA, TAG, or TGA) can be operably linked to the 3′ end of a nucleic acid molecule comprising or consisting of this NLR sequence. The native stop codon of this cDNA is TGA.

SEQ ID NO: 118 sets forth the amino acid sequence of the NLR protein encoded by SEQ ID NO: 117.

SEQ ID NO: 119 sets forth the nucleotide sequence of the coding region of the cDNA of Dk_05_24, an NLR from Aegilops longissima. If desired, a stop codon (e.g. TAA, TAG, or TGA) can be operably linked to the 3′ end of a nucleic acid molecule comprising or consisting of this NLR sequence. The native stop codon of this cDNA is TAA.

SEQ ID NO: 120 sets forth the amino acid sequence of the NLR protein encoded by SEQ ID NO: 119.

SEQ ID NO: 121 sets forth the nucleotide sequence of the coding region of the cDNA of Dk_05_29, an NLR from Aegilops longissima. If desired, a stop codon (e.g. TAA, TAG, or TGA) can be operably linked to the 3′ end of a nucleic acid molecule comprising or consisting of this NLR sequence. The native stop codon of this cDNA is TAA.

SEQ ID NO: 122 sets forth the amino acid sequence of the NLR protein encoded by SEQ ID NO: 121.

SEQ ID NO: 123 sets forth the nucleotide sequence of the coding region of the cDNA of Dk_05_30, an NLR from Aegilops longissima. If desired, a stop codon (e.g. TAA, TAG, or TGA) can be operably linked to the 3′ end of a nucleic acid molecule comprising or consisting of this NLR sequence. The native stop codon of this cDNA is TGA.

SEQ ID NO: 124 sets forth the amino acid sequence of the NLR protein encoded by SEQ ID NO: 123.

SEQ ID NO: 125 sets forth the nucleotide sequence of the coding region of the cDNA of Dk_05_33, an NLR from Aegilops longissima. If desired, a stop codon (e.g. TAA, TAG, or TGA) can be operably linked to the 3′ end of a nucleic acid molecule comprising or consisting of this NLR sequence. The native stop codon of this cDNA is TAG.

SEQ ID NO: 126 sets forth the amino acid sequence of the NLR protein encoded by SEQ ID NO: 125.

SEQ ID NO: 127 sets forth the nucleotide sequence of the coding region of the cDNA of Dk_05_34, an NLR from Aegilops longissima. If desired, a stop codon (e.g. TAA, TAG, or TGA) can be operably linked to the 3′ end of a nucleic acid molecule comprising or consisting of this NLR sequence. The native stop codon of this cDNA is TAG.

SEQ ID NO: 128 sets forth the amino acid sequence of the NLR protein encoded by SEQ ID NO: 127.

SEQ ID NO: 129 sets forth the nucleotide sequence of the coding region of the cDNA of Dk_05_35, an NLR from Aegilops longissima. If desired, a stop codon (e.g. TAA, TAG, or TGA) can be operably linked to the 3′ end of a nucleic acid molecule comprising or consisting of this NLR sequence. The native stop codon of this cDNA is TAA.

SEQ ID NO: 130 sets forth the amino acid sequence of the NLR protein encoded by SEQ ID NO: 129.

SEQ ID NO: 131 sets forth the nucleotide sequence of the coding region of the cDNA of Dk_05_38, an NLR from Aegilops longissima. If desired, a stop codon (e.g. TAA, TAG, or TGA) can be operably linked to the 3′ end of a nucleic acid molecule comprising or consisting of this NLR sequence. The native stop codon of this cDNA is TGA.

SEQ ID NO: 132 sets forth the amino acid sequence of the NLR protein encoded by SEQ ID NO: 131.

SEQ ID NO: 133 sets forth the nucleotide sequence of the coding region of the cDNA of Dk_05_42, an NLR from Aegilops longissima. If desired, a stop codon (e.g. TAA, TAG, or TGA) can be operably linked to the 3′ end of a nucleic acid molecule comprising or consisting of this NLR sequence. The native stop codon of this cDNA is TGA.

SEQ ID NO: 134 sets forth the amino acid sequence of the NLR protein encoded by SEQ ID NO: 133.

SEQ ID NO: 135 sets forth the nucleotide sequence of the coding region of the cDNA of Dk_05_44, an NLR from Aegilops longissima. If desired, a stop codon (e.g. TAA, TAG, or TGA) can be operably linked to the 3′ end of a nucleic acid molecule comprising or consisting of this NLR sequence. The native stop codon of this cDNA is TAA.

SEQ ID NO: 136 sets forth the amino acid sequence of the NLR protein encoded by SEQ ID NO: 135.

SEQ ID NO: 137 sets forth the nucleotide sequence of the coding region of the cDNA of Dk_05_47, an NLR from Aegilops longissima. If desired, a stop codon (e.g. TAA, TAG, or TGA) can be operably linked to the 3′ end of a nucleic acid molecule comprising or consisting of this NLR sequence. The native stop codon of this cDNA is TGA.

SEQ ID NO: 138 sets forth the amino acid sequence of the NLR protein encoded by SEQ ID NO: 137.

SEQ ID NO: 139 sets forth the nucleotide sequence of the coding region of the cDNA of Dk_05_53, an NLR from Aegilops longissima. If desired, a stop codon (e.g. TAA, TAG, or TGA) can be operably linked to the 3′ end of a nucleic acid molecule comprising or consisting of this NLR sequence. The native stop codon of this cDNA is TAA.

SEQ ID NO: 140 sets forth the amino acid sequence of the NLR protein encoded by SEQ ID NO: 139.

SEQ ID NO: 141 sets forth the nucleotide sequence of the coding region of the cDNA of Dk_05_56, an NLR from Aegilops longissima. If desired, a stop codon (e.g. TAA, TAG, or TGA) can be operably linked to the 3′ end of a nucleic acid molecule comprising or consisting of this NLR sequence. The native stop codon of this cDNA is TGA.

SEQ ID NO: 142 sets forth the amino acid sequence of the NLR protein encoded by SEQ ID NO: 141.

SEQ ID NO: 143 sets forth the nucleotide sequence of the coding region of the cDNA of Dk_06_01, an NLR from Brachypodium distachyon. If desired, a stop codon (e.g. TAA, TAG, or TGA) can be operably linked to the 3′ end of a nucleic acid molecule comprising or consisting of this NLR sequence. The native stop codon of this cDNA is TAA.

SEQ ID NO: 144 sets forth the amino acid sequence of the NLR protein encoded by SEQ ID NO: 143.

SEQ ID NO: 145 sets forth the nucleotide sequence of the coding region of the cDNA of Dk_06_03, an NLR from Aegilops longissima. If desired, a stop codon (e.g. TAA, TAG, or TGA) can be operably linked to the 3′ end of a nucleic acid molecule comprising or consisting of this NLR sequence. The native stop codon of this cDNA is TAA.

SEQ ID NO: 146 sets forth the amino acid sequence of the NLR protein encoded by SEQ ID NO: 145.

SEQ ID NO: 147 sets forth the nucleotide sequence of the coding region of the cDNA of Dk_06_04, an NLR from Aegilops longissima. If desired, a stop codon (e.g. TAA, TAG, or TGA) can be operably linked to the 3′ end of a nucleic acid molecule comprising or consisting of this NLR sequence. The native stop codon of this cDNA is TAA.

SEQ ID NO: 148 sets forth the amino acid sequence of the NLR protein encoded by SEQ ID NO: 147.

SEQ ID NO: 149 sets forth the nucleotide sequence of the coding region of the cDNA of Dk_06_05, an NLR from Aegilops longissima. If desired, a stop codon (e.g. TAA, TAG, or TGA) can be operably linked to the 3′ end of a nucleic acid molecule comprising or consisting of this NLR sequence. The native stop codon of this cDNA is TAA.

SEQ ID NO: 150 sets forth the amino acid sequence of the NLR protein encoded by SEQ ID NO: 149.

SEQ ID NO: 151 sets forth the nucleotide sequence of the coding region of the cDNA of Dk_06_06, an NLR from Aegilops longissima. If desired, a stop codon (e.g. TAA, TAG, or TGA) can be operably linked to the 3′ end of a nucleic acid molecule comprising or consisting of this NLR sequence. The native stop codon of this cDNA is TGA.

SEQ ID NO: 152 sets forth the amino acid sequence of the NLR protein encoded by SEQ ID NO: 151.

SEQ ID NO: 153 sets forth the nucleotide sequence of the coding region of the cDNA of Dk_06_52, an NLR from Aegilops searsii. If desired, a stop codon (e.g. TAA, TAG, or TGA) can be operably linked to the 3′ end of a nucleic acid molecule comprising or consisting of this NLR sequence. The native stop codon of this cDNA is TGA.

SEQ ID NO: 154 sets forth the amino acid sequence of the NLR protein encoded by SEQ ID NO: 153.

SEQ ID NO: 155 sets forth the nucleotide sequence of the coding region of the cDNA of Dk_06_53, an NLR from Aegilops searsii. If desired, a stop codon (e.g. TAA, TAG, or TGA) can be operably linked to the 3′ end of a nucleic acid molecule comprising or consisting of this NLR sequence. The native stop codon of this cDNA is TAG.

SEQ ID NO: 156 sets forth the amino acid sequence of the NLR protein encoded by SEQ ID NO: 155.

SEQ ID NO: 157 sets forth the nucleotide sequence of the coding region of the cDNA of Dk_01_21, an NLR from Aegilops sharonensis. If desired, a stop codon (e.g. TAA, TAG, or TGA) can be operably linked to the 3′ end of a nucleic acid molecule comprising or consisting of this NLR sequence. The native stop codon of this cDNA is TAA.

SEQ ID NO: 158 sets forth the amino acid sequence of the NLR protein encoded by SEQ ID NO: 157.

SEQ ID NO: 159 sets forth the nucleotide sequence of the coding region of the cDNA of Dk_01_48, an NLR from Aegilops sharonensis. If desired, a stop codon (e.g. TAA, TAG, or TGA) can be operably linked to the 3′ end of a nucleic acid molecule comprising or consisting of this NLR sequence. The native stop codon of this cDNA is TAG.

SEQ ID NO: 160 sets forth the amino acid sequence of the NLR protein encoded by SEQ ID NO: 159.

SEQ ID NO: 161 sets forth the nucleotide sequence of the coding region of the cDNA of Dk_03_15, an NLR from Cynosurus cristatus. If desired, a stop codon (e.g. TAA, TAG, or TGA) can be operably linked to the 3′ end of a nucleic acid molecule comprising or consisting of this NLR sequence. The native stop codon of this cDNA is TGA.

SEQ ID NO: 162 sets forth the amino acid sequence of the NLR protein encoded by SEQ ID NO: 161.

SEQ ID NO: 163 sets forth the nucleotide sequence of the coding region of the cDNA of Dk_03_49, an NLR from Holcus lanatus. If desired, a stop codon (e.g. TAA, TAG, or TGA) can be operably linked to the 3′ end of a nucleic acid molecule comprising or consisting of this NLR sequence. The native stop codon of this cDNA is TGA.

SEQ ID NO: 164 sets forth the amino acid sequence of the NLR protein encoded by SEQ ID NO: 163.

SEQ ID NO: 165 sets forth the nucleotide sequence of the coding region of the cDNA of Dk_03_68, an NLR from Aegilops sharonensis. If desired, a stop codon (e.g. TAA, TAG, or TGA) can be operably linked to the 3′ end of a nucleic acid molecule comprising or consisting of this NLR sequence. The native stop codon of this cDNA is TAG.

SEQ ID NO: 166 sets forth the amino acid sequence of the NLR protein encoded by SEQ ID NO: 165.

SEQ ID NO: 167 sets forth the nucleotide sequence of the coding region of the cDNA of Dk_04_67, an NLR from Aegilops bicornis. If desired, a stop codon (e.g. TAA, TAG, or TGA) can be operably linked to the 3′ end of a nucleic acid molecule comprising or consisting of this NLR sequence. The native stop codon of this cDNA is TGA.

SEQ ID NO: 168 sets forth the amino acid sequence of the NLR protein encoded by SEQ ID NO: 167.

SEQ ID NO: 169 sets forth the nucleotide sequence of the coding region of the cDNA of Dk_04_71, an NLR from Aegilops bicornis. If desired, a stop codon (e.g. TAA, TAG, or TGA) can be operably linked to the 3′ end of a nucleic acid molecule comprising or consisting of this NLR sequence. The native stop codon of this cDNA is TAG.

SEQ ID NO: 170 sets forth the amino acid sequence of the NLR protein encoded by SEQ ID NO: 169.

SEQ ID NO: 171 sets forth the nucleotide sequence of the coding region of the cDNA of Dk_04_91, an NLR from Aegilops bicornis. If desired, a stop codon (e.g. TAA, TAG, or TGA) can be operably linked to the 3′ end of a nucleic acid molecule comprising or consisting of this NLR sequence. The native stop codon of this cDNA is TAG.

SEQ ID NO: 172 sets forth the amino acid sequence of the NLR protein encoded by SEQ ID NO: 171.

SEQ ID NO: 173 sets forth the nucleotide sequence of the coding region of the cDNA of Dk_05_75, an NLR from Aegilops bicornis. If desired, a stop codon (e.g. TAA, TAG, or TGA) can be operably linked to the 3′ end of a nucleic acid molecule comprising or consisting of this NLR sequence. The native stop codon of this cDNA is TGA.

SEQ ID NO: 174 sets forth the amino acid sequence of the NLR protein encoded by SEQ ID NO: 173.

SEQ ID NO: 175 sets forth the nucleotide sequence of the coding region of the cDNA of Dk_05_92, an NLR from Aegilops bicornis. If desired, a stop codon (e.g. TAA, TAG, or TGA) can be operably linked to the 3′ end of a nucleic acid molecule comprising or consisting of this NLR sequence. The native stop codon of this cDNA is TAG.

SEQ ID NO: 176 sets forth the amino acid sequence of the NLR protein encoded by SEQ ID NO: 175.

SEQ ID NO: 177 sets forth the nucleotide sequence of the coding region of the cDNA of Dk_06_02, an NLR from Aegilops longissima. If desired, a stop codon (e.g. TAA, TAG, or TGA) can be operably linked to the 3′ end of a nucleic acid molecule comprising or consisting of this NLR sequence. The native stop codon of this cDNA is TAG.

SEQ ID NO: 178 sets forth the amino acid sequence of the NLR protein encoded by SEQ ID NO: 177.

SEQ ID NO: 179 sets forth the nucleotide sequence of the coding region of the cDNA of Dk_06_10, an NLR from Aegilops bicornis. If desired, a stop codon (e.g. TAA, TAG, or TGA) can be operably linked to the 3′ end of a nucleic acid molecule comprising or consisting of this NLR sequence. The native stop codon of this cDNA is TAG.

SEQ ID NO: 180 sets forth the amino acid sequence of the NLR protein encoded by SEQ ID NO: 179.

SEQ ID NO: 181 sets forth the nucleotide sequence of the coding region of the cDNA of Dk_06_36, an NLR from Aegilops searsii. If desired, a stop codon (e.g. TAA, TAG, or TGA) can be operably linked to the 3′ end of a nucleic acid molecule comprising or consisting of this NLR sequence. The native stop codon of this cDNA is TGA.

SEQ ID NO: 182 sets forth the amino acid sequence of the NLR protein encoded by SEQ ID NO: 181.

SEQ ID NO: 183 sets forth the nucleotide sequence of the coding region of the cDNA of Dk_08_16, an NLR from Aegilops sharonensis. If desired, a stop codon (e.g. TAA, TAG, or TGA) can be operably linked to the 3′ end of a nucleic acid molecule comprising or consisting of this NLR sequence. The native stop codon of this cDNA is TGA.

SEQ ID NO: 184 sets forth the amino acid sequence of the NLR protein encoded by SEQ ID NO: 183.

SEQ ID NO: 185 sets forth the nucleotide sequence of the coding region of the cDNA of Dk_08_79, an NLR from Avena abyssinica. If desired, a stop codon (e.g. TAA, TAG, or TGA) can be operably linked to the 3′ end of a nucleic acid molecule comprising or consisting of this NLR sequence. The native stop codon of this cDNA is TAG.

SEQ ID NO: 186 sets forth the amino acid sequence of the NLR protein encoded by SEQ ID NO: 185.

SEQ ID NO: 187 sets forth the nucleotide sequence of the coding region of the cDNA of Dk_09_55, an NLR from Briza media. If desired, a stop codon (e.g. TAA, TAG, or TGA) can be operably linked to the 3′ end of a nucleic acid molecule comprising or consisting of this NLR sequence. The native stop codon of this cDNA is TGA.

SEQ ID NO: 188 sets forth the amino acid sequence of the NLR protein encoded by SEQ ID NO: 187.

### DETAILED DESCRIPTION OF THE INVENTION

The present inventions now will be described more fully hereinafter with reference to the accompanying drawings, in which some, but not all embodiments of the inventions are shown. Indeed, these inventions may be embodied in many different forms and should not be construed as limited to the embodiments set forth herein; rather, these embodiments are provided so that this disclosure will satisfy applicable legal requirements. Like numbers refer to like elements throughout.

Many modifications and other embodiments of the inventions set forth herein will come to mind to one skilled in the art to which these inventions pertain having the benefit of the teachings presented in the foregoing descriptions and the associated drawings. Therefore, it is to be understood that the inventions are not to be limited to the specific embodiments disclosed and that modifications and other embodiments are intended to be included within the scope of the appended claims. Although specific terms are employed herein, they are used in a generic and descriptive sense only and not for purposes of limitation.

In one aspect, the present invention relates to methods for preparing a library of candidate plant disease resistance (NLR) genes. Such a library of candidate NLR genes finds use in the increasing the efficiency of methods for the identification of an NLR gene in a plant that is capable of conferring to a susceptible host plant resistance to a plant pathogen of interest. Because plant genomes typically comprise hundreds of NLRs, it can be an arduous task to identify a those NLR genes in plant that provide resistance to plant disease caused by a plant pathogen of interest. The methods of present invention find use in reducing the number of candidate NLR genes using a novel signature that need to be tested in a susceptible host plant to determine if a particular candidate NLR gene is capable of conferring to the susceptible host plant resistance to the plant pathogen of interest. The methods of the present invention involve selecting NLRs that display the signature of high expression in unchallenged plant tissues. This signature has previously been overlooked as NLRs are typically thought to be low expressed genes that can sometimes cause yield penalties. See Lai & Eulgem, 2018, Mol. Plant Pathol. 19(5): 1267-1281; Tian et al., 2003, Nature 423(6935): 74-77; Fitzgerald et al., 2004, MPMI 17(2): 140-151; Chern et al., 2005, MPMI 18(6):511-520; Karasov et al., 2017, Plant Cell 29(4): 666-680; Jones & Dangl, 2006, Nature 444(7117):323-329; Richard et al., 2018, Mol. Plant Pathol. 19(11):2516-2523; and Baggs et al., 2017, Curr. Opin. Plant Biol. 38:59-67.

In a second aspect, the present invention relates to improved methods for identifying an NLR gene against a plant pathogen of interest using a library of candidate NLR genes prepared according to methods of present invention. The methods find use in the identification of new NLR genes that can be incorporated into a crop plant to confer resistance to a plant disease of interest. Such new NLR genes are desired by plant breeders to aid in the development of new crop plant varieties with enhanced resistance to one or more plant diseases.

The methods of present invention find use in identifying NLR genes against a wide range of pathogens including, but not limited to, fungal, bacterial, oomycete, nematode, and viral plant pathogens. Plant pathogens of interest are those plant pathogens that are capable of causing plant disease symptoms on a host plant of interest, particularly a crop plant or other plant grown by humans for food, fiber, or animal feed, more particularly a crop plant or other plant this is known to suffer agronomic yield losses due to plant disease caused by the plant pathogen of interest.

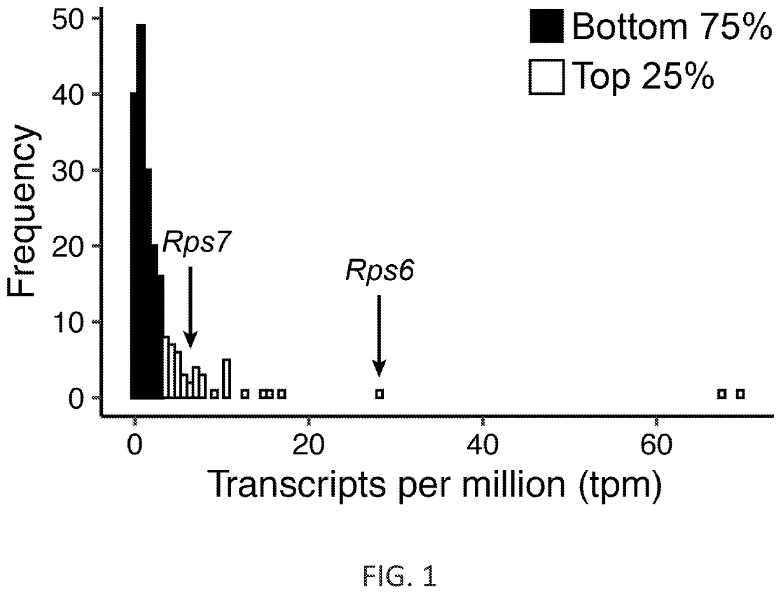

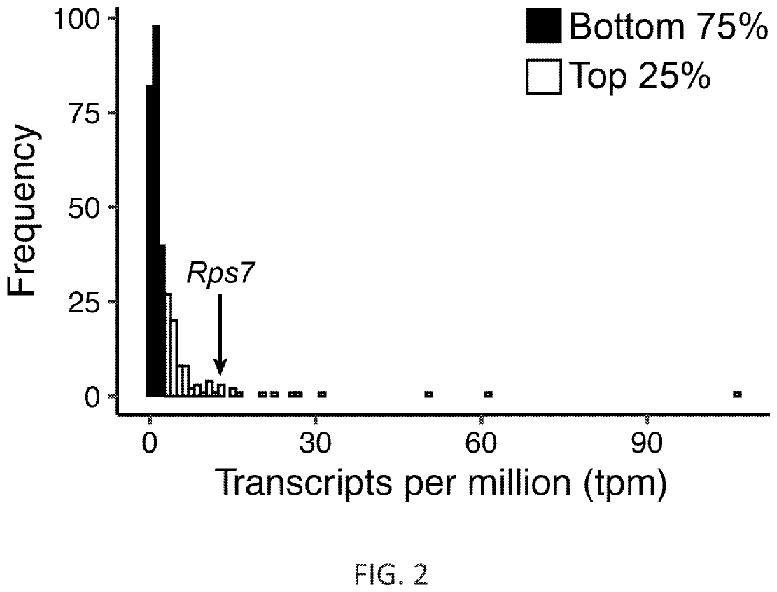

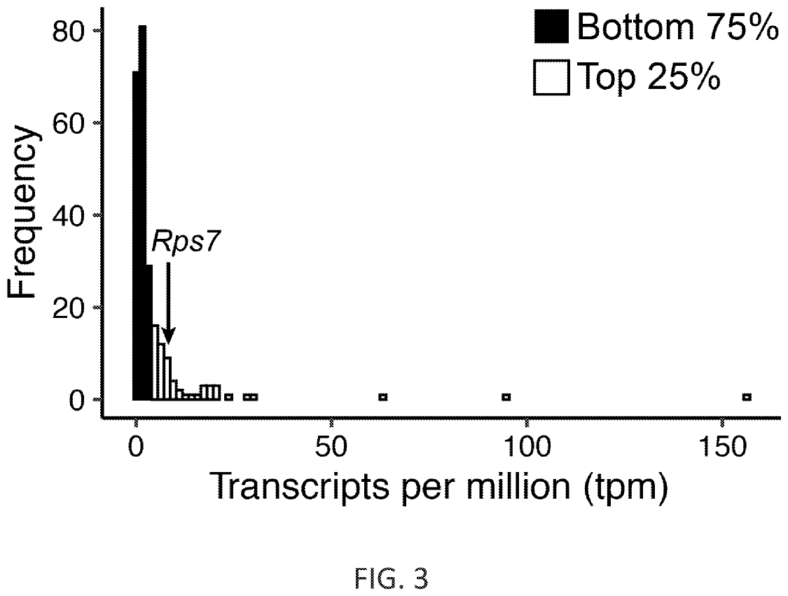

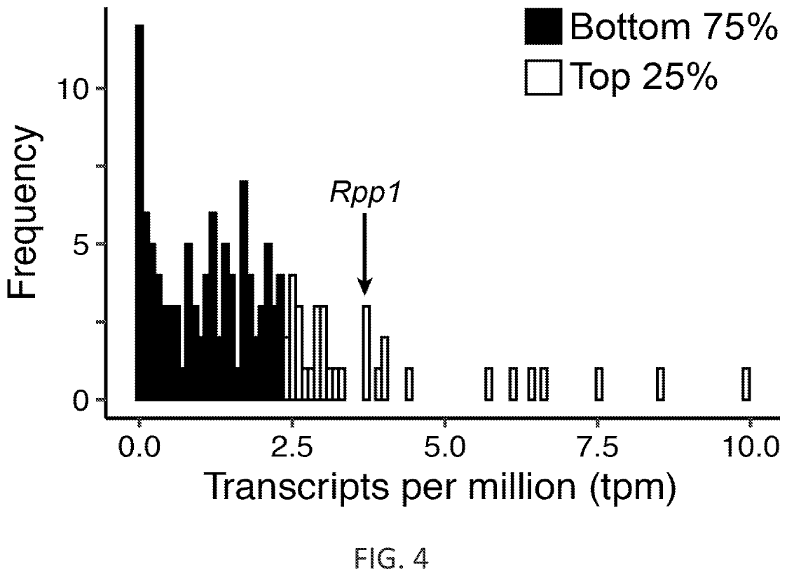

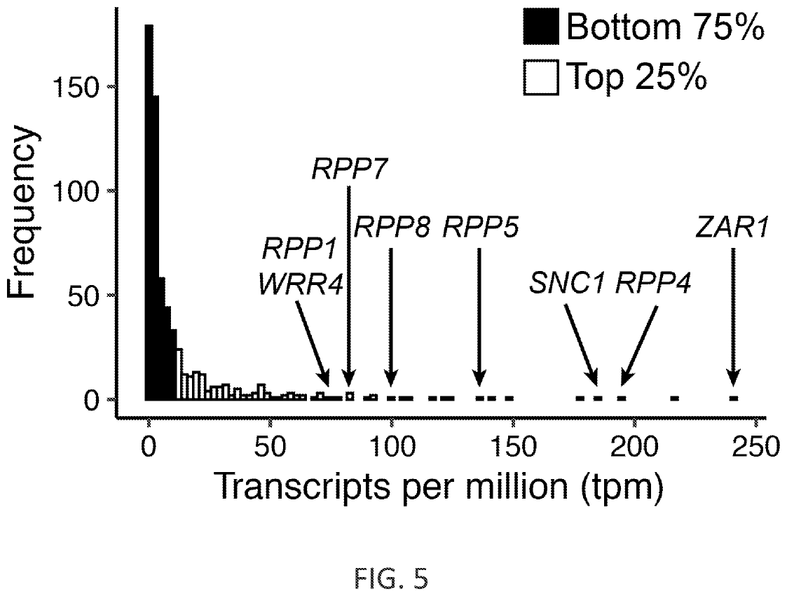

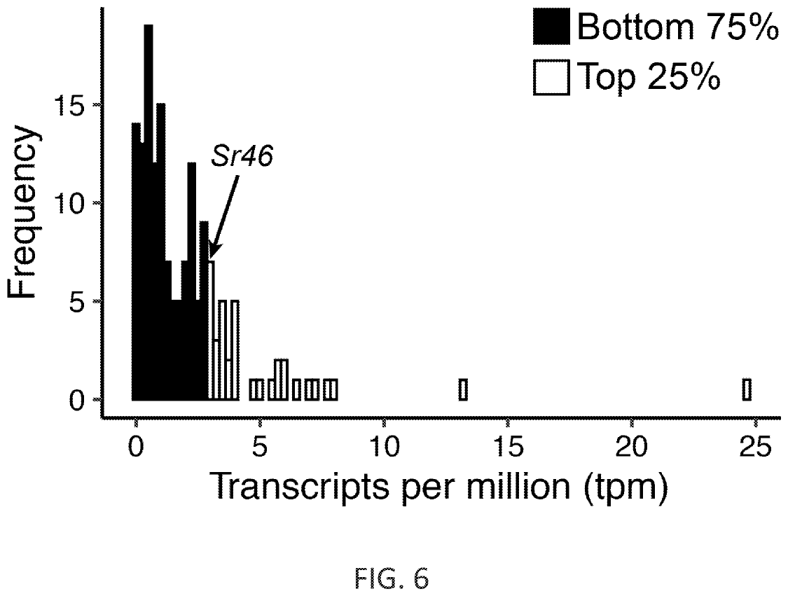

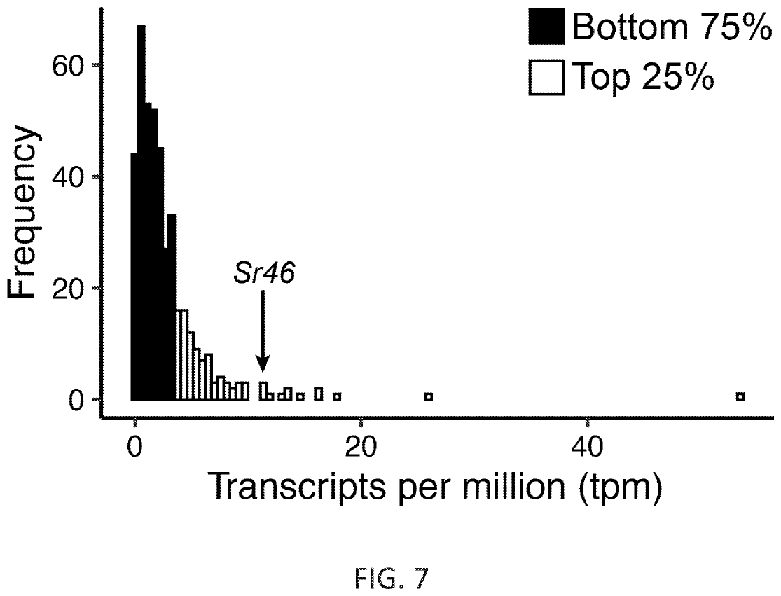

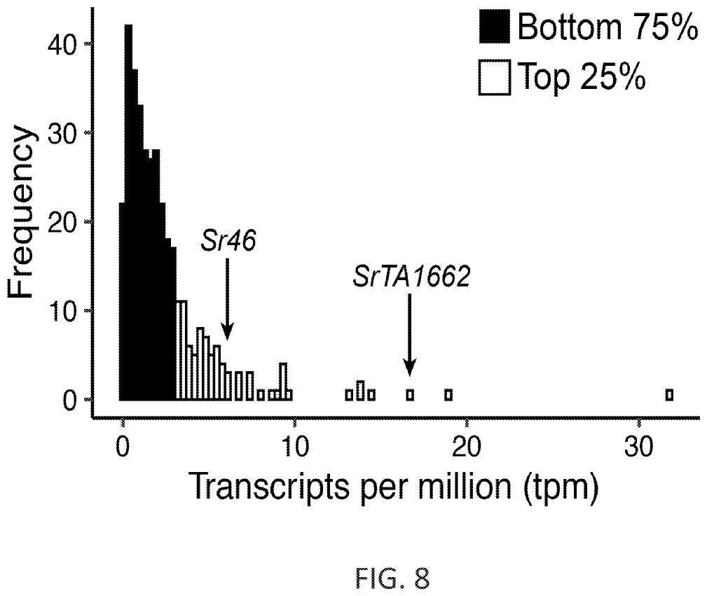

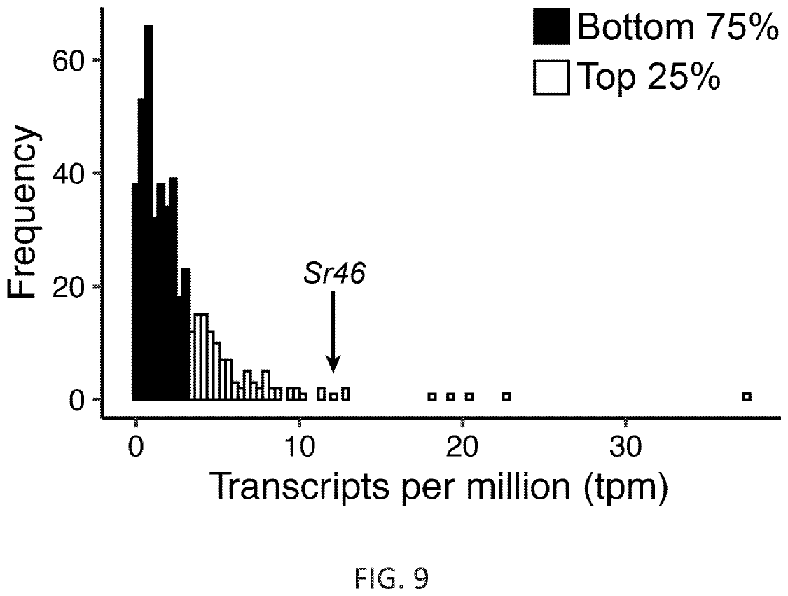

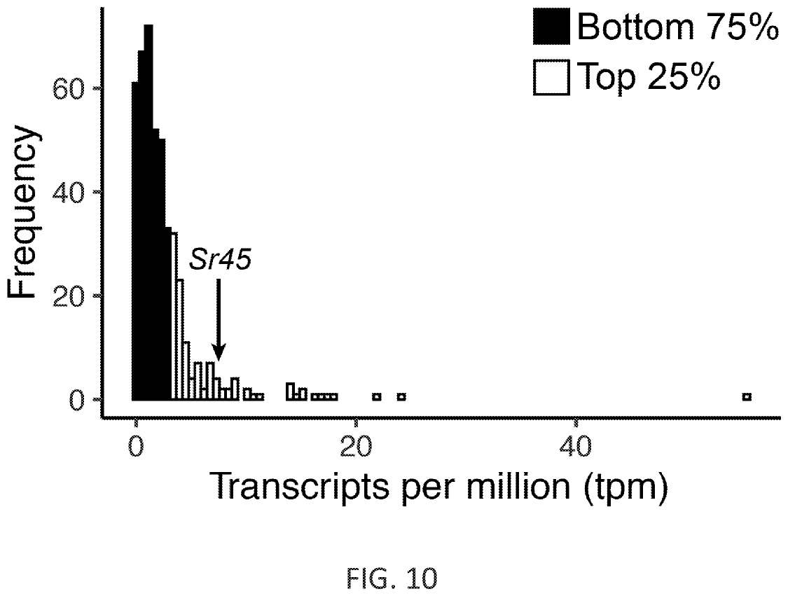

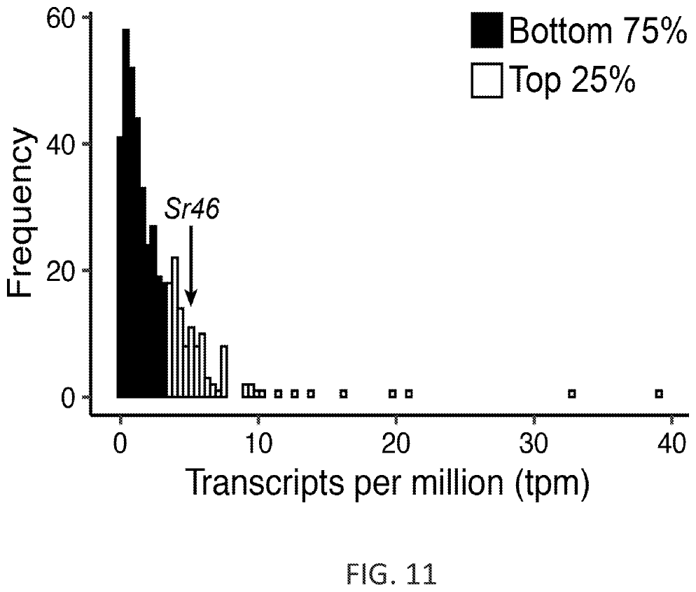

The present invention is based in part on certain observations or discoveries made by the present inventors. First, all characterized NLR genes to foliar pathogens are expressed in unchallenged leaf tissue in monocots and dicots. Published examples include Pm3b, Rpg5, Sr33, and CcRpp1 (Kawashima et al., 2016, Nature Biotechnol. 2016 34(6):661-665; U.S. Pat. No. 10,842,097). Second, the average number of NLRs expressed in a leaf transcriptome is between 100 and 200 for diverse grass species including, but not limited to, wheat, barley, Aegilops sharonensis, Achnatherum hymenoides, Aegilops bicornis, Aegilops longissima, Aegilops searsii, Aegilops sharonensis, Agropyron cristatum, Avena abyssinica, Brachypodium distachyon, Briza media, Cynosurus cristatus, Echinaria capitata, Holcus lanatus, Hordeum vulgare, Koeleria macrantha, Lolium perenne, Melica ciliate, Phalaris coerulescens, and Poa trivialis. This is a fraction of the total number of NLRs in a genome. For example, only about 10% of all the NLRs encoded on the barley/wheat genome are expressed in leaf tissue (FIGS. 1-3). Critically, within the group of NLRs that are expressed in a leaf transcriptome, the subgroup of highly expressed NLRs is saturated for functional R genes (FIGS. 1-11). This discovery is based on performed bioinformatic analyses on the expression level within model species Arabidopsis thaliana, accession Columbia-0 (Col-0), where many R genes have been cloned and characterized. The observations in this well-studied species corroborate our initial observations; of the 10 NLRs that are described to convey resistance, 9 are present in the top 25% of NLRs expressed in leaf tissue (FIG. 5). This signature has been previously overlooked as earlier publications suggested that NLRs have a negative yield impact, leading to the widely held assumption that functional NLRs within this class of protein must be present at low level. The highest expressed NLRs are those which are effective against Hyaloperonospora arabidopsis and Albugo candida which are pathogens that are known to co-evolve with A. thaliana. Publicly available data (Kawashima et al., 2016, Nature Biotechnol. 2016 34(6):661-665) was used to determine if an NLR gene that was identified via map-based cloning (CcRpp1) could be identified using the above criteria. Indeed, CcRpp1 was determined to be in the top 10% of highly expressed NLRs (FIG. 4).

The present invention provides methods for preparing a library of candidate NLR genes against one or more plant pathogen(s) of interest. The methods comprise selecting from each of one or more plants of interest, a subpopulation of highly expressed NLRs from among a population of constitutively expressed NLRs in an organ or other part of the one or more plants, so as to produce a library of candidate R genes. The subpopulation of highly expressed NLRs comprises NLRs that are highly expressed, constitutively in an organ or other part of the plant in the absence of the plant or any organ or other part thereof being contacted with or otherwise exposed to one or more plant pathogens of interest. Such plant tissue is referred to herein as “unchallenged” plant tissue because neither the plant tissue nor any part of the plant from the tissue originates or originated was contacted intentionally with any plant pathogen of interest or is otherwise known to be infected with a plant pathogen or afflicted by any other plant pest such as, for example, insects and mites.

Such unchallenged plant tissue can be a plant organ (e.g. a leaf, a stem, or a root) or any other part of a plant that has not been contacted with or otherwise exposed to a pathogen of interest. Preferably, neither the unchallenged plant tissue nor any other part of the plant has been exposed to the plant pathogen of interest, and the plant is good health and not displaying any symptoms of plant disease or signs of damage from other plant pests such as, for example, insects.

The subpopulation of NLRs that is expressed in plant organ or other part of the plant or plants can be determined by detecting mRNAs of individual NLRs preferably by a transcriptome profiling method such as, for example, RNA Sequencing (RNAseq), which can be used not only to identify of individual NLR genes that are expressed in a plant organ or other part of the plant or plants, but also to assess relative expression levels of the various expressed NLR genes. Thus, RNAseq can be employed to determine both the subpopulation of expressed NLRs in a plant organ or other plant tissue and the portion of the expressed NLRs that are highly expressed candidate R genes to produce the library of candidate R gene. Other methods to identify highly expressed NLRs are those that can be used in the methods of the present invention to quantify differential levels in transcripts including, for example, microarray technologies such as Affymetrix arrays and spotted cDNA arrays. Alternatively, as highly expressed NLRs can be identified by higher average protein levels for the NLR proteins encoded by their respective NLRs, protein quantification methods can be employed including but not limited to LC-MS, LC-MS/MS, MassSpec, Q-TOF, and the like.

Typically, the highly expressed NLRs comprise a relative expression levels in the organ or other part of the plant that is greater than the relative expression levels in the same organ or same part of the plant of at least about 65% of expressed NLRs. Preferably, the highly expressed NLRs comprise expression levels in the organ or other part of the plant this is greater than the relative expression levels in the same organ or same part of the plant of at least about 65%, 70%, 75%, 80%, 85%, 90%, or 95% of expressed NLRs. In other words, the highly expressed NLRs in a particular organ or other part of a plant of interest are those expressed NLRs that have expression levels in at least about the top 35%, 30%, 25%, 20%, 15%, 10%, 5%, 4%, or 3%, when compared to the expression levels of all expressed NLRs in the particular organ or other part of the plant of interest. Preferably, the highly expressed NLRs in a particular organ or other part of a plant of interest are those expressed NLRs that have expression levels in at least about the top 25%, 20%, 15%, 10%, 5%, 4%, or 3%, when compared to the expression levels of all expressed NLRs in the particular organ or other part of the plant of interest. More preferably, the highly expressed NLRs in a particular organ or other part of a plant of interest are those expressed NLRs that have expression levels in at least about the top 20%, 15%, 10%, 5%, 4%, or 3%, when compared to the expression levels of all expressed NLRs in the particular organ or other part of the plant of interest. Most preferably, the highly expressed NLRs in a particular organ or other part of a plant of interest are those expressed NLRs that have expression levels in at least about the top 20%, 15%, 10%, 5%, 4%, or 3%, when compared to the expression levels of all expressed NLRs in the particular organ or other part of the plant of interest.

It is recognized that the choice of any suitable relative expression level for determining the highly expressed NLRs will depend on any number of factors including, for example, the plant species, the plant organ or other part of the plant used as the mRNA source, the total number of expressed NLR genes in the plant of interest, the portion of the total NLRs in the genome of the plant that are expressed in the plant organ or other plant part, and the growth conditions of the plant from which the mRNA was isolated.

In certain embodiments of the invention involving RNAseq, TransDecoder (v4.1.0; available on the World Wide Web at github.com/TransDecoder/TransDecoder/releases) LongOrfs can be used to predict all open reading frames in de novo assembled transcriptomes. To identify transcripts encoding putative NLR proteins, InterProScan (v5.27-66.0) (Jones et al., (2014) Bioinformatics 30(9): 1236-1240; doi: 10.1093/bioinformatics/btu031) can be used, for example, to annotate domains using Coils and the Pfam, Superfamily, and ProSite databases. Any NLR gene encoding a protein containing both a nucleotide binding (NB) domain and a leucine-rich repeat (LRR) domain can be identified as an NLR protein and advanced in the analysis. A custom script developed from FAT-CAT (Afrasiabi et al. (2013) Nucleic Acids Res. 41:W242-W248, doi.org/10.1093/nar/gkt399) can be used to classify nucleotide binding domains based on a phylogenetic tree developed from rice, Brachypodium distachyon, and barley nucleotide binding domains derived from NLRs. NLR encoding genes can be advanced, for example, based on the following requirements: the transcript contains either a complete or a 5′ partial open reading frame; the gene is among the top 25% expressed NLRs in the plant organ or other plant part; and the gene does not belong to NLR families known to require an additional NLR (see, for example, Bailey et al. (2018) Genome Biol. 19:23). Among the candidate NLRs, redundancy was removed using CD-HIT (v4.7) requiring 100% identity (-c 1.0). PCR primers were developed using Gateway adapters attB1 (SEQ ID NO: 27) and attB2 (SEQ ID NO: 28) fused to first 20 nucleotides of the start or end of the coding sequence, respectively. See Katzen. (2007) Expert Opin. Drug Discov. 2(4):571-589 for an overview of the Gateway cloning technology.

In this embodiment of the invention, the identified NLR proteins comprise at least one NB domain and at least one LRR domain. Such identified NLR proteins can further comprise one or more additional domains, particularly domains that are known to occur in NLR proteins including, but not limited to, a coiled-coiled (CC) domain, a Toll/Interleukin-1 Receptor (TIR) domain, an additional NB domain, and an additional LRR domain. Examples of identified NLR proteins of the present invention are further described in Example 2 below.

While the typical order for the domains of known NLR proteins in an N-terminal to C-terminal direction is CC-NB-LRR, TIR-NB-LRR, or NB-LRR, the methods of the present invention do not depend on NLR proteins having particular structure and can accommodate domain structures that are atypical for known NLR proteins.

In certain embodiment of the invention, the methods for preparing a library of candidate NLR genes against at least one plant pathogen of interest can comprise a further selection for NLRs comprising at least one additional feature of interest, whereby the library of candidate NLR genes comprises those NLRs that are highly expressed and comprise the one of more additional features of interest. Previous work has established molecular and evolutionary signatures of NLRs that contribute to plant immunity such as gene family and rapid evolution (Yang et al., 2013, PNAS 110:18572-18577). Such features of interest include, but are not limited to:
(i) the presence of intraspecific variation in the amino acid sequence encoded by an NLR;
(ii) the absence of intraspecific variation in the amino acid sequence encoded by an NLR;
(iii) the presence of interspecific variation in the amino acid sequence encoded by an NLR;
(iv) the absence of interspecific variation in the amino acid sequence encoded by an NLR; and
(v) substantial interspecific allelic variation in the amino acid sequence encoded by an NLR.

Unless stated otherwise or apparent from the context of a use “substantial intraspecific and interspecific variation” for the present invention is intended to mean the presence of maintained sequence polymorphisms, diversifying selection, and the over-representation of nonsynonymous substitutions as compared to synonymous substitutions present in alleles maintained across individuals within a population. Examples of NLRs with substantial intraspecific allelic variation include the Mla alleles in barley (Jørgensen, 1994, Plant Sci. 13:97-119; Seeholzer et al., 2010, MPMI 23:497-509) and Pm3 alleles in wheat (Bourras et al., 2018, Curr. Opin. Microbiol. 46:26-33; Bourras et al., 2015, Bourras et al., 2015, Plant Cell 27:2991-3012)

The methods of present invention comprise selecting the NLRs that are highly expressed in an organ or other part of the plant(s) of interest so as to produce a library of candidate NLR genes. Plants of interest include, for example, crop plants and both domesticated and non-domesticated relatives of crop plants. Such relatives include plants that are from same species as the crop plant or relatives that are different species as the crop but are from the same family, subfamily, and/or tribe as the crop plant. In some embodiments of the invention, the plant from which the library of candidate NLR genes is derived is a non-domesticated relative of a host plant that is a crop plant and the candidate NLR gene are intended for use in the crop plant. Preferably, such relatives of a host plant are in the same family, subfamily, tribe, and/or genus as the plant from which the library of candidate NLR genes is derived. In some other embodiments, the host plant and the plant from which the library of candidate NLR genes is derived are from the same species.

A plant or plants of interest from which the library of candidate NLR genes is derived can be any plant accession, variety, or species that does not support growth or lifecycle completion of a pathogen of interest. Indeed, an R gene that is derived from a plant of interest of a first species can be transferred into a plant of a second species that is a host of for plant pathogen of interest whereby a resistant plant of the second species is produced. Examples of R genes that are derived from one species and transferred into a second species include, but are not limited to, the NLRs Bs2 from pepper (Capsicum annuum) (Tai et al., 1999, PNAS 96(24): 14153-14158; transferred into tomato, i.e. Solanum lycopersicum) and CcRpp1 from pigeon pea (Cajanus cajan) (Kawashima et al., 2016, Nature Biotechnol. 2016 34(6):661-665; transferred into soybean, i.e. Glycine max). Preferably for the present invention, the first and second species are in the same family. In certain embodiments, the first and second species are in the same family, but in a different subfamily, tribe, and/or genus.

In certain preferred embodiments, plants that are expected to comprise one or more effective NLR resistance genes against one or more pathogens of interest are used as the plants from which libraries of NLR genes are derived. Such plants are expected to comprise effective NLR resistance genes against one or more pathogens of interest because the plants do not support the growth of the one or more plant pathogens of interest. For the example, relatives of bread wheat (T. aestivum) that such carry effective resistance against one or more pathogens of wheat are species in the Poaceae family including, but not limited to, species in the genera Achnatherum, Aegilops, Agropyron, Avena, Brachypodium, Briza, Cynosurus, Echinaria, Holcus, Hordeum, Koeleria, Lolium, Melica, Phalaris, and Poa. Such species include, for example, Achnatherum hymenoides, Aegilops bicornis, Aegilops longissima, Aegilops searsii, Aegilops sharonensis, Agropyron cristatum, Avena abyssinica, Brachypodium distachyon, Briza media, Cynosurus cristatus, Echinaria capitata, Holcus lanatus, Hordeum vulgare, Koeleria macrantha, Lolium perenne, Melica ciliata, Phalaris coerulescens, and Poa trivialis.

A library of candidate R genes of the present invention can be produced using one or more plants of interest, wherein each of the plants is genetically distinct from one another. If, for example, a library of candidate R genes can be produced using two, three, four, or more plants of interest from the same species, such two, three, four, or more plants of interest can have the same genotypes or two, three, four, or more different genotypes. It is recognized that the number of plants of interest used to produce a library of candidate R genes can vary depending on a number of factors, including, for example, the host plant, the pathogen or pathogens of interest, and the availability of genetically distinct plants of interest that are expected to comprise effective NLR genes against the one or more plant pathogens. Thus, using the methods of the present invention, a library of candidate R genes can be produced using at least 1, 2, 3, 4, 5, 10, 15, 20, 25, 30, 40, 50, 60, 70, 80, 90, 100, 125, 150, 200, 250, 300, 400, 500, 600, 700, 800, 900, 1000, 1250, 1500, 1750, 2000, or more genetically distinct plants of interest.

The methods of the present invention do not depend upon use of a particular plant organ or plant part. Any plant organ or plant part at any developmental stage and/or grown under any environmental conditions, notwithstanding that the plant organ or plant part is from an unchallenged plant. Plant organs include, but not are not limited to, leaves, stems, flowers, roots, fruits, pods, seeds, cotyledons, hypocotyls, epicotyls, radicles, and the like. Plant parts include, for example, leaf midribs, leaf blades, petals, sepals, pedicles, peduncles, and internodes. In certain embodiments of the invention that are described in detail below, the plant organ is a leaf.

The present invention further provides compositions comprising a library of candidate NLR genes produced according to methods described above. Such a library comprises at least two candidate NLR genes but typically comprises at least about 10, 20, 30, 40, 50, 75, 100, 125, 150, 175, 200, 225, 250, 300, 400, 500, 600, 700, 800, 900, 1000, 1250, 1500, 1750, 2000, or more candidate NLR genes. Such compositions find use in methods for identifying a plant disease resistance (NLR) gene against a plant pathogen of interest.

Further provided are compositions comprising a collection of transgenic plants, wherein each of the transgenic plants is produced by transforming a host plant with a candidate NLR gene from a library of candidate NLR genes prepared according to the methods described above. Such compositions also find use in methods for identifying a plant disease resistance (NLR) gene against a plant pathogen of interest. A collection of transgenic plants of the present invention comprises at least two transgenic plants but typically comprises at least about 10, 20, 30, 40, 50, 75, 100, 125, 150, 175, 200, 225, 250, 300, 400, 500, 600, 700, 800, 900, 1000, 1250, 1500, 1750, 2000, or more plants with each transgenic plant comprising a different candidate R gene. Preferably, the collection of transgenic plants comprises transgenic plants representing at least about 50%, 60%, 70%, or 80% of the NLR genes in a library of candidate NLR genes. More preferably, the collection of transgenic plants comprises transgenic plants representing at least about 90%, 95%, 96%, 97%, 98%, 99%, or 100% of the NLR genes in a library of candidate NLR genes. If, for example, a library of candidate NLR genes comprises 99 different NLR genes, a collection of transgenic plants representing all the NLR genes in the library will comprise at least 99 plants, with the each of the 99 plants comprising a different NLR gene. It is recognized that the collection of transgenic plants can comprise two or more transgenic plants for each different NLR gene. The two or more transgenic plants comprising the same NLR gene can comprise in their respective genomes the same transgenic event for which the NLR gene is located in same position in their respective genomes. Alternatively, the two or more transgenic plants comprising the same NLR gene can comprise in their respective genomes independent transgenic events for which the NLR gene is not located in same position in their respective genomes.

The present invention further provides compositions for identifying an NLR gene against a plant pathogen of interest involving the use of a library of candidate NLR genes. Such methods comprise producing a host plant transformed with a candidate NLR gene selected from a library of NLR genes prepared according to the methods of the present invention. The host plant is a host for the plant pathogen of interest and the plant pathogen is capable of causing plant disease symptoms on the host plant under suitable environmental conditions for the development of disease symptoms. The methods further comprise contacting the transformed host plant, or otherwise exposing the transformed host plant to, the plant pathogen of interest under environmental conditions suitable for the development of disease symptoms, and then after a period of time sufficient for the development of disease symptoms, determining if the transformed host plant displays enhanced resistance to the plant pathogen of interest when compared to a control host plant that does not comprise the candidate NLR gene, wherein the candidate NLR gene is an NLR gene against the plant pathogen of interest when the transformed host plant displays enhanced resistance to plant disease symptoms caused by the plant pathogen of interest.

It is recognized that such suitable environmental conditions for the development of disease symptoms depends on the host plant-plant pathogen combination and is known in the art or can be determined using routine methods available in the art. It is further recognized that the period of time after inoculation (i.e. after contacting the host plant with the pathogen) that is sufficient for the development of disease symptoms also depends on the host plant-plant pathogen combination and either is known in the art or can be determined using routine methods available in the art.

The present invention further provides methods for identifying an NLR gene against a plant pathogen of interest involving the use of a transgenic plant comprising a candidate NLR gene from a library of candidate NLR genes prepared according to the methods described above or a collection of such transgenic plants. Such methods comprise contacting the transgenic plant or the collection of transgenic plants with the plant pathogen of interest under environmental conditions suitable for the development of disease symptoms. The transgenic plants are host plants for the plant pathogen of interest and the plant pathogen is capable of causing plant disease symptoms on the host plant. The methods further comprise assessing disease symptoms on the transgenic plant or plants after a period of time sufficient for the development of disease symptoms following contacting the members with the plant pathogen. A transgenic plant comprising an NLR gene against the plant pathogen of interest is identified when the transgenic plant displays enhanced resistance to plant disease caused by the plant pathogen of interest, when compared to a control plant that does not comprise a candidate NLR gene.

A collection of transgenic plants of the present invention is not limited to use with a single pathogen. As described in detail below in the Examples, a collection of transgenic plants can be separately screened for resistance to one, two, three, four, five, or more plant pathogens of interest that are capable of causing plant disease symptoms on the host plant to identify functional NLR genes from among the candidate NLR genes represented in the collection of transgenic plants. Such functional NLR genes are NLR genes that are capable of conferring to a host plant comprising the NLR gene resistance against one or more of the pathogens of interest.

The present invention further relates to nucleic acid molecule compositions comprising isolated NLR genes of the present invention and other nucleic molecules encoding NLR proteins encoded by such NLR genes and to protein compositions comprising NLR proteins of the present invention. Such compositions include, but not limited to, plants, plant cells, and other host cells comprising one or more of such NLR proteins and/or one or more nucleic acid molecules, and expression cassettes and vectors comprising one or more of such nucleic acid molecules.

The present invention encompasses nucleic acid molecules comprising one or more of the nucleotide sequences encoding NLR proteins disclosed herein or in the accompanying sequence listing and/or drawings. Such nucleic acid molecules include, but not limited to, a nucleic acid molecule comprising at least one a nucleotide sequence selected from the group consisting of: the nucleotide sequence set forth in SEQ ID NO: 1, 3, 5, 7, 9, 11, 13, 15, 17, 19, 21, 23, 25, 29, 31, 33, 35, 37, 39, 41, 43, 45, 47, 49, 51, 53, 55, 57, 59, 61, 63, 65, 67, 69, 71, 73, 75, 77, 79, 81, 83, 85, 87, 89, 91, 93, 95, 97, 99, 101, 103, 105, 107, 109, 111, 113, 115, 117, 119, 121, 123, 125, 127, 129, 131, 133, 135, 137, 139, 141, 143, 145, 147, 149, 151, 153, 155, 157, 159, 161, 163, 165, 167, 169, 171, 173, 175, 177, 179, 181, 183, 185, or 187; a nucleotide sequence encoding a polypeptide comprising an amino acid sequence set forth in SEQ ID NO: 2, 4, 6, 8, 10, 12, 14, 16, 18, 20, 22, 24, 26, 30, 32, 34, 36, 38, 40, 42, 44, 46, 48, 50, 52, 54, 56, 58, 60, 62, 64, 66, 68, 70, 72, 74, 76, 78, 80, 82, 84, 86, 88, 90, 92, 94, 96, 98, 100, 102, 104, 106, 108, 110, 112, 114, 116, 118, 120, 122, 124, 126, 128, 130, 132, 134, 136, 138, 140, 142, 144, 146, 148, 150, 152, 154, 156, 158, 160, 162, 164, 166, 168, 170, 172, 174, 176, 178, 180, 182, 184, 186, or 188; a nucleotide sequence set forth in the sequence listing; a nucleotide sequence encoding an amino acid sequence set forth in the sequence listing; and variants thereof. Preferably, such nucleic acid molecules are capable of conferring to a plant, particularly a wheat plant, a barley plant, a triticale plant, and/or an oat plant, enhanced resistance to one or more plant pathogens of interest including, for example, wheat stem rust (Puccinia graminis f. sp. tritici), wheat stripe rust (Puccinia striiformis f. sp. tritici), wheat leaf rust (Puccinia triticina), wheat blast (Magnaporthe oryzae Triticum) and wheat powdery mildew (Blumeria graminis f. sp. tritici).

The present invention further encompasses plants, plant cells, host cells, expression cassettes, polynucleotide constructs and vectors comprising at least one of such nucleic acid molecules, as well as food products produced from such plants. Additionally encompassed by the present invention are uses of plants comprising at least one of such nucleic acid molecules in the methods disclosed elsewhere herein such as, for example, methods of limiting plant diseases in agricultural crop production.

In certain embodiments of present invention, the plants and plant cells of the present invention comprise at least one heterologous polynucleotide construct comprising a nucleic acid of the present invention. Such a heterologous polynucleotide can be introduced into a plant or a cell thereof by a stable or transient plant transformation method disclosed elsewhere herein or otherwise known in the art.

The present invention additionally provides methods for enhancing the resistance of a plant to a plant pathogen, particularly a plant comprising partial resistance to the plant pathogen. As used herein, full or complete resistance is defined as the inability of the pathogen to spread within the host plant genotype. With full resistance, localized cell death is observed on the plant after being contacted by the pathogen but there are no spreading lesions. In contrast with partial resistance, the pathogen may still be able to infect the host plant and cause a spreading lesion, but the spread of the lesion is restricted or limited, when compared to a susceptible plant.

Such methods for enhancing the resistance of a plant comprise modifying a plant cell to be capable of expressing of NLR protein. The methods optionally further comprise regenerating the modified plant cell into a modified plant comprising enhanced resistance to the plant pathogen.

In some embodiments, the methods comprise introducing into at least one plant cell a polynucleotide construct comprising an NLR gene of the present invention with its native promoter. In other embodiments, such methods comprise introducing into at least one plant cell a polynucleotide construct comprising a promoter that drives expression in a plant and an operably linked nucleic acid molecule encoding the NLR protein using plant transformation methods described elsewhere herein or otherwise known in the art. Preferred promoters for enhancing the resistance of a plant to a plant pathogen are promoters known to drive high-level gene expression such as, for example, the CaMV 35S promoter and the maize ubiquitin promoter. Additional promoters that are suitable for use in the methods of the present invention are described hereinbelow.

The methods of the present invention find use in producing plants with enhanced resistance to a plant disease caused by a plant pathogen. Typically, the methods of the present invention will enhance or increase the resistance of the subject plant to one strains of a plant pathogen or to each of two or more strains of the plant pathogen by at least 25%, 50%, 75%, 100%, 150%, 200%, 250%, 500% or more when compared to the resistance of a control to same strain or strains of the plant pathogen. Unless stated otherwise or apparent from the context of a use, a control plant for the present invention is a plant that does not comprise the polynucleotide construct of the present invention. Preferably, the control plant is essentially identical (e.g. same species, subspecies, and variety) to the plant comprising the polynucleotide construct of the present invention except that the control does not comprise the polynucleotide construct. In some embodiments, the control plant will comprise a polynucleotide construct but not comprise a candidate NLR gene or NLR gene of the present invention or a nucleotide sequence encoding a protein that is encoded by such a candidate NLR gene or NLR gene. In other embodiments, the control plant will not comprise a polynucleotide construct.

The plants of the present invention comprising an NLR gene disclosed herein find use in methods for limiting plant disease caused by at least one plant pathogen in agricultural crop production, particularly in regions where such a plant disease is prevalent and is known to negatively impact, or at least has the potential to negatively impact, agricultural yield. The methods of the invention comprise planting a plant (e.g. a seedling), seed, or tuber of the present invention, wherein the plant, seed, or tuber comprises at least one NLR gene of the present invention. The methods further comprise growing the plant that is derived from the seedling, seed, or tuber under environmental conditions favorable for the growth and development of the plant, and optionally harvesting at least one fruit, tuber, leaf, or seed from the plant. Such environmental conditions can include, for example, air temperature, soil temperature, soil water content, photoperiod, light intensity, soil pH, and soil fertility. It is recognized that the environmental conditions favorable for the growth and development of a plant of interest will vary depending on, for example, the plant species or even the particular variety (e.g. cultivar) or genotype of the plant of interest. It is further recognized that the environmental conditions that are favorable for the growth and development of the plants of interest of the present invention are known in the art.

Additionally, the present invention provides plants, seeds, and plant cells produced by the methods of present invention and/or comprising a polynucleotide construct of the present invention. Also provided are progeny plants and seeds thereof comprising a polynucleotide construct of the present invention. The present invention also provides seeds, vegetative parts, and other plant parts produced by the transformed plants and/or progeny plants of the invention as well as food products and other agricultural products produced from such plant parts that are intended to be consumed or used by humans and other animals including, but not limited to pets (e.g., dogs and cats) and livestock (e.g., pigs, cows, chickens, turkeys, and ducks).

The present invention encompasses isolated or substantially purified polynucleotide (also referred to herein as “nucleic acid molecule”, “nucleic acid” and the like) or protein (also referred to herein as “polypeptide”) compositions including, for example, polynucleotides and proteins comprising the sequences set forth in the accompanying Sequence Listing as well as variants and fragments of such polynucleotides and proteins. An “isolated” or “purified” polynucleotide or protein, or biologically active portion thereof, is substantially or essentially free from components that normally accompany or interact with the polynucleotide or protein as found in its naturally occurring environment. Thus, an isolated or purified polynucleotide or protein is substantially free of other cellular material or culture medium when produced by recombinant techniques, or substantially free of chemical precursors or other chemicals when chemically synthesized. Optimally, an “isolated” polynucleotide is free of sequences (optimally protein encoding sequences) that naturally flank the polynucleotide (i.e., sequences located at the 5′ and 3′ ends of the polynucleotide) in the genomic DNA of the organism from which the polynucleotide is derived. For example, in various embodiments, the isolated polynucleotide can contain less than about 5 kb, 4 kb, 3 kb, 2 kb, 1 kb, 0.5 kb, or 0.1 kb of nucleotide sequence that naturally flank the polynucleotide in genomic DNA of the cell from which the polynucleotide is derived. A protein that is substantially free of cellular material includes preparations of protein having less than about 30%, 20%, 10%, 5%, or 1% (by dry weight) of contaminating protein. When the protein of the invention or biologically active portion thereof is recombinantly produced, optimally culture medium represents less than about 30%, 20%, 10%, 5%, or 1% (by dry weight) of chemical precursors or non-protein-of-interest chemicals.

Fragments and variants of the disclosed polynucleotides and proteins encoded thereby are also encompassed by the present invention. By “fragment” is intended a portion of the polynucleotide or a portion of the amino acid sequence and hence protein encoded thereby. Fragments of polynucleotides comprising coding sequences may encode protein fragments that retain biological activity of the full-length or native protein. Alternatively, fragments of a polynucleotide that are useful as hybridization probes generally do not encode proteins that retain biological activity or do not retain promoter activity. Thus, fragments of a nucleotide sequence may range from at least about 20 nucleotides, about 50 nucleotides, about 100 nucleotides, and up to the full-length polynucleotide of the invention.

“Variants” is intended to mean substantially similar sequences. For polynucleotides, a variant comprises a polynucleotide having deletions (i.e., truncations) at the 5′ and/or 3′ end; deletion and/or addition of one or more nucleotides at one or more internal sites in the native polynucleotide; and/or substitution of one or more nucleotides at one or more sites in the native polynucleotide. As used herein, a “native” polynucleotide or polypeptide comprises a naturally occurring nucleotide sequence or amino acid sequence, respectively. For polynucleotides, conservative variants include those sequences that, because of the degeneracy of the genetic code, encode the amino acid sequence of the proteins of the NLR genes of the present invention. Variant polynucleotides include synthetically derived polynucleotides, such as those generated, for example, by using site-directed mutagenesis but which still encode a functional NLR protein of the invention. Generally, variants of a polynucleotide of the invention will have at least about 80%, 85%, 90%, 91%, 92%, 93%, 94%, 95%, 96%, 97%, 98%, 99% or more sequence identity to that polynucleotide as determined by sequence alignment programs and parameters as described elsewhere herein.

Variants of a polynucleotide of the invention (i.e., the reference polynucleotide) can also be evaluated by comparison of the percent sequence identity between the polypeptide encoded by a variant polynucleotide and the polypeptide encoded by the reference polynucleotide. Percent sequence identity between any two polypeptides or between the corresponding parts (e.g. domains) of any two peptides can be calculated using sequence alignment programs and parameters described elsewhere herein. Where any given pair of polynucleotides of the invention or corresponding parts thereof is evaluated by comparison of the percent sequence identity shared by the two polypeptides they encode, the percent sequence identity between the two encoded polypeptides is at least about 50%, 55%, 60%, 65%, 70%, 75%, 80%, 85%, 86%, 87%, 88%, 89%, 90%, 91%, 92%, 93%, 94%, 95%, 96%, 97%, 98%, 99% or more sequence identity.

“Variant” protein is intended to mean a protein derived from the native protein by deletion (so-called truncation) of one or more amino acids at the N-terminal and/or C-terminal end of the native protein; deletion and/or addition of one or more amino acids at one or more internal sites in the native protein; or substitution of one or more amino acids at one or more sites in the native protein. A biologically active variant of a protein of the invention may differ from that protein by as few as 1-15 amino acid residues, as few as 1-10, such as 6-10, as few as 5, as few as 4, 3, 2, or even 1 amino acid residue. Biologically active variants of an NLR protein of the present invention will have at least about 80%, 85%, 86%, 87%, 88%, 89%, 90%, 91%, 92%, 93%, 94%, 95%, 96%, 97%, 98%, 99% or more sequence identity to the amino acid sequence of NLR protein of the present invention as determined by sequence alignment programs and parameters described elsewhere herein. A biologically active variant of an NLR protein of the invention or of a domain thereof may differ from that protein or domain by as few as 1-15 amino acid residues, as few as 1-10, such as 6-10, as few as 5, as few as 4, 3, 2, or even 1 amino acid residue.

The proteins of the invention may be altered in various ways including amino acid substitutions, deletions, truncations, and insertions. Methods for such manipulations are generally known in the art. Methods for mutagenesis and polynucleotide alterations are well known in the art. See, for example, Kunkel (1985) Proc. Natl. Acad. Sci. USA 82:488-492; Kunkel et al. (1987) Methods in Enzymol. 154:367-382; U.S. Pat. No. 4,873,192; Walker and Gaastra, eds. (1983) Techniques in Molecular Biology (MacMillan Publishing Company, New York) and the references cited therein. Guidance as to appropriate amino acid substitutions that do not affect biological activity of the protein of interest may be found in the model of Dayhoff et al. (1978) Atlas of Protein Sequence and Structure (Natl. Biomed. Res. Found., Washington, D.C.), herein incorporated by reference. Conservative substitutions, such as exchanging one amino acid with another having similar properties, may be optimal.

The deletions, insertions, and substitutions of the protein sequences encompassed herein are not expected to produce radical changes in the characteristics of the protein. However, when it is difficult to predict the exact effect of the substitution, deletion, or insertion in advance of doing so, one skilled in the art will appreciate that the effect will be evaluated by routine screening assays. That is, the activity can be evaluated by, for example, assays for disease resistance against a plant pathogen of interest as disclosed elsewhere herein or otherwise known in the art.

For example, a plant that is susceptible to a plant disease caused by a plant pathogen of interest can be transformed with a polynucleotide construct comprising an NLR gene of the present invention, regenerated into a transformed or transgenic plant comprising the polynucleotide constructs, and tested for resistance using standard disease resistance assays known in the art or described elsewhere herein.

Variant polynucleotides and proteins also encompass sequences and proteins derived from a mutagenic and recombinogenic procedure such as DNA shuffling. Strategies for such DNA shuffling are known in the art. See, for example, Stemmer (1994) Proc. Natl. Acad. Sci. USA 91:10747-10751; Stemmer (1994) Nature 370:389-391; Crameri et al. (1997) Nature Biotech. 15:436-438; Moore et al. (1997) J. Mol. Biol. 272:336-347; Zhang et al. (1997) Proc. Natl. Acad. Sci. USA 94:4504-4509; Crameri et al. (1998) Nature 391:288-291; and U.S. Pat. Nos. 5,605,793 and 5,837,458.

Preferably, the NLR genes of the present invention and the polynucleotides encoding them confer, or are capable of conferring, upon a plant comprising such an NLR gene, enhanced resistance to at least one plant pathogen, but preferably to two, three, four, five, or more plant pathogens.

PCR amplification can be used in certain embodiments of the methods of the present invention. Methods for designing PCR primers and PCR amplification are generally known in the art and are disclosed in Sambrook et al. (1989) Molecular Cloning: A Laboratory Manual (2d ed., Cold Spring Harbor Laboratory Press, Plainview, New York). See also Innis et al., eds. (1990) PCR Protocols: A Guide to Methods and Applications (Academic Press, New York); Innis and Gelfand, eds. (1995) PCR Strategies (Academic Press, New York); and Innis and Gelfand, eds. (1999) PCR Methods Manual (Academic Press, New York). Known methods of PCR amplification include, but are not limited to, methods using paired primers, nested primers, single specific primers, degenerate primers, gene-specific primers, vector-specific primers, partially mismatched primers, and the like.

It is recognized that the nucleic acid molecules of the NLR genes of the present invention encompass nucleic acid molecules comprising a variant nucleotide sequence that is sufficiently identical to the nucleotide sequence of an NLR gene of the present invention. The term “sufficiently identical” is used herein to refer to a first amino acid or nucleotide sequence that contains a sufficient or minimum number of identical or equivalent (e.g., with a similar side chain) amino acid residues or nucleotides to a second amino acid or nucleotide sequence such that the first and second amino acid or nucleotide sequences have a common structural domain(s) and/or common functional activity, such as, for example, disease resistance. For example, amino acid or nucleotide sequences that contain a common structural domain(s) and/or sequences having at least about 45%, 55%, or 65% identity, preferably 75% identity, more preferably 85%, 86%, 87%, 88%, 89%, 90%, 95%, 96%, 97%, 98% or 99% identity, can be as sufficiently identical.

To determine the percent identity of two amino acid sequences or of two nucleic acids, the sequences are aligned for optimal comparison purposes. The percent identity between the two sequences is a function of the number of identical positions shared by the sequences (i.e., percent identity=number of identical positions/total number of positions (e.g., overlapping positions)×100). In one embodiment, the two sequences are the same length. The percent identity between two sequences can be determined using techniques similar to those described below, with or without allowing gaps. In calculating percent identity, typically exact matches are counted.

The determination of percent identity between two sequences can be accomplished using a mathematical algorithm. A preferred, nonlimiting example of a mathematical algorithm utilized for the comparison of two sequences is the algorithm of Karlin and Altschul (1990) Proc. Natl. Acad. Sci. USA 87:2264, modified as in Karlin and Altschul (1993) Proc. Natl. Acad. Sci. USA 90:5873-5877. Such an algorithm is incorporated into the NBLAST and XBLAST programs of Altschul et al. (1990) J. Mol. Biol. 215:403. BLAST nucleotide searches can be performed with the NBLAST program, score=100, wordlength=12, to obtain nucleotide sequences homologous to the polynucleotide molecules of the invention. BLAST protein searches can be performed with the XBLAST program, score=50, wordlength=3, to obtain amino acid sequences homologous to protein molecules of the invention. To obtain gapped alignments for comparison purposes, Gapped BLAST can be utilized as described in Altschul et al. (1997) Nucleic Acids Res. 25:3389. Alternatively, PSI-Blast can be used to perform an iterated search that detects distant relationships between molecules. See Altschul et al. (1997) supra. When utilizing BLAST, Gapped BLAST, and PSI-Blast programs, the default parameters of the respective programs (e.g., XBLAST and NBLAST; available on the world-wide web at ncbi.nlm.nih.gov). Another preferred, non-limiting example of a mathematical algorithm utilized for the comparison of sequences is the algorithm of Myers and Miller (1988) CABIOS 4:11-17. Such an algorithm is incorporated into the ALIGN program (version 2.0), which is part of the GCG sequence alignment software package. When utilizing the ALIGN program for comparing amino acid sequences, a PAM120 weight residue table, a gap length penalty of 12, and a gap penalty of 4 can be used. Alignment may also be performed manually by inspection.

Unless otherwise stated, sequence identity/similarity values provided herein refer to the value obtained using the full-length sequences of the invention and using multiple alignment by mean of the algorithm Clustal W (Nucleic Acid Research, 22(22):4673-4680, 1994) using the program AlignX included in the software package Vector NTI Suite Version 7 (InforMax, Inc., Bethesda, MD, USA) using the default parameters; or any equivalent program thereof. By “equivalent program” is intended any sequence comparison program that, for any two sequences in question, generates an alignment having identical nucleotide or amino acid residue matches and an identical percent sequence identity when compared to the corresponding alignment generated by CLUSTALW (Version 1.83) using default parameters (available at the European Bioinformatics Institute website on the world-wide web at: ebi.ac.uk/Tools/clustalw/index.html).

The use of the term “polynucleotide” is not intended to limit the present invention to polynucleotides comprising DNA. Those of ordinary skill in the art will recognize that polynucleotides, can comprise ribonucleotides and combinations of ribonucleotides and deoxyribonucleotides. Such deoxyribonucleotides and ribonucleotides include both naturally occurring molecules and synthetic analogues. The polynucleotides of the invention also encompass all forms of sequences including, but not limited to, single-stranded forms, double-stranded forms, hairpins, stem-and-loop structures, and the like.

The polynucleotide constructs comprising NLR protein coding regions can be provided in expression cassettes for expression in the plant or other organism or in a host cell of interest. The cassette will include 5′ and 3′ regulatory sequences operably linked to the protein coding region. “Operably linked” is intended to mean a functional linkage between two or more elements. For example, an operable linkage between a polynucleotide or gene of interest and a regulatory sequence (i.e., a promoter) is functional link that allows for expression of the polynucleotide of interest. Operably linked elements may be contiguous or non-contiguous. When used to refer to the joining of two protein coding regions, by operably linked is intended that the coding regions are in the same reading frame. The cassette may additionally contain at least one additional gene to be cotransformed into the organism. Alternatively, the additional gene(s) can be provided on multiple expression cassettes. Such an expression cassette is provided with a plurality of restriction sites and/or recombination sites for insertion of the protein coding region to be under the transcriptional regulation of the regulatory regions. The expression cassette may additionally contain selectable marker genes.

The expression cassette will include in the 5′-3′ direction of transcription, a transcriptional and translational initiation region (i.e., a promoter), an NLR protein coding region of the invention, and a transcriptional and translational termination region (i.e., termination region) functional in plants or other organism or non-human host cell. The regulatory regions (i.e., promoters, transcriptional regulatory regions, and translational termination regions) and/or the NLR protein coding region or of the invention may be native/analogous to the host cell or to each other. Alternatively, the NLR gene, the regulatory regions and/or NLR protein coding region of the invention may be heterologous to the host cell or to each other.

As used herein, “heterologous” in reference to a nucleic acid molecule or nucleotide sequence that is present in a species of interest is a nucleic acid molecule or nucleotide sequence that originates from a different species than the species of interest and that is not introduced by introgression or other method that involves sexual reproduction, or, if from the same species, the nucleic acid molecule or nucleotide sequence that is present in a species of interest is modified from its native form in composition and/or genomic locus by deliberate human intervention. For example, a promoter operably linked to a heterologous polynucleotide is from a species different from the species from which the polynucleotide was derived, or, if from the same/analogous species, one or both are substantially modified from their original form and/or genomic locus, or the promoter is not the native promoter for the operably linked polynucleotide. As used herein, a chimeric gene or chimeric polynucleotide construct comprises a coding sequence operably linked to a transcription initiation region that is heterologous to the coding sequence.

The present invention provides host cells comprising at least of the nucleic acid molecules, expression cassettes, and vectors of the present invention. In preferred embodiments of the invention, a host cell is a plant cell. In other embodiments, a host cell is selected from the group consisting of a bacterium, a fungal cell, and an animal cell. In certain embodiments, a host cell is non-human animal cell. However, in some other embodiments, the host cell is an in-vitro cultured human cell.

The termination region may be native with the transcriptional initiation region, may be native with the operably linked NLR protein coding region of interest, may be native with the plant host, or may be derived from another source (i.e., foreign or heterologous to the promoter, the protein of interest, and/or the plant host), or any combination thereof. Convenient termination regions are available from the Ti-plasmid of A. tumefaciens, such as the octopine synthase and nopaline synthase termination regions. See also Guerineau et al. (1991) Mol. Gen. Genet. 262:141-144; Proudfoot (1991) Cell 64:671-674; Sanfacon et al. (1991) Genes Dev. 5:141-149; Mogen et al. (1990) Plant Cell 2:1261-1272; Munroe et al. (1990) Gene 91:151-158; Ballas et al. (1989) Nucleic Acids Res. 17:7891-7903; and Joshi et al. (1987) Nucleic Acids Res. 15:9627-9639.

Where appropriate, the polynucleotides may be optimized for increased expression in the transformed plant. That is, the polynucleotides can be synthesized using plant-preferred codons for improved expression. See, for example, Campbell and Gowri (1990) Plant Physiol. 92:1-11 for a discussion of host-preferred codon usage. Methods are available in the art for synthesizing plant-preferred genes. See, for example, U.S. Pat. Nos. 5,380,831, and 5,436,391, and Murray et al. (1989) Nucleic Acids Res. 17:477-498, herein incorporated by reference.

Additional sequence modifications are known to enhance gene expression in a cellular host. These include elimination of sequences encoding spurious polyadenylation signals, exon-intron splice site signals, transposon-like repeats, and other such well-characterized sequences that may be deleterious to gene expression. The G-C content of the sequence may be adjusted to levels average for a given cellular host, as calculated by reference to known genes expressed in the host cell. When possible, the sequence is modified to avoid predicted hairpin secondary mRNA structures.

Additionally, the polynucleotides can be modified to alter the amino acid sequences of the NLR proteins, for example, to improve translational efficiency, protein stability and/or any other desired property or properties, and/or to reduce any one or more undesirable properties, while improving or at least not reducing significantly the biological activity of the NLR proteins. For example, the polynucleotides can be modified to remove potential allergenic regions in the proteins encoded thereby. See, the AllergenOnline database for a comprehensive list of known and putative allergens (Goodman et al. (2016) Mol. Nutr. Food Res. 60(5):1183-1198; available on the World Wide Web at: allergenonline.org).

The expression cassettes may additionally contain 5′ leader sequences. Such leader sequences can act to enhance translation. Translation leaders are known in the art and include: picornavirus leaders, for example, EMCV leader (Encephalomyocarditis 5′ noncoding region) (Elroy-Stein et al. (1989) Proc. Natl. Acad. Sci. USA 86:6126-6130); potyvirus leaders, for example, TEV leader (Tobacco Etch Virus) (Gallie et al. (1995) Gene 165(2):233-238), MDMV leader (Maize Dwarf Mosaic Virus) (Virology 154:9-20), and human immunoglobulin heavy-chain binding protein (BiP) (Macejak et al. (1991) Nature 353:90-94); untranslated leader from the coat protein mRNA of alfalfa mosaic virus (AMV RNA 4) (Jobling et al. (1987) Nature 325:622-625); tobacco mosaic virus leader (TMV) (Gallie et al. (1989) in Molecular Biology of RNA, ed. Cech (Liss, New York), pp. 237-256); and maize chlorotic mottle virus leader (MCMV) (Lommel et al. (1991) Virology 81:382-385). See also, Della-Cioppa et al. (1987) Plant Physiol. 84:965-968.

In preparing the expression cassette, the various DNA fragments may be manipulated, so as to provide for the DNA sequences in the proper orientation and, as appropriate, in the proper reading frame. Toward this end, adapters (also referred to as “adaptors) or linkers may be employed to join the DNA fragments or other manipulations may be involved to provide for convenient restriction sites, removal of superfluous DNA, removal of restriction sites, or the like. For this purpose, in vitro mutagenesis, primer repair, restriction, annealing, resubstitutions, e.g., transitions and transversions, may be involved.

A number of promoters can be used in the practice of the invention. The promoters can be selected based on the desired outcome. The nucleic acids can be combined with constitutive, tissue-preferred, or other promoters for expression in plants. Such constitutive promoters include, for example, the core CaMV 35S promoter (Odell et al. (1985) Nature 313:810-812); rice actin (McElroy et al. (1990) Plant Cell 2:163-171); ubiquitin (Christensen et al. (1989) Plant Mol. Biol. 12:619-632 and Christensen et al. (1992) Plant Mol. Biol. 18:675-689); pEMU (Last et al. (1991) Theor. Appl. Genet. 81:581-588); MAS (Velten et al. (1984) EMBO J. 3:2723-2730); ALS promoter (U.S. Pat. No. 5,659,026), and the like. Other constitutive promoters include, for example, U.S. Pat. Nos. 5,608,149; 5,608,144; 5,604,121; 5,569,597; 5,466,785; 5,399,680; 5,268,463; 5,608,142; and 6,177,611.

Tissue-preferred promoters can be utilized to target enhanced expression of the R protein coding sequences within a particular plant tissue. Such tissue-preferred promoters include, but are not limited to, leaf-preferred promoters, root-preferred promoters, seed-preferred promoters, and stem-preferred promoters. Tissue-preferred promoters include Yamamoto et al. (1997) Plant J. 12(2):255-265; Kawamata et al. (1997) Plant Cell Physiol. 38(7):792-803; Hansen et al. (1997) Mol. Gen Genet. 254(3):337-343; Russell et al. (1997) Transgenic Res. 6(2):157-168; Rinehart et al. (1996) Plant Physiol. 112(3):1331-1341; Van Camp et al. (1996) Plant Physiol. 112(2):525-535; Canevascini et al. (1996) Plant Physiol. 112(2):513-524; Yamamoto et al. (1994) Plant Cell Physiol. 35(5):773-778; Lam (1994) Results Probl. Cell Differ. 20:181-196; Orozco et al. (1993) Plant Mol Biol. 23(6):1129-1138; Matsuoka et al. (1993) Proc Natl. Acad. Sci. USA 90(20):9586-9590; and Guevara-Garcia et al. (1993) Plant J. 4(3):495-505. Such promoters can be modified, if necessary, for weak expression.

The transgene can be expressed using an inducible promoter, such as, for example, a pathogen-inducible promoter. Such promoters include those from pathogenesis-related proteins (PR proteins), which are induced following infection by a pathogen; e.g., PR proteins, SAR proteins, beta-1,3-glucanase, chitinase, etc. See, for example, Redolfi et al. (1983) Neth. J. Plant Pathol. 89:245-254; Uknes et al. (1992) Plant Cell 4:645-656; and Van Loon (1985) Plant Mol. Virol. 4:111-116. See also WO 99/43819, herein incorporated by reference.

Of interest are promoters that are expressed locally at or near the site of pathogen infection. See, for example, Marineau et al. (1987) Plant Mol. Biol. 9:335-342; Matton et al. (1989) Molecular Plant-Microbe Interactions 2:325-331; Somsisch et al. (1986) Proc. Natl. Acad. Sci. USA 83:2427-2430; Somsisch et al. (1988) Mol. Gen. Genet. 2:93-98; and Yang (1996) Proc. Natl. Acad. Sci. USA 93:14972-14977. See also, Chen et al. (1996) Plant J. 10:955-966; Zhang et al. (1994) Proc. Natl. Acad. Sci. USA 91:2507-2511; Warner et al. (1993) Plant J. 3:191-201; Siebertz et al. (1989) Plant Cell 1:961-968; U.S. Pat. No. 5,750,386 (nematode-inducible); and the references cited therein. Of particular interest is the inducible promoter for the maize PRms gene, whose expression is induced by the pathogen Fusarium moniliforme (see, for example, Cordero et al. (1992) Physiol. Mol. Plant Path. 41:189-200).

Additionally, as pathogens find entry into plants through wounds or insect damage, a wound-inducible promoter may be used in the constructions of the invention. Such wound-inducible promoters include potato proteinase inhibitor (pin II) gene (Ryan (1990) Ann. Rev. Phytopath. 28:425-449; Duan et al. (1996) Nature Biotechnology 14:494-498); wun1 and wun2, U.S. Pat. No. 5,428,148; win1 and win2 (Stanford et al. (1989) Mol. Gen. Genet. 215:200-208); systemin (McGurl et al. (1992) Science 225:1570-1573); WIP1 (Rohmeier et al. (1993) Plant Mol. Biol. 22:783-792; Eckelkamp et al. (1993) FEBS Letters 323:73-76); MPI gene (Corderok et al. (1994) Plant J. 6(2): 141-150); and the like, herein incorporated by reference.

Chemical-regulated promoters can be used to modulate the expression of a gene in a plant through the application of an exogenous chemical regulator. Depending upon the objective, the promoter may be a chemical-inducible promoter, where application of the chemical induces gene expression, or a chemical-repressible promoter, where application of the chemical represses gene expression. Chemical-inducible promoters are known in the art and include, but are not limited to, the maize In2-2 promoter, which is activated by benzenesulfonamide herbicide safeners, the maize GST promoter, which is activated by hydrophobic electrophilic compounds that are used as pre-emergent herbicides, and the tobacco PR-la promoter, which is activated by salicylic acid. Other chemical-regulated promoters of interest include steroid-responsive promoters (see, for example, the glucocorticoid-inducible promoter in Schena et al. (1991) Proc. Natl. Acad. Sci. USA 88:10421-10425 and McNellis et al. (1998) Plant J. 14(2):247-257) and tetracycline-inducible and tetracycline-repressible promoters (see, for example, Gatz et al. (1991) Mol. Gen. Genet. 227:229-237, and U.S. Pat. Nos. 5,814,618 and 5,789,156), herein incorporated by reference.

The expression cassette can also comprise a selectable marker gene for the selection of transformed cells. Selectable marker genes are utilized for the selection of transformed cells or tissues. Marker genes include genes encoding antibiotic resistance, such as those encoding neomycin phosphotransferase II (NEO) and hygromycin phosphotransferase (HPT), as well as genes conferring resistance to herbicidal compounds, such as glufosinate ammonium, bromoxynil, imidazolinones, and 2,4-dichlorophenoxyacetate (2,4-D). Additional selectable markers include phenotypic markers such as β-galactosidase and fluorescent proteins such as green fluorescent protein (GFP) (Su et al. (2004) Biotechnol Bioeng 85:610-9 and Fetter et al. (2004) Plant Cell 16:215-28), cyan florescent protein (CYP) (Bolte et al. (2004) J. Cell Science 117:943-54 and Kato et al. (2002) Plant Physiol 129:913-42), and yellow florescent protein (PhiYFP™ from Evrogen, see, Bolte et al. (2004) J. Cell Science 117:943-54). For additional selectable markers, see generally, Yarranton (1992) Curr. Opin. Biotech. 3:506-511; Christopherson et al. (1992) Proc. Natl. Acad. Sci. USA 89:6314-6318; Yao et al. (1992) Cell 71:63-72; Reznikoff (1992) Mol. Microbiol. 6:2419-2422; Barkley et al. (1980) in The Operon, pp. 177-220; Hu et al. (1987) Cell 48:555-566; Brown et al. (1987) Cell 49:603-612; Figge et al. (1988) Cell 52:713-722; Deuschle et al. (1989) Proc. Natl. Acad. Sci. USA 86:5400-5404; Fuerst et al. (1989) Proc. Natl. Acad. Sci. USA 86:2549-2553; Deuschle et al. (1990) Science 248:480-483; Gossen (1993) Ph.D. Thesis, University of Heidelberg; Reines et al. (1993) Proc. Natl. Acad. Sci. USA 90:1917-1921; Labow et al. (1990) Mol. Cell. Biol. 10:3343-3356; Zambretti et al. (1992) Proc. Natl. Acad. Sci. USA 89:3952-3956; Baim et al. (1991) Proc. Natl. Acad. Sci. USA 88:5072-5076; Wyborski et al. (1991) Nucleic Acids Res. 19:4647-4653; Hillenand-Wissman (1989) Topics Mol. Struc. Biol. 10:143-162; Degenkolb et al. (1991) Antimicrob. Agents Chemother. 35:1591-1595; Kleinschmidt et al. (1988) Biochemistry 27:1094-1104; Bonin (1993) Ph.D. Thesis, University of Heidelberg; Gossen et al. (1992) Proc. Natl. Acad. Sci. USA 89:5547-5551; Oliva et al. (1992) Antimicrob. Agents Chemother. 36:913-919; Hlavka et al. (1985) Handbook of Experimental Pharmacology, Vol. 78 (Springer-Verlag, Berlin); Gill et al. (1988) Nature 334:721-724. Such disclosures are herein incorporated by reference.

The above list of selectable marker genes is not intended to be limiting. Any selectable marker gene can be used in the present invention.

Numerous plant transformation vectors and methods for transforming plants are available. See, for example, An, G. et al. (1986) Plant Pysiol., 81:301-305; Fry, J., et al. (1987) Plant Cell Rep. 6:321-325; Block, M. (1988) Theor. Appl Genet. 76:767-774; Hinchee, et al. (1990) Stadler. Genet. Symp. 203212.203-212; Cousins, et al. (1991) Aust. J. Plant Physiol. 18:481-494; Chee, P. P. and Slightom, J. L. (1992) Gene. 118:255-260; Christou, et al. (1992) Trends. Biotechnol. 10:239-246; D'Halluin, et al. (1992) Bio/Technol. 10:309-314; Dhir, et al. (1992) Plant Physiol. 99:81-88; Casas et al. (1993) Proc. Nat. Acad Sci. USA 90:11212-11216; Christou, P. (1993) In Vitro Cell. Dev. Biol.—Plant; 29P: 119-124; Davies, et al. (1993) Plant Cell Rep. 12:180-183; Dong, J. A. and Mchughen, A. (1993) Plant Sci. 91:139-148; Franklin, C. I. and Trieu, T. N. (1993) Plant. Physiol. 102: 167; Golovkin, et al. (1993) Plant Sci. 90:41-52; Guo Chin Sci. Bull. 38:2072-2078; Asano, et al. (1994) Plant Cell Rep. 13; Ayeres N. M. and Park, W. D. (1994) Crit. Rev. Plant. Sci. 13:219-239; Barcelo, et al. (1994) Plant. J. 5:583-592; Becker, et al. (1994) Plant. J. 5:299-307; Borkowska et al. (1994) Acta. Physiol Plant. 16:225-230; Christou, P. (1994) Agro. Food. Ind. Hi Tech. 5: 17-27; Eapen et al. (1994) Plant Cell Rep. 13:582-586; Hartman, et al. (1994) Bio-Technology 12: 919923; Ritala, et al. (1994) Plant. Mol. Biol. 24:317-325; and Wan, Y. C. and Lemaux, P. G. (1994) Plant Physiol. 104:3748.

Plant transformation vectors that find use in the present invention include, for example, T-DNA vectors or plasmids, which are suitable for use in Agrobacterium-mediated transformation methods that are disclosed elsewhere herein or otherwise known in the art.

The methods of the invention involve introducing a polynucleotide construct into a plant. By “introducing” is intended presenting to the plant the polynucleotide construct in such a manner that the construct gains access to the interior of a cell of the plant. The methods of the invention do not depend on a particular method for introducing a polynucleotide construct to a plant, only that the polynucleotide construct gains access to the interior of at least one cell of the plant. Methods for introducing polynucleotide constructs into plants are known in the art including, but not limited to, stable transformation methods, transient transformation methods, and virus-mediated methods.

By “stable transformation” is intended that the polynucleotide construct introduced into a plant integrates into the genome of the plant and is capable of being inherited by progeny thereof. By “transient transformation” is intended that a polynucleotide construct introduced into a plant does not integrate into the genome of the plant.

For the transformation of plants and plant cells, the nucleotide sequences of the invention are inserted using standard techniques into any vector known in the art that is suitable for expression of the nucleotide sequences in a plant or plant cell. The selection of the vector depends on the preferred transformation technique and the target plant species to be transformed.

Methodologies for constructing plant expression cassettes and introducing foreign nucleic acids into plants are generally known in the art and have been previously described. For example, foreign DNA can be introduced into plants, using tumor-inducing (Ti) plasmid vectors. Other methods utilized for foreign DNA delivery involve the use of PEG mediated protoplast transformation, electroporation, microinjection whiskers, and biolistics or microprojectile bombardment for direct DNA uptake. Such methods are known in the art. (U.S. Pat. No. 5,405,765 to Vasil et al.; Bilang et al. (1991) Gene 100: 247-250; Scheid et al., (1991) Mol. Gen. Genet., 228: 104-112; Guerche et al., (1987) Plant Science 52: 111-116; Neuhause et al., (1987) Theor. Appl Genet. 75: 30-36; Klein et al., (1987) Nature 327: 70-73; Howell et al., (1980) Science 208:1265; Horsch et al., (1985) Science 227: 1229-1231; DeBlock et al., (1989) Plant Physiology 91: 694-701; Methods for Plant Molecular Biology (Weissbach and Weissbach, eds.) Academic Press, Inc. (1988) and Methods in Plant Molecular Biology (Schuler and Zielinski, eds.) Academic Press, Inc. (1989). The method of transformation depends upon the plant cell to be transformed, stability of vectors used, expression level of gene products and other parameters.

Other suitable methods of introducing nucleotide sequences into plant cells and subsequent insertion into the plant genome include microinjection as Crossway et al. (1986) Biotechniques 4:320-334, electroporation as described by Riggs et al. (1986) Proc. Natl. Acad. Sci. USA 83:5602-5606, Agrobacterium-mediated transformation as described by Townsend et al., U.S. Pat. No. 5,563,055, Zhao et al., U.S. Pat. No. 5,981,840, direct gene transfer as described by Paszkowski et al. (1984) EMBO J. 3:2717-2722, and ballistic particle acceleration as described in, for example, Sanford et al., U.S. Pat. No. 4,945,050; Tomes et al., U.S. Pat. No. 5,879,918; Tomes et al., U.S. Pat. No. 5,886,244; Bidney et al., U.S. Pat. No. 5,932,782; Tomes et al. (1995) “Direct DNA Transfer into Intact Plant Cells via Microprojectile Bombardment,” in Plant Cell, Tissue, and Organ Culture: Fundamental Methods, ed. Gamborg and Phillips (Springer-Verlag, Berlin); McCabe et al. (1988) Biotechnology 6:923-926); and Lec1 transformation (WO 00/28058). Also see, Weissinger et al. (1988) Ann. Rev. Genet. 22:421-477; Sanford et al. (1987) Particulate Science and Technology 5:27-37 (onion); Christou et al. (1988) Plant Physiol. 87:671-674 (soybean); McCabe et al. (1988) Bio/Technology 6:923-926 (soybean); Finer and McMullen (1991) In Vitro Cell Dev. Biol. 27P: 175-182 (soybean); Singh et al. (1998) Theor. Appl. Genet. 96:319-324 (soybean); Datta et al. (1990) Biotechnology 8:736-740 (rice); Klein et al. (1988) Proc. Natl. Acad. Sci. USA 85:4305-4309 (maize); Klein et al. (1988) Biotechnology 6:559-563 (maize); Tomes, U.S. Pat. No. 5,240,855; Buising et al., U.S. Pat. Nos. 5,322,783 and 5,324,646; Tomes et al. (1995) “Direct DNA Transfer into Intact Plant Cells via Microprojectile Bombardment,” in Plant Cell, Tissue, and Organ Culture: Fundamental Methods, ed. Gamborg (Springer-Verlag, Berlin) (maize); Klein et al. (1988) Plant Physiol. 91:440-444 (maize); Fromm et al. (1990) Biotechnology 8:833-839 (maize); Hooykaas-Van Slogteren et al. (1984) Nature (London) 311:763-764; Bowen et al., U.S. Pat. No. 5,736,369 (cereals); Bytebier et al. (1987) Proc. Natl. Acad. Sci. USA 84:5345-5349 (Liliaceae); De Wet et al. (1985) in The Experimental Manipulation of Ovule Tissues, ed. Chapman et al. (Longman, New York), pp. 197-209 (pollen); Kaeppler et al. (1990) Plant Cell Reports 9:415-418 and Kaeppler et al. (1992) Theor. Appl. Genet. 84:560-566 (whisker-mediated transformation); D'Halluin et al. (1992) Plant Cell 4:1495-1505 (electroporation); Li et al. (1993) Plant Cell Reports 12:250-255 and Christou and Ford (1995) Annals of Botany 75:407-413 (rice); Osjoda et al. (1996) Nature Biotechnology 14:745-750 (maize via Agrobacterium tumefaciens); all of which are herein incorporated by reference.

The polynucleotides of the invention may be introduced into plants by contacting plants with a virus or viral nucleic acids. Generally, such methods involve incorporating a polynucleotide construct of the invention within a viral DNA or RNA molecule. Further, it is recognized that promoters of the invention also encompass promoters utilized for transcription by viral RNA polymerases. Methods for introducing polynucleotide constructs into plants and expressing a protein encoded therein, involving viral DNA or RNA molecules, are known in the art. See, for example, U.S. Pat. Nos. 5,889,191, 5,889,190, 5,866,785, 5,589,367 and 5,316,931; herein incorporated by reference.

If desired, the modified viruses or modified viral nucleic acids can be prepared in formulations. Such formulations are prepared in a known manner (see e.g. for review U.S. Pat. No. 3,060,084, EP-A 707 445 (for liquid concentrates), Browning, “Agglomeration”, Chemical Engineering, Dec. 4, 1967, 147-48, Perry's Chemical Engineer's Handbook, 4th Ed., McGraw-Hill, New York, 1963, pages 8-57 and et seq. WO 91/13546, U.S. Pat. Nos. 4,172,714, 4,144,050, 3,920,442, 5,180,587, 5,232,701, 5,208,030, GB 2,095,558, U.S. Pat. No. 3,299,566, Klingman, Weed Control as a Science, John Wiley and Sons, Inc., New York, 1961, Hance et al. Weed Control Handbook, 8th Ed., Blackwell Scientific Publications, Oxford, 1989 and Mollet, H., Grubemann, A., Formulation technology, Wiley VCH Verlag GmbH, Weinheim (Germany), 2001, 2. D. A. Knowles, Chemistry and Technology of Agrochemical Formulations, Kluwer Academic Publishers, Dordrecht, 1998 (ISBN 0-7514-0443-8), for example by extending the active compound with auxiliaries suitable for the formulation of agrochemicals, such as solvents and/or carriers, if desired emulsifiers, surfactants and dispersants, preservatives, antifoaming agents, anti-freezing agents, for seed treatment formulation also optionally colorants and/or binders and/or gelling agents.

In specific embodiments, the polynucleotide constructs and expression cassettes of the invention can be provided to a plant using a variety of transient transformation methods known in the art. Such methods include, for example, microinjection or particle bombardment. See, for example, Crossway et al. (1986) Mol Gen. Genet. 202:179-185; Nomura et al. (1986) Plant Sci. 44:53-58; Hepler et al. (1994) PNAS Sci. 91: 2176-2180 and Hush et al. (1994) J. Cell Science 107:775-784, all of which are herein incorporated by reference. Alternatively, the polynucleotide can be transiently transformed into the plant using techniques known in the art. Such techniques include viral vector system and Agrobacterium tumefaciens-mediated transient expression as described elsewhere herein.

The cells that have been transformed may be grown into plants in accordance with conventional ways. See, for example, McCormick et al. (1986) Plant Cell Reports 5:81-84. These plants may then be grown, and either pollinated with the same transformed strain or different strains, and the resulting hybrid having constitutive expression of the desired phenotypic characteristic identified. Two or more generations may be grown to ensure that expression of the desired phenotypic characteristic is stably maintained and inherited and then seeds harvested to ensure expression of the desired phenotypic characteristic has been achieved. In this manner, the present invention provides transformed seed (also referred to as “transgenic seed”) having a polynucleotide construct of the invention, for example, an expression cassette of the invention, stably incorporated into their genome.

Such methods known in the art for modifying DNA in the genome of a plant include, for example, mutation breeding and genome editing techniques, such as, for example, methods involving targeted mutagenesis, site-directed integration (SDI), and homologous recombination. Targeted mutagenesis or similar techniques are disclosed in U.S. Pat. Nos. 5,565,350; 5,731,181; 5,756,325; 5,760,012; 5,795,972, 5,871,984, and 8,106,259; all of which are herein incorporated in their entirety by reference. Methods for gene modification or gene replacement comprising homologous recombination can involve inducing single-strand or double-strand breaks in DNA using zinc-finger nucleases (ZFN), TAL (transcription activator-like) effector nucleases (TALEN), Clustered Regularly Interspaced Short Palindromic Repeats/CRISPR-associated nuclease (CRISPR/Cas nuclease), or homing endonucleases that have been engineered endonucleases to make double-strand breaks at specific recognition sequences in the genome of a plant, other organism, or host cell. See, for example, Durai et al., (2005) Nucleic Acids Res. 33:5978-90; Mani et al. (2005) Biochem. Biophys. Res. Comm 335:447-57; U.S. Pat. Nos. 7,163,824, 7,001,768, and 6,453,242; Arnould et al. (2006) J Mol. Biol. 355:443-58; Ashworth et al., (2006) Nature 441:656-9; Doyon et al. (2006) J Am Chem Soc 128:2477-84; Rosen et al., (2006) Nucleic Acids Res. 34:4791-800; and Smith et al., (2006) Nucleic Acids Res. 34:e149; U.S. Pat. App. Pub. No. 2009/0133152; and U.S. Pat. App. Pub. No. 2007/0117128; all of which are herein incorporated in their entirety by reference.

TAL effector nucleases (TALENs) can be used to make double-strand breaks at specific recognition sequences in the genome of a plant for gene modification or gene replacement through homologous recombination. TAL effector nucleases are a class of sequence-specific nucleases that can be used to make double-strand breaks at specific target sequences in the genome of a plant or other organism. TAL effector nucleases are created by fusing a native or engineered transcription activator-like (TAL) effector, or functional part thereof, to the catalytic domain of an endonuclease, such as, for example, FokI. The unique, modular TAL effector DNA binding domain allows for the design of proteins with potentially any given DNA recognition specificity. Thus, the DNA binding domains of the TAL effector nucleases can be engineered to recognize specific DNA target sites and thus, used to make double-strand breaks at desired target sequences. See, WO 2010/079430; Morbitzer et al. (2010) PNAS 10.1073/pnas. 1013133107; Scholze and Boch (2010) Virulence 1:428-432; Christian et al. Genetics (2010) 186:757-761; Li et al. (2010) Nuc. Acids Res. (2010) doi:10.1093/nar/gkq704; and Miller et al. (2011) Nature Biotechnology 29:143-148; all of which are herein incorporated by reference.

The CRISPR/Cas nuclease system can also be used to make single-strand or double-strand breaks at specific recognition sequences in the genome of a plant for gene modification or gene replacement through homologous recombination. The CRISPR/Cas nuclease is an RNA-guided (simple guide RNA, sgRNA in short) DNA endonuclease system performing sequence-specific double-stranded breaks in a DNA segment homologous to the designed RNA. It is possible to design the specificity of the sequence (Cho S. W. et al., Nat. Biotechnol. 31:230-232, 2013; Cong L. et al., Science 339:819-823, 2013; Mali P. et al., Science 339:823-826, 2013; Feng Z. et al., Cell Research: 1-4, 2013).

In addition, a ZFN can be used to make double-strand breaks at specific recognition sequences in the genome of a plant for gene modification or gene replacement through homologous recombination. The Zinc Finger Nuclease (ZFN) is a fusion protein comprising the part of the FokI restriction endonuclease protein responsible for DNA cleavage and a zinc finger protein which recognizes specific, designed genomic sequences and cleaves the double-stranded DNA at those sequences, thereby producing free DNA ends (Urnov F. D. et al., Nat Rev Genet. 11:636-46, 2010; Carroll D., Genetics. 188:773-82, 2011).

Breaking DNA using site specific nucleases, such as, for example, those described herein above, can increase the rate of homologous recombination in the region of the breakage. Thus, coupling of such effectors as described above with nucleases enables the generation of targeted changes in genomes which include additions, deletions and other modifications.

Unless expressly stated or apparent from the context of usage, the methods and compositions of the present invention can be used with any plant species including, for example, monocotyledonous plants (“monocots”), dicotyledonous plants (“dicots”), and conifers. Examples of plant species of interest include, but are not limited to, corn (Zea mays), Brassica sp. (e.g., B. napus, B. rapa, B. juncea), particularly those Brassica species useful as sources of seed oil, alfalfa (Medicago sativa), rice (Oryza sativa), rye (Secale cereale), triticale (x Triticosecale or Triticum x Secale) sorghum (Sorghum bicolor, Sorghum vulgare), teff (Eragrostis tef), millet (e.g., pearl millet (Pennisetum glaucum), proso millet (Panicum miliaceum), foxtail millet (Setaria italica), finger millet (Eleusine coracana)), switchgrass (Panicum virgatum), sunflower (Helianthus annuus), safflower (Carthamus tinctorius), wheat (Triticum aestivum), soybean (Glycine max), tobacco (Nicotiana tabacum), potato (Solanum tuberosum), peanuts (Arachis hypogaea), cotton (Gossypium barbadense, Gossypium hirsutum), strawberry (e.g. Fragaria x ananassa, Fragaria vesca, Fragaria moschata, Fragaria virginiana, Fragaria chiloensis), sweet potato (Ipomoea batatus), yam (Dioscorea spp., D. rotundata, D. cayenensis, D. alata, D. polystachya, D. bulbifera, D. esculenta, D. dumetorum, D. trifida), cassava (Manihot esculenta), coffee (Coffea spp.), coconut (Cocos nucifera), oil palm (e.g. Elaeis guineensis, Elaeis oleifera), pineapple (Ananas comosus), citrus trees (Citrus spp.), cocoa (Theobroma cacao), tea (Camellia sinensis), banana (Musa spp.), avocado (Persea americana), fig (Ficus casica), guava (Psidium guajava), mango (Mangifera indica), olive (Olea europaea), papaya (Carica papaya), cashew (Anacardium occidentale), macadamia (Macadamia integrifolia), almond (Prunus amygdalus), date (Phoenix dactylifera), cultivated forms of Beta vulgaris (sugar beets, garden beets, chard or spinach beet, mangelwurzel or fodder beet), sugarcane (Saccharum spp.), oat (Avena sativa), barley (Hordeum vulgare), cannabis (Cannabis sativa, C. indica, C. ruderalis), poplar (Populus spp.), eucalyptus (Eucalyptus spp.), Arabidopsis thaliana, Arabidopsis rhizogenes, Nicotiana benthamiana, Brachypodium distachyon vegetables, ornamentals, and conifers and other trees. In specific embodiments, plants of the present invention are crop plants (e.g. maize, sorghum, wheat, millet, rice, barley, oats, sugarcane, alfalfa, soybean, peanut, sunflower, cotton, safflower, Brassica spp., lettuce, strawberry, apple, citrus, etc.).

Vegetables include tomatoes (Lycopersicon esculentum), eggplant (also known as “aubergine” or “brinjal”) (Solanum melongena), pepper (Capsicum annuum), lettuce (e.g., Lactuca sativa), green beans (Phaseolus vulgaris), lima beans (Phaseolus limensis), peas (Lathyrus spp.), chickpeas (Cicer arietinum), and members of the genus Cucumis such as cucumber (C. sativus), cantaloupe (C. cantalupensis), and musk melon (C. melo). Ornamentals include azalea (Rhododendron spp.), hydrangea (Macrophylla hydrangea), hibiscus (Hibiscus rosasanensis), roses (Rosa spp.), tulips (Tulipa spp.), daffodils (Narcissus spp.), petunias (Petunia hybrida), carnation (Dianthus caryophyllus), poinsettia (Euphorbia pulcherrima), and chrysanthemum. Fruit trees and related plants include, for example, apples, pears, peaches, plums, oranges, grapefruits, limes, pomelos, palms, and bananas. Nut trees and related plants include, for example, almonds, cashews, walnuts, pistachios, macadamia nuts, filberts, hazelnuts, and pecans.

In specific embodiments, the plants of the present invention are crop plants such as, for example, maize (corn), soybean, wheat, rice, cotton, alfalfa, sunflower, canola (Brassica spp., particularly Brassica napus, Brassica rapa, Brassica juncea), rapeseed (Brassica napus), sorghum, millet, barley, triticale, safflower, peanut, sugarcane, tobacco, potato, tomato, and pepper.

In some preferred embodiments, the methods and compositions of the present invention can be used to enhance the resistance of a crop plants, particularly domesticated wheat plants, to one or more of the following diseases of wheat: wheat stem rust caused by Puccinia graminis f. sp. tritici), wheat stripe rust caused by Puccinia striiformis f. sp. tritici, wheat leaf rust caused by Puccinia triticina and wheat blast caused by Magnaporthe oryzae Triticum. Domesticated wheat plants include, but are not limited to, common wheat or bread wheat (Triticum aestivum), durham wheat (Triticum durum or Triticum turgidum subsp. durum), einkorn wheat (Triticum monococcum), spelt (Triticum spelta), emmer wheat (Triticum turgidum subsp. Dicoccum; Triticum turgidum conv. durum), and khorasan wheat (Triticum turgidum ssp. Turanicum or Triticum turanicum).

The term “plant” is intended to encompass plants at any stage of maturity or development, as well as any cells, tissues or organs (plant parts) taken or derived from any such plant unless otherwise clearly indicated by context. Plant parts include, but are not limited to, fruits, stems, tubers, roots, flowers, ovules, stamens, petals, leaves, hypocotyls, epicotyls, cotyledons, embryos, meristematic regions, callus tissue, anther cultures, gametophytes, sporophytes, pollen, microspores, protoplasts, seeds, and the like. It is recognized that the plant protoplasts of the present invention can be prepared from any one or more of the aforementioned plant parts and at any stage of development and/or maturity.

Likewise, the term “plant cell” is intended to encompass plant cells obtained from or in plants at any stage of maturity or development unless otherwise clearly indicated by context. Plant cells can be from or in plant parts including, but are not limited to, fruits, stems, tubers, roots, flowers, ovules, stamens, leaves, embryos, meristematic regions, callus tissue, anther cultures, gametophytes, sporophytes, pollen, microspores, in vitro-cultured tissues, organs or cells and the like.

Progeny, variants, and mutants of the regenerated plants are also included within the scope of the invention, provided that these parts comprise the introduced polynucleotides. As used herein, “progeny” and “progeny plant” comprise any subsequent generation of a plant whether resulting from sexual reproduction and/or asexual propagation, unless it is expressly stated otherwise or is apparent from the context of usage.

The term “expression” as used herein refers to the biosynthesis of a gene product, including the transcription and/or translation of said gene product. The “expression” or “production” of a protein or polypeptide from a DNA molecule refers to the transcription and translation of the coding sequence to produce the protein or polypeptide, while the “expression” or “production” of a protein or polypeptide from an RNA molecule refers to the translation of the RNA coding sequence to produce the protein or polypeptide. Preferably, for the methods of the present invention unless stated otherwise or apparent from the context of usage, an NLR is an expressed NLR in a plant, a plant organ, or other plant part if mRNA (i.e. transcripts) of the NLR is detected in the plant, the plant organ, or the other plant part.

The use of the terms “DNA” or “RNA” herein is not intended to limit the present invention to polynucleotide molecules comprising DNA or RNA. Those of ordinary skill in the art will recognize that the methods and compositions of the invention encompass nucleic acid molecules, polynucleotides, polynucleotide constructs, expression cassettes, and vectors comprised of deoxyribonucleotides (i.e., DNA), ribonucleotides (i.e., RNA) or combinations of ribonucleotides and deoxyribonucleotides. Such deoxyribonucleotides and ribonucleotides include both naturally occurring molecules and synthetic analogues including, but not limited to, nucleotide analogs or modified backbone residues or linkages, which are synthetic, naturally occurring, and non-naturally occurring, which have similar binding properties as the reference nucleic acid, and which are metabolized in a manner similar to the reference nucleotides. Examples of such analogs include, without limitation, phosphorothioates, phosphoramidates, methyl phosphonates, chiral-methyl phosphonates, 2-O-methyl ribonucleotides, peptide-nucleic acids (PNAs). The polynucleotide molecules of the invention also encompass all forms of polynucleotide molecules including, but not limited to, single-stranded forms, double-stranded forms, hairpins, stem-and-loop structures, and the like. Furthermore, it is understood by those of ordinary skill in the art that the nucleotide sequences disclosed herein also encompasses the complement of that exemplified nucleotide sequence.

The invention is drawn to compositions and methods for producing a plant with enhanced resistance to a plant disease caused by one, two, three, four or more plant pathogens. By “resistance to a plant disease” or “disease resistance” is intended that the plants avoid the disease symptoms that are the outcome of plant-pathogen interactions. That is, one or more pathogens are prevented from causing a plant disease or plant diseases and the associated disease symptoms, or alternatively, the disease symptoms caused by the one or more pathogens is minimized or lessened.

While the methods of method for preparing a library of candidate R genes and methods of identifying R genes have been largely described for R genes against plant pathogens that cause plant disease to plant of the interest, the methods of the present invention are broadly applicable to R genes against any plant pest including, but not limited to, plant pathogens (e.g. fungi, oomycetes, bacteria, viruses, and nematodes) and insects, and acarids that cause damage to plants. Thus, the term “plant pathogen” as used herein encompasses any plant pest unless expressly stated or apparent from the context of usage. Similarly, term “plant disease” or “disease” as used herein encompasses any damage caused to a plant by a plant pest unless expressly stated or apparent from the context of usage.

Plant pathogens include, for example, bacteria, fungi, oomycetes, viruses, nematodes, and the like. Specific pathogens for the major crops include: Soybeans: Phytophthora megasperma fsp. glycinea, Macrophomina phaseolina, Rhizoctonia solani, Sclerotinia sclerotiorum, Fusarium oxysporum, Diaporthe phaseolorum var. sojae (Phomopsis sojae), Diaporthe phaseolorum var. caulivora, Sclerotium rolfsii, Cercospora kikuchii, Cercospora sojina, Peronospora manshurica, Colletotrichum dematium (Colletotrichum truncatum), Corynespora cassiicola, Septoria glycines, Phyllosticta sojicola, Alternaria alternata, Pseudomonas syringae p.v. glycinea, Xanthomonas campestris p.v. phaseoli, Microsphaera diffusa, Fusarium semitectum, Phialophora gregata, Soybean mosaic virus, Glomerella glycines, Tobacco Ring spot virus, Tobacco Streak virus, Phakopsora pachyrhizi, Pythium aphanidermatum, Pythium ultimum, Pythium debaryanum, Tomato spotted wilt virus, Heterodera glycines Fusarium solani; Canola: Albugo candida, Alternaria brassicae, Leptosphaeria maculans, Rhizoctonia solani, Sclerotinia sclerotiorum, Mycosphaerella brassicicola, Pythium ultimum, Peronospora parasitica, Fusarium roseum, Alternaria alternata; Alfalfa: Clavibacter michiganensis subsp. insidiosum, Pythium ultimum, Pythium irregulare, Pythium splendens, Pythium debaryanum, Pythium aphanidermatum, Phytophthora megasperma, Peronospora trifoliorum, Phoma medicaginis var. medicaginis, Cercospora medicaginis, Pseudopeziza medicaginis, Leptotrochila medicaginis, Fusarium oxysporum, Verticillium albo-atrum, Xanthomonas campestris p.v. alfalfae, Aphanomyces euteiches, Stemphylium herbarum, Stemphylium alfalfae, Colletotrichum trifolii, Leptosphaerulina briosiana, Uromyces striatus, Sclerotinia trifoliorum, Stagonospora meliloti, Stemphylium botryosum, Leptotrichia medicaginis; Wheat: Pseudomonas syringae p.v. atrofaciens, Urocystis agropyri, Xanthomonas campestris p.v. translucens, Pseudomonas syringae p.v. syringae, Alternaria alternata, Cladosporium herbarum, Fusarium graminearum, Fusarium avenaceum, Fusarium culmorum, Ustilago tritici, Ascochyta tritici, Cephalosporium gramineum, Collotetrichum graminicola, Erysiphe graminis f.sp. tritici, Puccinia graminis f.sp. tritici, Puccinia graminis f.sp. hordei, Puccinia graminis f.sp. avenae, Puccinia graminis f.sp. secalis, Puccinia recondita f.sp. tritici, Puccinia striiformis, Pyrenophora tritici-repentis, Septoria nodorum, Septoria tritici, Septoria avenae, Pseudocercosporella herpotrichoides, Rhizoctonia solani, Rhizoctonia cerealis, Gaeumannomyces graminis var. tritici, Pythium aphanidermatum, Pythium arrhenomanes, Pythium ultimum, Bipolaris sorokiniana, Barley Yellow Dwarf Virus, Brome Mosaic Virus, Soil Borne Wheat Mosaic Virus, Wheat Streak Mosaic Virus, Wheat Spindle Streak Virus, American Wheat Striate Virus, Claviceps purpurea, Tilletia tritici, Tilletia laevis, Ustilago tritici, Tilletia indica, Rhizoctonia solani, Pythium arrhenomannes, Pythium gramicola, Pythium aphanidermatum, High Plains Virus, European wheat striate virus; Sunflower: Plasmopara halstedii, Sclerotinia sclerotiorum, Aster Yellows, Septoria helianthi, Phomopsis helianthi, Alternaria helianthi, Alternaria zinniae, Botrytis cinerea, Phoma macdonaldii, Macrophomina phaseolina, Erysiphe cichoracearum, Rhizopus oryzae, Rhizopus arrhizus, Rhizopus stolonifer, Puccinia helianthi, Verticillium dahliae, Erwinia carotovorum pv. carotovora, Cephalosporium acremonium, Phytophthora cryptogea, Albugo tragopogonis; Corn: Colletotrichum graminicola, Fusarium moniliforme var. subglutinans, Erwinia stewartii, Gibberella zeae (Fusarium graminearum), Fusarium verticilloides, Stenocarpella maydis (Diplodia maydis), Pythium irregulare, Pythium debaryanum, Pythium graminicola, Pythium splendens, Pythium ultimum, Pythium aphanidermatum, Aspergillus flavus, Bipolaris maydis O, T (Cochliobolus heterostrophus), Helminthosporium carbonum I, II & III (Cochliobolus carbonum), Exserohilum turcicum I, II & III, Helminthosporium pedicellatum, Physoderma maydis, Phyllosticta maydis, Kabatiella maydis, Cercospora sorghi, Ustilago maydis, Puccinia sorghi, Puccinia polysora, Macrophomina phaseolina, Penicillium oxalicum, Nigrospora oryzae, Cladosporium herbarum, Curvularia lunata, Curvularia inaequalis, Curvularia pallescens, Clavibacter michiganense subsp. nebraskense, Trichoderma viride, Maize Dwarf Mosaic Virus A & B, Wheat Streak Mosaic Virus, Maize Chlorotic Dwarf Virus, Claviceps sorghi, Pseudonomas avenae, Erwinia chrysanthemi pv. zea, Erwinia carotovora, Corn stunt spiroplasma, Diplodia macrospora, Sclerophthora macrospora, Peronosclerospora sorghi, Peronosclerospora philippinensis, Peronosclerospora maydis, Peronosclerospora sacchari, Sphacelotheca reiliana, Physopella zeae, Cephalosporium maydis, Cephalosporium acremonium, Maize Chlorotic Mottle Virus, High Plains Virus, Maize Mosaic Virus, Maize Rayado Fino Virus, Maize Streak Virus, Maize Stripe Virus, Maize Rough Dwarf Virus; Sorghum: Exserohilum turcicum, C. sublineolum, Cercospora sorghi, Gloeocercospora sorghi, Ascochyta sorghina, Pseudomonas syringae p.v. syringae, Xanthomonas campestris p.v. holcicola, Pseudomonas andropogonis, Puccinia purpurea, Macrophomina phaseolina, Perconia circinata, Fusarium moniliforme, Alternaria alternata, Bipolaris sorghicola, Helminthosporium sorghicola, Curvularia lunata, Phoma insidiosa, Pseudomonas avenae (Pseudomonas alboprecipitans), Ramulispora sorghi, Ramulispora sorghicola, Phyllachara sacchari, Sporisorium reilianum (Sphacelotheca reiliana), Sphacelotheca cruenta, Sporisorium sorghi, Sugarcane mosaic H, Maize Dwarf Mosaic Virus A & B, Claviceps sorghi, Rhizoctonia solani, Acremonium strictum, Sclerophthona macrospora, Peronosclerospora sorghi, Peronosclerospora philippinensis, Sclerospora graminicola, Fusarium graminearum, Fusarium verticillioides, Fusarium oxysporum, Pythium arrhenomanes, Pythium graminicola, etc.; Tomato: Corynebacterium michiganense pv. michiganense, Pseudomonas syringae pv. tomato, Ralstonia solanacearum, Xanthomonas vesicatoria, Xanthomonas perforans, Alternaria solani, Alternaria porri, Collectotrichum spp., Fulvia fulva Syn. Cladosporium fulvum, Fusarium oxysporum f. lycopersici, Leveillula taurica/Oidiopsis taurica, Phytophthora infestans, other Phytophthora spp., Pseudocercospora fuligena Syn. Cercospora fuligena, Sclerotium rolfsii, Septoria lycopersici, Meloidogyne spp.; Potato: Ralstonia solanacearum, Pseudomonas solanacearum, Erwinia carotovora subsp. Atroseptica Erwinia carotovora subsp. Carotovora, Pectobacterium carotovorum subsp. Atrosepticum, Pseudomonas fluorescens, Clavibacter michiganensis subsp. Sepedonicus, Corynebacterium sepedonicum, Streptomyces scabiei, Colletotrichum coccodes, Alternaria alternate, Mycovellosiella concors, Cercospora solani, Macrophomina phaseolina, Sclerotium bataticola, Choanephora cucurbitarum, Puccinia pittieriana, Aecidium cantensis, Alternaria solani, Fusarium spp., Phoma solanicola f. foveata, Botrytis cinerea, Botryotinia fuckeliana, Phytophthora infestans, Pythium spp., Phoma andigena var. andina, Pleospora herbarum, Stemphylium herbarum, Erysiphe cichoracearum, Spongospora subterranean Rhizoctonia solani, Thanatephorus cucumeris, Rosellinia sp. Dematophora sp., Septoria lycopersici, Helminthosporium solani, Polyscytalum pustulans, Sclerotium rolfsii, Athelia rolfsii, Angiosorus solani, Ulocladium atrum, Verticillium albo-atrum, V. dahlia, Synchytrium endobioticum, Sclerotinia sclerotiorum, candidatus Liberibacter solanacearum; Banana: Fusarium oxysporum f. sp. cubense, Colletotrichum musae, Armillaria mellea, Armillaria tabescens, Pseudomonas solanacearum, Phyllachora musicola, Mycosphaerella fijiensis, Rosellinia bunodes, Pseudonomas spp., Pestalotiopsis leprogena, Cercospora hayi, Pseudomonas solanacearum, Ceratocystis paradoxa, Verticillium theobromae, Trachysphaera fructigena, Cladosporium musae, Junghuhnia vincta, Cordana johnstonii, Cordana musae, Fusarium pallidoroseum, Colletotrichum musae, Verticillium theobromae, Fusarium spp., Acremonium spp., Cylindrocladium spp., Deightoniella torulosa, Nattrassia mangiferae, Dreschslera gigantean, Guignardia musae, Botryosphaeria ribis, Fusarium solani, Nectria haematococca, Fusarium oxysporum, Rhizoctonia spp., Colletotrichum musae, Uredo musae, Uromyces musae, Acrodontium simplex, Curvularia eragrostidis, Drechslera musae-sapientum, Leptosphaeria musarum, Pestalotiopsis disseminate, Ceratocystis paradoxa, Haplobasidion musae, Marasmiellus inoderma, Pseudomonas solanacearum, Radopholus similis, Lasiodiplodia theobromae, Fusarium pallidoroseum, Verticillium theobromae, Pestalotiopsis palmarum, Phaeoseptoria musae, Pyricularia grisea, Fusarium moniliforme, Gibberella fujikuroi, Erwinia carotovora, Erwinia chrysanthemi, Cylindrocarpon musae, Meloidogyne arenaria, Meloidogyne incognita, Meloidogyne javanica, Pratylenchus coffeae, Pratylenchus goodeyi, Pratylenchus brachyurus, Pratylenchus reniformia, Sclerotinia sclerotiorum, Nectria foliicola, Mycosphaerella musicola, Pseudocercospora musae, Limacinula tenuis, Mycosphaerella musae, Helicotylenchus multicinctus, Helicotylenchus dihystera, Nigrospora sphaerica, Trachysphaera frutigena, Ramichloridium musae, Verticillium theobromae, Phytophthora infestans, Phytophthora parasitica, Phytophthora ramorum, Phytophthora ipomoeae, Phytophthora mirabilis, Phytophthora capsici, Phytophthora porri, Phytophthora sojae, Phytophthora palmivora, and Phytophthora phaseoli.

Bacterial pathogens include, but are not limited to, Agrobacterium tumefaciens, candidatus Liberibacter asiaticus, candidatus Liberibacter solanacearum, Clavibacter michiganensis, Clavibacter sepedonicus, Dickeya dadantii, Dickeya solani, Erwinia amylovora, Pectobacterium atrosepticum, Pectobacterium carotovorum, Pseudomonas andropogonis, Pseudomonas avenae, Pseudomonas alboprecipitans, Pseudomonas fluorescens, Pseudomonas savastanoi, Pseudomonas solanacearum, Pseudomonas syringae, Ralstonia solanacearum, Xanthomonas axonopodis, Xanthomonas campestris, Xanthomonas citri, Xanthomonas perforans, Xanthomonas vesicatoria, Xanthomonas oryzae, and Xylella fastidiosa.

Oomycete pathogens include, but are not limited to, Phytophthora infestans, Phytophthora ipomoeae, Phytophthora mirabilis, Phytophthora phaseoli, Phytophthora megasperma fsp. glycinea, Phytophthora megasperma, Phytophthora cryptogea, Peronospora spp. and Pythium spp.

Nematode pathogens include, but are not limited to, Anguina tritici, Aphelenchoides besseyi, Bursaphelenchus xylophilus, Ditylenchus dipsaci, Globodera spp., Globodera pallida, Globodera rostochiensis, Heterodera spp., Heterodera avenae, Heterodera filipjevi, Heterodera glycines, Meloidogyne spp., Meloidogyne graminicola, Meloidogyne hapla, Meloidogyne incógnita, Meloidogyne enterolobii, Merlinius spp., Nacobbus aberrans, Paratylenchus spp., Pratylenchus coffeae, Pratylenchus neglectus, Pratylenchus penetrans, Pratylenchus penetrans, Pratylenchus thornei, Pratylenchus vulnus, Pratylenchus zeae, Radopholus similis, Rotylenchulus reniformis, Tylenchorhynchus spp., and Xiphinema index.

Insect pests include, but are not limited to, insects selected from the orders Coleoptera, Diptera, Hymenoptera, Lepidoptera, Mallophaga, Homoptera, Hemiptera, Orthoptera, Dermaptera, Isoptera, Anoplura, Siphonaptera, Thysanoptera, Trichoptera, etc., particularly Coleoptera and Lepidoptera.

Insects of the order Lepidoptera include, but are not limited to, armyworms, cutworms, loopers, and heliothines in the family Noctuidae Agrotis ipsilon Hufnagel (black cutworm); A. orthogonia Morrison (western cutworm); A. segetum Denis & Schiffermüller (turnip moth); A. subterranea Fabricius (granulate cutworm); Alabama argillacea Hübner (cotton leaf worm); Anticarsia gemmatalis Hübner (velvetbean caterpillar); Athetis mindara Barnes and McDunnough (rough skinned cutworm); Earias insulana Boisduval (spiny bollworm); E. vittella Fabricius (spotted bollworm); Egira (Xylomyges) curialis Grote (citrus cutworm); Euxoa messoria Harris (darksided cutworm); Helicoverpa armigera Hübner (American bollworm); H. zea Boddie (corn earworm or cotton bollworm); Heliothis virescens Fabricius (tobacco budworm); Hypena scabra Fabricius (green cloverworm); Hyponeuma taltula Schaus; (Mamestra configurata Walker (bertha armyworm); M. brassicae Linnaeus (cabbage moth); Melanchra picta Harris (zebra caterpillar); Mocis latipes Guenée (small mocis moth); Pseudaletia unipuncta Haworth (armyworm); Pseudoplusia includens Walker (soybean looper); Richia albicosta Smith (Western bean cutworm); Spodoptera frugiperda JE Smith (fall armyworm); S. exigua Hübner (beet armyworm); S. litura Fabricius (tobacco cutworm, cluster caterpillar); Trichoplusia ni Hübner (cabbage looper); borers, casebearers, webworms, coneworms, and skeletonizers from the families Pyralidae and Crambidae such as Achroia grisella Fabricius (lesser wax moth); Amyelois transitella Walker (naval orangeworm); Anagasta kuehniella Zeller (Mediterranean flour moth); Cadra cautella Walker (almond moth); Chilo partellus Swinhoe (spotted stalk borer); C. suppressalis Walker (striped stem/rice borer); C. terrenellus Pagenstecher (sugarcane stemp borer); Corcyra cephalonica Stainton (rice moth); Crambus caliginosellus Clemens (corn root webworm); C. teterrellus Zincken (bluegrass webworm); Cnaphalocrocis medinalis Guenée (rice leaf roller); Desmia funeralis Hübner (grape leaffolder); Diaphania hyalinata Linnaeus (melon worm); D. nitidalis Stoll (pickleworm); Diatraea flavipennella Box; D. grandiosella Dyar (southwestern corn borer), D. saccharalis Fabricius (surgarcane borer); Elasmopalpus lignosellus Zeller (lesser cornstalk borer); Papaipema nebris (stalk borer); Eoreuma loftini Dyar (Mexican rice borer); Ephestia elutella Hübner (tobacco (cacao) moth); Galleria mellonella Linnaeus (greater wax moth); Hedylepta accepta Butler (sugarcane leafroller); Herpetogramma licarsisalis Walker (sod webworm); Homoeosoma electellum Hulst (sunflower moth); Loxostege sticticalis Linnaeus (beet webworm); Maruca testulalis Geyer (bean pod borer); Orthaga thyrisalis Walker (tea tree web moth); Ostrinia nubilalis Hübner (European corn borer); Ostrinia furnacalis (Asian corn borer); Plodia interpunctella Hübner (Indian meal moth); Scirpophaga incertulas Walker (yellow stem borer); Udea rubigalis Guenée (celery leaftier); and leafrollers, budworms, seed worms, and fruit worms in the family Tortricidae Acleris gloverana Walsingham (Western blackheaded budworm); A. variana Fernald (Eastern blackheaded budworm); Hellula phidilealis (cabbage budworm moth); Adoxophyes orana Fischer von Rösslerstamm (summer fruit tortrix moth); Archips spp. including A. argyrospila Walker (fruit tree leaf roller) and A. rosana Linnaeus (European leaf roller); Argyrotaenia spp.; Bonagota salubricola Meyrick (Brazilian apple leafroller); Choristoneura spp.; Cochylis hospes Walsingham (banded sunflower moth); Cydia latiferreana Walsingham (filbertworm); C. pomonella Linnaeus (codling moth); Endopiza viteana Clemens (grape berry moth); Eupoecilia ambiguella Hübner (vine moth); Grapholita molesta Busck (oriental fruit moth); Lobesia botrana Denis & Schiffermüller (European grape vine moth); Platynota flavedana Clemens (variegated leafroller); P. stultana Walsingham (omnivorous leafroller); Spilonota ocellana Denis & Schiffermüller (eyespotted bud moth); and Suleima helianthana Riley (sunflower bud moth).

Selected other agronomic pests in the order Lepidoptera include, but are not limited to, Alsophila pometaria Harris (fall cankerworm); Anarsia lineatella Zeller (peach twig borer); Anisota senatoria J. E. Smith (orange striped oakworm); Antheraea pernyi Guérin-Méneville (Chinese Oak Silkmoth); Bombyx mori Linnaeus (Silkworm); Bucculatrix thurberiella Busck (cotton leaf perforator); Colias eurytheme Boisduval (alfalfa caterpillar); Datana integerrima Grote & Robinson (walnut caterpillar); Dendrolimus sibiricus Tschetwerikov (Siberian silk moth), Ennomos subsignaria Hübner (elm spanworm); Erannis tiliaria Harris (linden looper); Erechthias flavistriata Walsingham (sugarcane bud moth); Euproctis chrysorrhoea Linnaeus (browntail moth); Harrisina americana Guérin-Méneville (grapeleaf skeletonizer); Heliothis subflexa Guenée; Hemileuca oliviae Cockrell (range caterpillar); Hyphantria cunea Drury (fall webworm); Keiferia lycopersicella Walsingham (tomato pinworm); Lambdina fiscellaria fiscellaria Hulst (Eastern hemlock looper); L. fiscellaria lugubrosa Hulst (Western hemlock looper); Leucoma salicis Linnaeus (satin moth); Lymantria dispar Linnaeus (gypsy moth); Malacosoma spp.; Manduca quinquemaculata Haworth (five spotted hawk moth, tomato hornworm); M. sexta Haworth (tomato hornworm, tobacco hornworm); Operophtera brumata Linnaeus (winter moth); Orgyia spp.; Paleacrita vernata Peck (spring cankerworm); Papilio cresphontes Cramer (giant swallowtail, orange dog); Phryganidia californica Packard (California oakworm); Phyllocnistis citrella Stainton (citrus leafminer); Phyllonorycter blancardella Fabricius (spotted tentiform leafminer); Pieris brassicae Linnaeus (large white butterfly); P. rapae Linnaeus (small white butterfly); P. napi Linnaeus (green veined white butterfly); Platyptilia carduidactyla Riley (artichoke plume moth); Plutella xylostella Linnaeus (diamondback moth); Pectinophora gossypiella Saunders (pink bollworm); Pontia protodice Boisduval & Leconte (Southern cabbageworm); Sabulodes aegrotata Guenée (omnivorous looper); Schizura concinna J. E. Smith (red humped caterpillar); Sitotroga cerealella Olivier (Angoumois grain moth); Telchin licus Drury (giant sugarcane borer); Thaumetopoea pityocampa Schiffermüller (pine processionary caterpillar); Tineola bisselliella Hummel (webbing clothesmoth); Tuta absoluta Meyrick (tomato leafminer) and Yponomeuta padella Linnaeus (ermine moth).

Of interest are larvae and adults of the order Coleoptera including weevils from the families Anthribidae, Chrysomelidae, and Curculionidae including, but not limited to: Bruchus pisorum (pea weevil), Callosobruchus maculatus (cowpea weevil), Anthonomus grandis Boheman (boll weevil); Cylindrocopturus adspersus LeConte (sunflower stem weevil); Diaprepes abbreviatus Linnaeus (Diaprepes root weevil); Hypera punctata Fabricius (clover leaf weevil); Lissorhoptrus oryzophilus Kuschel (rice water weevil); Metamasius hemipterus hemipterus Linnaeus (West Indian cane weevil); M. hemipterus sericeus Olivier (silky cane weevil); Sitophilus zeamais (maize weevil); Sitophilus granarius Linnaeus (granary weevil); S. oryzae Linnaeus (rice weevil); Smicronyx fulvus LeConte (red sunflower seed weevil); S. sordidus LeConte (gray sunflower seed weevil); Sphenophorus maidis Chittenden (maize billbug); S. livis Vaurie (sugarcane weevil); Rhabdoscelus obscurus Boisduval (New Guinea sugarcane weevil); flea beetles, cucumber beetles, rootworms, leaf beetles, potato beetles, and leafminers in the family Chrysomelidae including, but not limited to: Cerotoma trifurcata (bean leaf beetle), Chaetocnema ectypa Horn (desert corn flea beetle); C. pulicaria Melsheimer (corn flea beetle); Colaspis brunnea Fabricius (grape colaspis); Diabrotica barberi Smith & Lawrence (northern corn rootworm); D. undecimpunctata howardi Barber (southern corn rootworm); D. virgifera virgifera LeConte (western corn rootworm); Leptinotarsa decemlineata Say (Colorado potato beetle); Oulema melanopus Linnaeus (cereal leaf beetle); Phyllotreta cruciferae Goeze (corn flea beetle); Zygogramma exclamationis Fabricius (sunflower beetle); beetles from the family Coccinellidae including, but not limited to: Epilachna varivestis Mulsant (Mexican bean beetle); chafers and other beetles from the family Scarabaeidae including, but not limited to: Antitrogus parvulus Britton (Childers cane grub); Cyclocephala borealis Arrow (northern masked chafer, white grub); C. immaculata Olivier (southern masked chafer, white grub); Dermolepida albohirtum Waterhouse (Greyback cane beetle); Euetheola humilis rugiceps LeConte (sugarcane beetle); Lepidiota frenchi Blackburn (French's cane grub); Tomarus gibbosus De Geer (carrot beetle); T. subtropicus Blatchley (sugarcane grub); Phyllophaga crinita Burmeister (white grub); P. latifrons LeConte (June beetle); Popillia japonica Newman (Japanese beetle); Rhizotrogus majalis Razoumowsky (European chafer); carpet beetles from the family Dermestidae; wireworms from the family Elateridae, eleodes spp., Melanotus spp. including M. communis Gyllenhal (wireworm); Conoderus spp.; Limonius spp.; Agriotes spp.; Ctenicera spp.; Aeolus spp.; bark beetles from the family Scolytidae; beetles from the family Tenebrionidae; beetles from the family Cerambycidae such as, but not limited to, Migdolus fryanus Westwood (longhorn beetle); and beetles from the Buprestidae family including, but not limited to, Aphanisticus cochinchinae seminulum Obenberger (leaf-mining buprestid beetle).

Adults and immatures of the order Diptera are of interest, including leafminers Agromyza parvicornis Loew (corn blotch leafminer); midges including, but not limited to: Contarinia sorghicola Coquillett (sorghum midge); Mayetiola destructor Say (Hessian fly); Neolasioptera murtfeldtiana Felt, (sunflower seed midge); Sitodiplosis mosellana Géhin (wheat midge); fruit flies (Tephritidae), Bactrocera oleae (olive fruit fly), Ceratitis capitata (Mediterranean fruit fly), Oscinella frit Linnaeus (frit flies); maggots including, but not limited to: Delia spp. including Delia platura Meigen (seedcorn maggot); D. coarctata Fallen (wheat bulb fly); Fannia canicularis Linnaeus, F. femoralis Stein (lesser house flies); Meromyza americana Fitch (wheat stem maggot); Musca domestica Linnaeus (house flies); Stomoxys calcitrans Linnaeus (stable flies)); face flies, horn flies, blow flies, Chrysomya spp.; Phormia spp.; and other muscoid fly pests, horse flies Tabanus spp.; bot flies Gastrophilus spp.; Oestrus spp.; cattle grubs Hypoderma spp.; deer flies Chrysops spp.; Melophagus ovinus Linnaeus (keds); and other Brachycera, mosquitoes Aedes spp.; Anopheles spp.; Culex spp.; black flies Prosimulium spp.; Simulium spp.; biting midges, sand flies, sciarids, and other Nematocera.

Agronomically important members from the order Hemiptera include, but are not limited to: Acrosternum hilare Say (green stink bug); Acyrthisiphon pisum Harris (pea aphid); Adelges spp. (adelgids); Adelphocoris rapidus Say (rapid plant bug); Anasa tristis De Geer (squash bug); Aphis craccivora Koch (cowpea aphid); A. fabae Scopoli (black bean aphid); A. gossypii Glover (cotton aphid, melon aphid); A. maidiradicis Forbes (corn root aphid); A. pomi De Geer (apple aphid); A. spiraecola Patch (spirea aphid); Aulacaspis tegalensis Zehntner (sugarcane scale); Aulacorthum solani Kaltenbach (foxglove aphid); Bemisia argentifolii (silverleaf whitefly); Bemisia tabaci Gennadius (tobacco whitefly, sweetpotato whitefly); B. argentifolii Bellows & Perring (silverleaf whitefly); Blissus leucopterus leucopterus Say (chinch bug); Blostomatidae spp.; Brevicoryne brassicae Linnaeus (cabbage aphid); Cacopsylla pyricola Foerster (pear psylla); Calocoris norvegicus Gmelin (potato capsid bug); Chaetosiphon fragaefolii Cockerell (strawberry aphid); Cimicidae spp.; Coreidae spp.; Corythuca gossypii Fabricius (cotton lace bug); Cyrtopeltis modesta Distant (tomato bug); C. notatus Distant (suckfly); Deois flavopicta Stål (spittlebug); Dialeurodes citri Ashmead (citrus whitefly); Diaphnocoris chlorionis Say (honeylocust plant bug); Diuraphis noxia Kurdjumov/Mordvilko (Russian wheat aphid); Duplachionaspis divergens Green (armored scale); Dysaphis plantaginea Paaserini (rosy apple aphid); Dysdercus suturellus Herrich-Schäffer (cotton stainer); Dysmicoccus boninsis Kuwana (gray sugarcane mealybug); Empoasca fabae Harris (potato leafhopper); Eriosoma lanigerum Hausmann (woolly apple aphid); Erythroneoura spp. (grape leafhoppers); Eumetopina flavipes Muir (Island sugarcane planthopper); Eurygaster spp.; Euschistus servus Say (brown stink bug); E. variolarius Palisot de Beauvois (one-spotted stink bug); Graptostethus spp. (complex of seed bugs); and Hyalopterus pruni Geoffroy (mealy plum aphid); Icerya purchasi Maskell (cottony cushion scale); Labopidicola allii Knight (onion plant bug); Laodelphax striatellus Fallen (smaller brown planthopper); Leptoglossus corculus Say (leaf-footed pine seed bug); Leptodictya tabida Herrich-Schaeffer (sugarcane lace bug); Lipaphis erysimi Kaltenbach (turnip aphid); Lygocoris pabulinus Linnaeus (common green capsid); Lygus lineolaris Palisot de Beauvois (tarnished plant bug); L. hesperus Knight (Western tarnished plant bug); L. pratensis Linnaeus (common meadow bug); L. rugulipennis Poppius (European tarnished plant bug); Macrosiphum euphorbiae Thomas (potato aphid); Macrosteles quadrilineatus Forbes (aster leafhopper); Magicicada septendecim Linnaeus (periodical cicada); Mahanarva fimbriolata Stål (sugarcane spittlebug); M. posticata Stål (little cicada of sugarcane); Melanaphis sacchari Zehntner (sugarcane aphid); Melanaspis glomerata Green (black scale); Metopolophium dirhodum Walker (rose grain aphid); Myzus persicae Sulzer (peach-potato aphid, green peach aphid); Nasonovia ribisnigri Mosley (lettuce aphid); Nephotettix cinticeps Uhler (green leafhopper); N. nigropictus Stål (rice leafhopper); Nezara viridula Linnaeus (southern green stink bug); Nilaparvata lugens Stål (brown planthopper); Nysius ericae Schilling (false chinch bug); Nysius raphanus Howard (false chinch bug); Oebalus pugnax Fabricius (rice stink bug); Oncopeltus fasciatus Dallas (large milkweed bug); Orthops campestris Linnaeus; Pemphigus spp. (root aphids and gall aphids); Peregrinus maidis Ashmead (corn planthopper); Perkinsiella saccharicida Kirkaldy (sugarcane delphacid); Phylloxera devastatrix Pergande (pecan phylloxera); Planococcus citri Risso (citrus mealybug); Plesiocoris rugicollis Fallen (apple capsid); Poecilocapsus lineatus Fabricius (four-lined plant bug); Pseudatomoscelis seriatus Reuter (cotton fleahopper); Pseudococcus spp. (other mealybug complex); Pulvinaria elongata Newstead (cottony grass scale); Pyrilla perpusilla Walker (sugarcane leafhopper); Pyrrhocoridae spp.; Quadraspidiotus perniciosus Comstock (San Jose scale); Reduviidae spp.; Rhopalosiphum maidis Fitch (corn leaf aphid); R. padi Linnaeus (bird cherry-oat aphid); Saccharicoccus sacchari Cockerell (pink sugarcane mealybug); Scaptacoris castanea Perty (brown root stink bug); Schizaphis graminum Rondani (greenbug); Sipha flava Forbes (yellow sugarcane aphid); Sitobion avenae Fabricius (English grain aphid); Sogatella furcifera Horvath (white-backed planthopper); Sogatodes oryzicola Muir (rice delphacid); Spanagonicus albofasciatus Reuter (whitemarked fleahopper); Therioaphis maculata Buckton (spotted alfalfa aphid); Tinidae spp.; Toxoptera aurantii Boyer de Fonscolombe (black citrus aphid); and T. citricida Kirkaldy (brown citrus aphid); Trialeurodes vaporariorum (greenhouse whitefly); Trialeurodes abutiloneus (bandedwinged whitefly) and T. vaporariorum Westwood (greenhouse whitefly); Trioza diospyri Ashmead (persimmon psylla); Typhlocyba pomaria McAtee (white apple leafhopper); Homalodisca vitripennis (glassy winged sharpshooter); Cicadulina mbila (maize leafhopper); Circulifer tenellus (beet leafhopper); Daktulosphaira vitifoliae (grape phylloxera); Coccus pseudomagnoliarum (citricola scale); Coccus hesperidum (soft brown scale); Pulvinaria regalis (horse chestnut scale); Pulvinaria psidii (green shield scale); Aonidiella aurantii (California citrus scale); Aonidiella taxus (Asiatic red scale); Aspidiotus excisus (Cyanotis scale); Aspidiotus nerii (oleander scale); Aulacaspis rosarum (Asiatic rose scale); Aulacaspis tubercularis (white mango scale); Chionaspis lepineyi (oak scurfy scale); Hemiberlesia lataniae (latania scale); Kuwanaspis pseudoleucaspis (bamboo diaspidid scale; Lepidosaphes pini (pine oystershell scale); Lopholeucaspis japonica (Japanese maple scale); Oceanaspidiotus spinosus (spined scale insect); Parlatoria ziziphi (black parlatoria scale); Pseudaonidia duplex (camphor scale); Unaspis yanonensis (arrowhead scale); Phenacoccus solani (Solanum mealybug); Planococcus citri (citrus mealybug); Planococcus (ficus vine mealybug); Pseudococcus longispinus (long-tailed mealybug); Pseudococcus affinis (glasshouse mealybug); Diaphorina citri (Asian citrus psyllid); and Bactericera cockerelli (potato psyllid).

Insects of the order Thysanoptera include, but are not limited to, Thrips tabaci (potato thrips) and Frankliniella occidentalis (western flower thrips).

Other insects of interest include, but are not limited to, grasshopper species (e.g. Schistocerca americana and crickets (e.g. Teleogryllus taiwanemma, Teleogryllus emma).

Acarids are arachnids (Class Arachnida) that are members of the subclass Arci which comprise mites and ticks. While acarids are not true insects, acarids are often grouped together with insect pests of plants because both acarids and insects are members of the phylum Arthropoda. As used herein, the term “insects” encompasses both true insects and acarids unless stated otherwise or apparent from the context of usage. Acarids of interest include, but are not limited to: Aceria tosichella Keifer (wheat curl mite); Panonychus ulmi Koch (European red mite); Petrobia latens Müller (brown wheat mite); Steneotarsonemus bancrofti Michael (sugarcane stalk mite) spider mites and red mites in the family Tetranychidae, Oligonychus grypus Baker & Pritchard, O. indicus Hirst (sugarcane leaf mite), O. pratensis Banks (Banks grass mite), O. stickneyi McGregor (sugarcane spider mite); Tetranychus urticae Koch (two spotted spider mite); T. mcdanieli McGregor (McDaniel mite); T. cinnabarinus Boisduval (carmine spider mite); T. turkestani Ugarov & Nikolski (strawberry spider mite), flat mites in the family Tenuipalpidae, Brevipalpus lewisi McGregor (citrus flat mite); rust and bud mites in the family Eriophyidae.

Additional embodiments of the methods and compositions of the present invention are described elsewhere herein.

The following examples are offered by way of illustration and not by way of limitation.

### EXAMPLES

### Example 1: Preparation of a Library of Candidate NLR Genes

Based on the observations of the present inventors that all characterized NLR resistance genes to foliar pathogens in both monocots and dicots are expressed in unchallenged leaf tissue, the present inventors endeavored to prepare a library of candidate NLR resistance genes from unchallenged leaf tissues from a collection of grass species for the purpose of identifying R genes against plant pathogens of interest. Published examples of such NLR genes that are expressed in unchallenged leaf tissues include, for example, CcRpp1, Pm3b, Rpg1, Rpg5 and Sr33 (Bruggeman et al. (2002) PNAS 99(14) 9328-9333, doi: 10.1073/pnas. 142284999; Bruggeman et al. (2008) PNAS 105(39):14970-5, doi: 10.1073/pnas.0807270105; Kawashima et al., 2016, Nature Biotechnol. 2016 34(6):661-665; U.S. Pat. No. 10,842,097; Yahiaoui et al. (2004) Plant J. 37:528-538, doi: 10.1046/j.1365-313X.2003.01977.x). Unpublished examples of such NLR genes include candidate NLR genes for rps2, Rps6, Rps8, Yrr1, Yrr2, and Yrr3. Interestingly, the present inventors discovered that the average number of NLRs expressed in a leaf transcriptome is relatively low (approx. 125), and the NLR genes identified so far that encode efficacious NLRs are expressed in unchallenged leaf tissue. Furthermore, the top 25% of NLRs expressed in leaf tissue appears to be highly enriched for efficacious NLRs. We have combined this key insight with the ability to rapidly transform genes into wheat and have generated a stably transformed library constructed from over 1,000 diverse grass NLRs in wheat.

Previous work has established molecular and evolutionary signatures of NLRs that contribute to plant immunity such as gene family and rapid evolution (Yang et al., 2013, PNAS 110:18572-18577). Such features Previous work has established molecular and evolutionary signatures of NLRs that contribute to plant immunity such as gene family and rapid evolution (Yang et al., 2013, PNAS 110:18572-18577). Such features of interest include, but are not limited to:
the presence of intraspecific variation in the amino acid sequence encoded by an NLR;
the absence of intraspecific variation in the amino acid sequence encoded by an NLR;
the presence of interspecific variation in the amino acid sequence encoded by an NLR;
the absence of interspecific variation in the amino acid sequence encoded by an NLR; and
substantial intraspecific allelic variation in the amino acid sequence encoded by an NLR.

As used in the Examples herein, a “construct” is specific NLR that has been cloned either into an entry vector or a destination vector: a T1 family is seed derived from a single T0 plant, and a T2 family is a seed derived from a single T1 plant.

### Materials and Methods

### Plants Materials and Growth Conditions

Seeds of the following grass species were used for the preparation of libraries of candidate NLR genes: Achnatherum hymenoides, Aegilops bicornis, Aegilops longissima, Aegilops searsii, Aegilops sharonensis, Agropyron cristatum, Avena abyssinica, Brachypodium distachyon, Briza media, Cynosurus cristatus, Echinaria capitata, Holcus lanatus, Hordeum vulgare, Koeleria macrantha, Lolium perenne, Melica ciliata, Phalaris coerulescens, and Poa trivialis.

Seeds were germinated on damp filter paper on petri dishes and placed at 4° C. for 6-7 days to break seed dormancy. Germinated seeds were transferred into a 1:1 mixture of peat & sand: cereal mix. Seedlings were grown in a clean controlled environment chamber under 16 hrs light at 20° C./8 hrs dark at 16° C. dark. The controlled environment chamber used is a clean germination room free of pests and disease. First and second leaves were harvested per plant per species dependent on leaf size and used for RNA isolation at 12 to 35 days post germination dependent on species.

### RNA Isolation

Total RNA was extracted from leaves using a Trizol-phenol based protocol according to manufacturer's protocol (Sigma-Aldrich; T9424).

### RNAseq

Barcoded Illumina TruSeq RNA HT libraries were constructed and pooled with four samples per lane on a single HiSeq 2500 lane run in Rapid Run mode. Sequencing was performed using 150 bp paired-end reads. Paired end reads were assessed for quality using FastQC and trimmed before assembly using Trimmomatic (v0.36) with parameters set at ILLUMINACLIP: 2:30:10, LEADING: 5, TRAILING: 5, SLIDINGWINDOW: 4:15, and MINLEN: 36. These parameters were used to remove all reads with adapter sequence, ambiguous bases, or a substantial reduction in read quality. De novo transcriptome assemblies were generated using Trinity with default parameters (version 2013 Nov. 10). Kallisto (v0.43.1) was used to estimate expression levels for all transcripts using default parameters and 100 bootstraps.

### Identification of Highly Expressed NLRs

TransDecoder (v4.1.0) LongOrfs was used to predict all open reading frames in de novo assembled transcriptomes. InterProScan (v5.27-66.0) was used to annotate domains using Coils and the Pfam, Superfamily, and ProSite databases. Any protein that contained both a nucleotide binding domain and a leucine-rich repeat domain was advanced in the analysis. A custom script developed from FAT-CAT was used to classify nucleotide binding domains based on a phylogenetic tree developed from rice, Brachypodium distachyon, and barley nucleotide binding domains derived from NLRs. NLR encoding genes were advanced based on the following requirements: the transcript must contain either a complete or 5′ partial open reading frame; the gene must be among the top 25% expressed NLRs; and the gene does not belong to NLR families known to require an additional NLR (Bailey et al. (2018) Genome Biol. 19:23, doi.org/10.1186/s13059-018-1392-6).

Among the candidate NLRs, redundancy was removed using CD-HIT (v4.7) requiring 100% identity (-c 1.0). PCR primers were developed using Gateway adapters attB1 (SEQ ID NO: 27) and attB2 (SEQ ID NO: 28) fused to first 20 nucleotides of the start or end of the coding sequence, respectively.

### Example 2: Testing of Candidate NLR Genes in Transgenic Plants

### NLR Identification and Molecular Cloning

Sequencing, de novo RNAseq assembly, NLR identification, and PCR primer development was completed for plant 81 accessions from 18 grass species. Of the 81 accessions sequenced, 69 resistant accessions were progressed to molecular cloning including species in the genera Achnatherum, Aegilops, Agropyron, Avena, Brachypodium, Briza, Cynosurus, Echinaria, Holcus, Hordeum, Koeleria, Lolium, Melica, Phalaris, and Poa.

The proportion of cloned NLRs is variable according to species, guided by the available diversity of accessions in each species and the prevalence of resistance to target pathogens. PCR primers were developed for a total of 1,909 NLRs. In total, 1,019 NLRs have been cloned into Gateway pDONR entry vector. This set includes the control genes Mla3 (wheat blast), Mla7 (wheat stripe rust), Mla8 (wheat stripe rust), and Rps6 (wheat stripe rust). Additional controls have been identified and synthesised: Sr33 (wheat stem rust), Sr50 (wheat stem rust), Sr35 (wheat stem rust), Pm3 (wheat powdery mildew), Lr21 (wheat leaf rust), and Yr10 (wheat stripe rust).

The NLRs in the entry clones were transferred to a destination vector pDEST2BL, which is a binary vector, by LR reaction of the Gateway® system. The resultant transformation vectors were introduced into Agrobacterium tumefaciens strain EHA105 by electroporation. The Agrobacterium strains carrying the transformation vectors were used to transform a wheat variety Fielder according to the published method (Ishida et al. (2015) Methods Mol. Biol. 1223: 189-198) with a modification that an immature embryo was cut into three pieces when transferred to the second selection medium.

### Pathogen Assays

The library of candidate NLR genes was tested against multiple pathogens of wheat, including wheat stem rust (Puccinia graminis f. sp. tritici), wheat stripe rust (Puccinia striiformis f. sp. tritici), wheat leaf rust (Puccinia triticina), wheat blast (Magnaporthe oryzae Triticum) and wheat powdery mildew (Blumeria graminis f. sp. tritici). The experimental design for seedling pathogen assays involved inoculating three seeds from at least four different Tl family per NLR. Families displaying resistant phenotypes were saved for seed and re-phenotyping at the T2 stage, eight seed were grown and phenotyped at the T2 stage.

Status definitions of screened NLRs are as follows: confirmed NLRs have consistent resistant or intermediate phenotypic scores for T2 families or individuals, derived from a resistant T1 family. Candidate NLRs have borderline intermediate phenotypic scores displayed in T2 families and/or insufficient data to make a conclusion. NLRs for rescreening have susceptible phenotypes shown across T2 families, including from previously resistant or intermediate T1 families. These T2 families may represent NLRs conferring intermediate resistance or NLRs which have insufficient expression under the current promoter.

Wheat Stem Rust (Puccinia graminis f. sp. tritici)

Rust inoculations were made according to the standard protocols used at the USDA-ARS Cereal Disease Laboratory and the University of Minnesota (Huang et al. (2018) Plant Dis. 102(6): 1124-1135, doi: 10.1094/PDIS-06-17-0880-RE). On the day before inoculation, urediniospores of the rust pathogens were removed from the −80° C. freezer, heat-shocked in a 45° C. water bath for 15 min, and then rehydrated in an 80% relative humidity chamber overnight. After assessing the germination rate (Scott et al. 2014), 10 mg of urediniospores were placed into individual gelatin capsules (size 00) to which 700 ml of the oil carrier was added. The inoculum suspension was applied to 12-day-old plants (second leaf fully expanded) using custom atomizers (Tallgrass Solutions, Inc., Manhattan, KS) pressured by a pump set at 25 to 30 kPa. Approximately 0.15 mg of urediniospores were applied per plant. Immediately after inoculation, the plants were placed in front of a small electric fan for 3 to 5 min to hasten evaporation of the oil carrier from leaf surfaces. Plants were allowed to off-gas for an additional 90 min before placing them inside mist chambers. Inside the mist chambers, ultrasonic humidifiers (Vick's model V5100NSJUV; Proctor & Gamble Co., Cincinnati, OH) were run continuously for 30 min to provide sufficient initial moisture on the plants for the germination of urediniospores. For the next 16 to 20 h, plants were kept in the dark and the humidifiers run for 2 min every 15 min to maintain moisture on the plants. For experiments with the stem rust pathogen, light (400 W high pressure sodium lamps emitting 300 mmol photon s-1m-2) was provided for 2 to 4 h after the dark period. Then, the chamber doors were opened halfway to allow the leaf surfaces to dry completely before returning the plants to the greenhouse under the same conditions described above (Huang et al. (2018) Plant Dis. 102(6):1124-1135, doi: 10.1094/PDIS-06-17-0880-RE).

All rust phenotyping experiments were conducted in a completely randomized design and repeated at least once over time. Accessions exhibiting variable reactions across experiments were repeated in an additional experiment, if sufficient seeds were available. Stem rust ITs on the accessions were scored 12 days after inoculation using a 0 to 4 scale (Roelfs and Martens (1998) Phytopathol. 78:526-533; Stakman et al. (1962) “Identification of Physiological Races of Puccinia graminis var. tritici,” U.S. Department of Agricultural Publications E617. USDA, Washington, D C, 1962).

The NLR Dk_04_40 displayed a clear resistant response in the T1, with all individuals from two different T1 families displaying a 0 or; on the Stakman scale.

TABLE 1
Summary of confirmed NLRs following screening of T1 material with stem rust.

| Construct | Species | Accession | Domain Structure | TPM¹ | Status |
|---|---|---|---|---|---|
| Dk_04_40 | Aegilops longissima | 8735 | CC-NB-LRR | 1.0 | Confirmed |

¹Transcripts per million.

Wheat Stripe Rust (Puccinia striiformis f. sp. tritici)

Wheat plants are grown at 18/11 C with a 16 hour day length. For inoculation, wheat plants are inoculated at the first leaf stage with a spore and talc mix 1:16 ratio using a rotary inoculator. Plants are phenotyped 10 days post inoculation using the McNeal phenotypic scale (Roelfs et al., 1992). Resistant individuals were classified by a McNeal score 4 or lower. Intermediate individuals by a McNeal score of 5 to 7 or include either reduced sporulation on the leaf that was clearly differentiable to susceptible controls or sectors of resistance on a leaf (mesothetic response). For ease of phenotyping, some rounds of T2 screening were phenotyped with an overall score of resistant (R), intermediate (I), or susceptible (S) to denote the above McNeal scores.

Confirmed NLRs are derived from 6 accessions from 3 species, with native expression ranging from 0.66 to 5.24 transcripts per million (tpm). Confirmed NLRs are: Dk_01_03, Dk_01_04, Dk_01_06, Dk_01_31, Dk_01_33, Dk_01_34, Dk_01_92, Dk_02_27, Dk_02_28, Dk_02_49, Dk_03_76.

TABLE 2
Screening of T2 families derived from resistant T1 material with wheat stripe
rust (Puccinia striiformis f. sp. tritici) isolate 16/035.

| | | Primary Screen (T1) | | Secondary Screen (T2) | | | |
|---|---|---|---|---|---|---|---|
| NLR | T1 family | 14 dpi | 18 dpi | Score | Mix | Max | Status |
| Dk_01_03 | Dk_01_03_05 | S | 8 | I | I | S | Confirmed |
| Dk_01_03 | Dk_01_03_05 | R | 8 | S | S | S | Confirmed |
| Dk_01_03 | Dk_01_03_05 | R | 7 | S | S | S | Confirmed |
| Dk_01_03 | Dk_01_03_05 | R | 8 | I | I | S | Confirmed |
| Dk_01_03 | Dk_01_03_06 | R | 6 | I | I | S | Confirmed |
| Dk_01_03 | Dk_01_03_06 | R | 7 | I | I | S | Confirmed |
| Dk_01_03 | Dk_01_03_06 | R | 6 | I | 3 | 8 | Confirmed |
| Dk_01_04 | Dk_01_04_02 | Seg | 8 | I | I | I | Confirmed |
| Dk_01_04 | Dk_01_04_02 | Seg | 4 | S | S | S | Confirmed |
| Dk_01_04 | Dk_01_04_02 | Seg | 5 | R | 2 | 6 | Confirmed |
| Dk_01_06 | Dk_01_06_03 | 2Int; 1R | 8 | R | 2 | 7 | Confirmed |
| Dk_01_06 | Dk_01_06_03 | 2Int; 1R | 5 | R | 0 | 6 | Confirmed |
| Dk_01_06 | Dk_01_06_05 | 2Int; 1S | 8 | S | 8 | 9 | Confirmed |
| Dk_01_06 | Dk_01_06_05 | 2Int; 1S | 6 | S | S | S | Confirmed |
| Dk_01_31 | Dk_01_31_04 | 2Int; 1R | 8 | R | 1 | 9 | Confirmed |
| Dk_01_31 | Dk_01_31_04 | 2Int; 1R | 7 | S | S | S | Confirmed |
| Dk_01_33 | Dk_01_33_06 | R | 8 | S | S | S | Confirmed |
| Dk_01_33 | Dk_01_33_06 | R | 8 | S | S | S | Confirmed |
| Dk_01_33 | Dk_01_33_06 | R | 8 | R | 1 | 6 | Confirmed |
| Dk_01_33 | Dk_01_33_06 | R | 6 | R | 0.5 | 6 | Confirmed |
| Dk_01_34 | Dk_01_34_03 | 2Int; 1R | 8 | S | 5 | 6 | Confirmed |
| Dk_01_34 | Dk_01_34_03 | 2Int; 1R | 5 | I | 4 | 6 | Confirmed |
| Dk_01_92 | Dk_01_92_02 | 1R; 1Int; 1S | 8 | R | 0.5 | 4 | Confirmed |
| Dk_01_92 | Dk_01_92_02 | 1R; 1Int; 1S | 3 | R | 0.5 | 4 | Confirmed |
| Dk_02_27 | Dk_02_27_01 | 2Int; 1R | 8 | I | I | S | Confirmed |
| Dk_02_27 | Dk_02_27_01 | 2Int; 1R | 7 | R | R | S | Confirmed |
| Dk_02_27 | Dk_02_27_01 | 2R; 1Int | 8 | R | R | S | Confirmed |
| Dk_02_27 | Dk_02_27_01 | 2R; 1Int | 6 | I | I | S | Confirmed |
| Dk_02_27 | Dk_02_27_01 | 2R; 1Int | 5 | I | 4 | 7 | Confirmed |
| Dk_02_28 | Dk_02_28_05 | 2S; 1R | 8 | I | 4 | 7 | Confirmed |
| Dk_02_28 | Dk_02_28_05 | 2S; 1R | 4 | R | 3 | 6 | Confirmed |
| Dk_02_49 | Dk_02_49_04 | Int | 6 | S | S | S | Confirmed |
| Dk_02_49 | Dk_02_49_04 | Int | 8 | S | S | S | Confirmed |
| Dk_02_49 | Dk_02_49_04 | R | 5 | R | 1 | 8 | Confirmed |
| Dk_02_49 | Dk_02_49_04 | R | 4 | R | 1 | 6 | Confirmed |
| Dk_02_49 | Dk_02_49_04 | R | 4 | R | 1 | 7 | Confirmed |
| Dk_02_49 | Dk_02_49_04 | Int | 7 | I | I | S | Confirmed |
| Dk_02_49 | Dk_02_49_04 | Int | 6 | I | I | S | Confirmed |
| Dk_03_76 | Dk_03_76_06 | 2S; 1R | 8 | S | S | S | Confirmed |
| Dk_03_76 | Dk_03_76_06 | 2S; 1R | (3) | R | 2 | 4 | Confirmed |

TABLE 3
Summary of confirmed NLRs following screening at the T2 with wheat stripe rust.

| Construct | Species | Accession | Domain Structure | tpm | Status |
|---|---|---|---|---|---|
| Dk_01_03 | Aegilops sharonensis | 546 | NB-LRR | 3.28 | Confirmed |
| Dk_01_04 | Aegilops sharonensis | 546 | NB-LRR | 2.21 | Confirmed |
| Dk_01_06 | Aegilops sharonensis | 546 | CC-NB-LRR-CC | 3.03 | Confirmed |
| Dk_01_31 | Aegilops sharonensis | 575 | NB-LRR | 1.59 | Confirmed |
| Dk_01_33 | Aegilops sharonensis | 575 | NB-LRR | 2.16 | Confirmed |
| Dk_01_34 | Aegilops sharonensis | 6793 | NB-LRR-CC-LRR | 5.24 | Confirmed |
| Dk_01_92 | Holcus lanatus | PI462334 | NB-LRR | 3.99 | Confirmed |
| Dk_02_27 | Koeleria macrantha | PI440454 | CC-NB-LRR | 4.56 | Confirmed |
| Dk_02_28 | Koeleria macrantha | PI440454 | NB-LRR | 0.66 | Confirmed |
| Dk_02_49 | Koeleria macrantha | W617985 | NB-LRR | 0.79 | Confirmed |
| Dk_03_76 | Koeleria macrantha | W617985 | NB-LRR | 0.85 | Confirmed |

Wheat Leaf Rust (Puccinia triticina)

Wheat plants are grown at 18/11 C with a 16-hour day length. For inoculation, wheat plants are inoculated at the first leaf stage with a spore and talc mix 1:16 ratio using a rotary inoculator. All isolates were stored as urediniospores at either −80° C. in liquid nitrogen or in vacuum tubes at 4° C. Plants were screened with wheat leaf rust (Puccinia triticina) isolate 13/34 and scored on a standard phenotypic scale used for leaf rust where 0 and; indicate an immune or nearly immune phenotype with no uredinia present. Phenotypic scores of 1 to 2 denote resistance; X, Y, and Z denote varying heterogenous responses; and 3 to 4 denote susceptible responses (Roelfs, 1984, “Race specificity and methods of study,” A P. Roelfs and W. R. Bushnell, eds. The Cereal Rusts Vol. I; Origins, Specificity, Structure, and Physiology. Academic Press, Orlando, pp. 131-164.

The NLR Dk_01_19 exhibited a clear resistance response in the T1, indicating that the transgene was functional against wheat leaf rust. All individuals from 4 Tl families displayed a resistance response of controlled cell death with wrinkled tips, showing small uredinia surrounded by necrosis.

TABLE 4
Summary of confirmed NLRs following screening at the T1 with leaf rust.

| | | | Domain | | |
|---|---|---|---|---|---|
| Construct | Species | Accession | Structure | TPM | Status |
| Dk_01_19 | Aegilops sharonensis | 546 | CC-NB-LRR | 15.6474 | Confirmed |

### Example 3: Identification of Known NLR Genes that Confer Resistance to Oomycetes, Necrotrophic Plant Pathogens, Nematodes, Insects, and Viruses in Subpopulations of Highly Expressed NLRs from Dicots

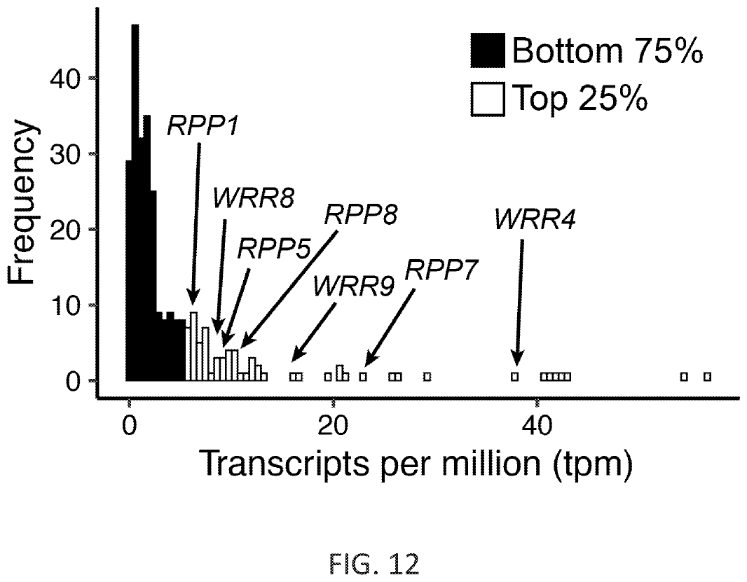

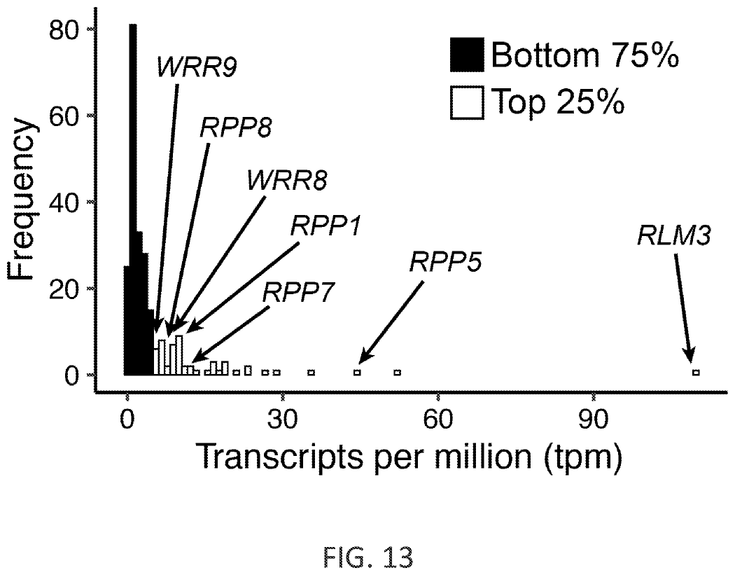

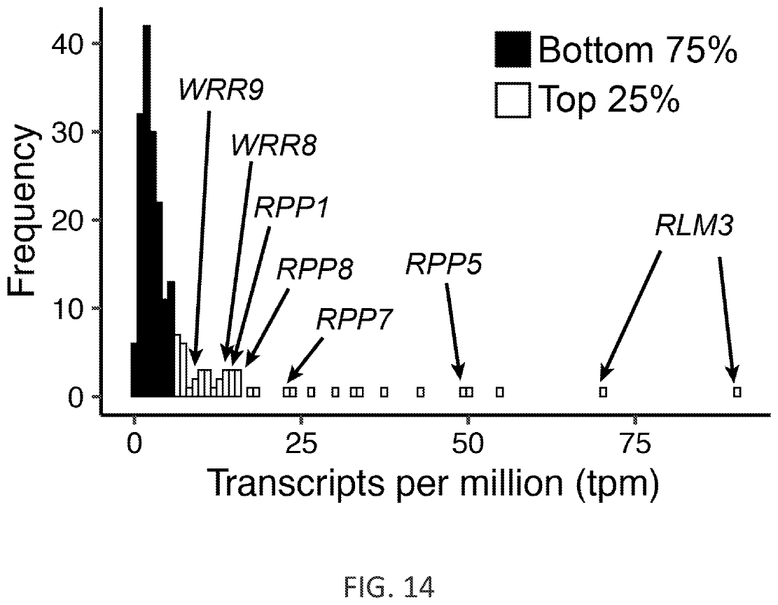

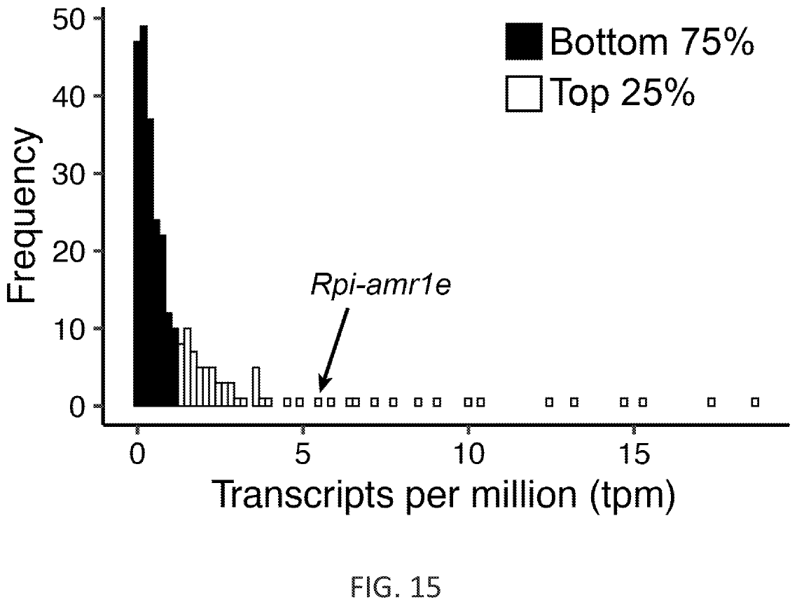

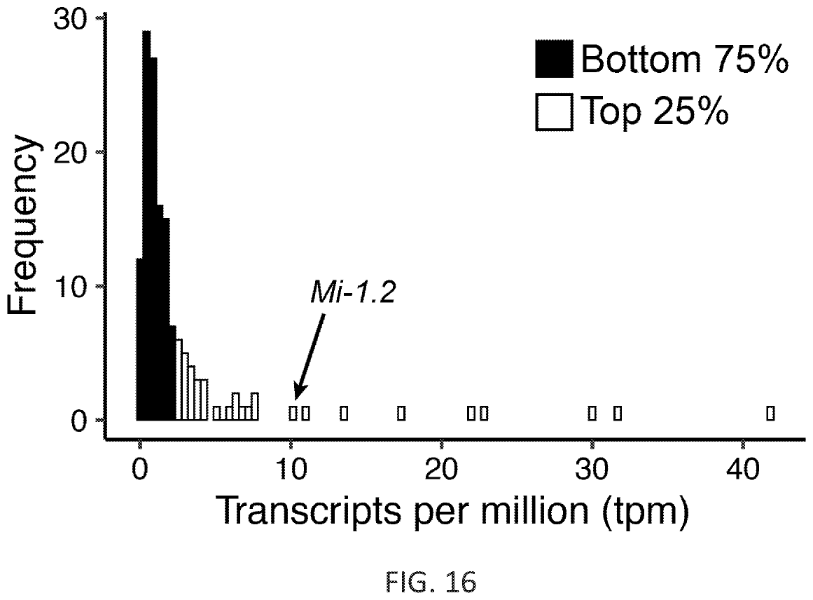

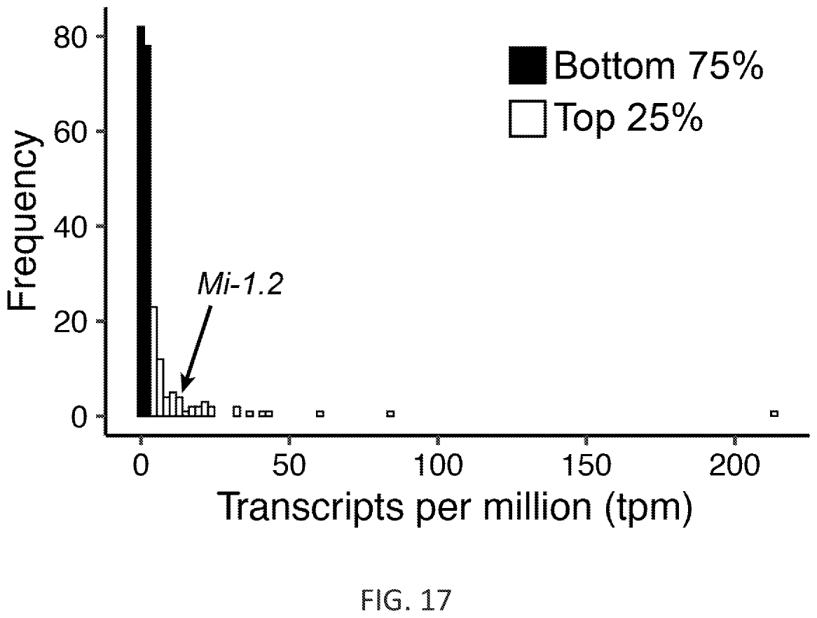

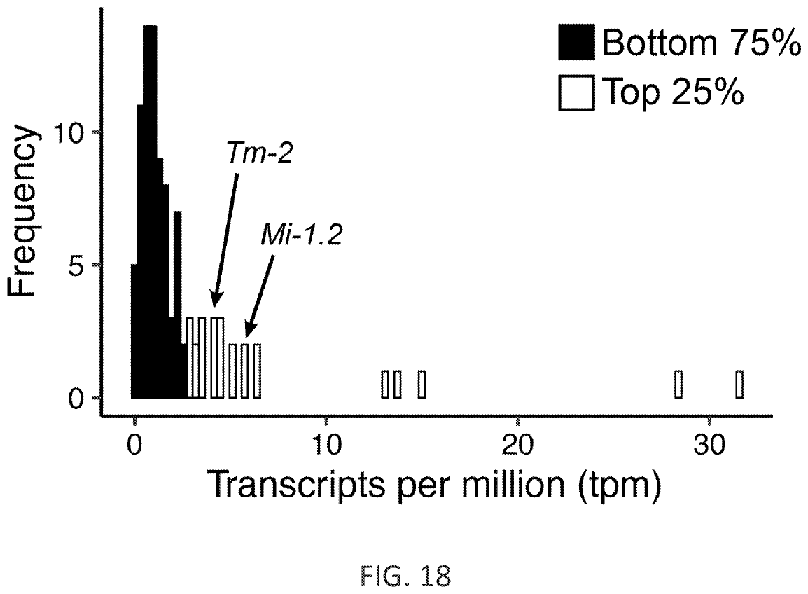

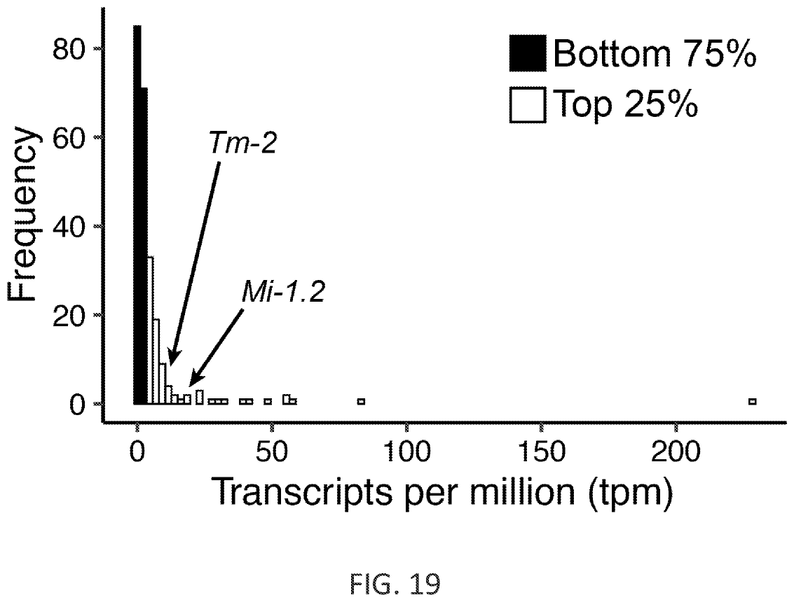

The subgroup of highly expressed NLRs is saturated for functional R genes within the group of NLRs that are expressed in the seedling transcriptome of dicots (FIGS. 12-14). To expand the observations in Arabidopsis thaliana accession Columbia-0 (Col-0), alleles of known resistance genes RPP1, RPP5, RPP7, and RPP8 against late blight (Hyaloperonospora arabidopsidis) and WRR4, WRR8, and WRR9 white rust (Albugo candida) are also present in the top 25% of NLRs expressed in seedlings of further accessions Landsberg erecta (Ler-0), San Feliu (Sf-2), and Wassilewskija (Ws-0). The most highly expressed NLRs in accessions Sf-2 and Ws-0 are alleles of RLM3 which confers resistance to the necrotrophic pathogens grey mould (Botrytis cinerea), dark leaf spot of cabbage (Alternaria brassicicola) and dark spot of crucifers (Alternaria brassicae). To further determine that characterised NLRs identified via association genomics and long-read sequencing could be identified using the above criteria from wild relatives of cultivated plant species, we investigated Rpi-amr1e from Solanum americanum (Witek et al., 2021, Nature Plants 2021 7:198-208). Indeed, Rpi-amr1e was determined to be in the top 25% of highly expressed NLRs (FIG. 15). To confirm high expression of functional NLRs across tissue types, we investigated Mi-1.2 from Solanum lycopersicum which confers resistance to root-knot nematodes (Meloidogyne spp.), the potato aphid (Macrosiphum euphorbiae), and the sweet potato whitefly (Bemisia tabaci). Mi-1.2 is present in the top 10% of highly expressed NLRs in the leaf (FIG. 16 and FIG. 18) and in the top 12% of highly expressed NLRs in the root (FIG. 17 and FIG. 19). Furthermore, the Tm-2 resistance gene to tobamoviruses including Tomato Mosaic Virus and Tobacco Mosaic Virus is present in the top 17% of expressed NLRs in the leaf and in the top 10% of expressed NLRs in the root tissue of the S. lycopersicum cultivar VENT Cherry (FIG. 18 and FIG. 19). These results demonstrate that the methods of the present invention for preparing a library of candidate resistance (R) genes can be employed to produce libraries of candidate R genes that are highly enriched for efficacious R genes, particularly NLRs, against a wide variety of plant pests such as, for example, fungi, bacteria, oomycetes, nematodes, viruses, insects and mites and such libraries can be prepared from not only leaves but also other plant organs or plant parts including, for example, roots.

### Example 4: Testing of Candidate NLR Genes in Transgenic Plants

Wheat Stem Rust (Puccinia graminis f. sp. tritici)

Rust inoculations were made according to the standard protocols used at the USDA-ARS Cereal Disease Laboratory and the University of Minnesota (Huang et al. (2018) Plant Dis. 102(6): 1124-1135, doi: 10.1094/PDIS-06-17-0880-RE). On the day before inoculation, urediniospores of the rust pathogens were removed from the −80° C. freezer, heat-shocked in a 45° C. water bath for 15 min, and then rehydrated in an 80% relative humidity chamber overnight. After assessing the germination rate (Scott et al. 2014), 10 mg of urediniospores were placed into individual gelatin capsules (size 00) to which 700 ml of the oil carrier was added. The inoculum suspension was applied to 12-day-old plants (second leaf fully expanded) using custom atomizers (Tallgrass Solutions, Inc., Manhattan, KS) pressured by a pump set at 25 to 30 kPa. Approximately 0.15 mg of urediniospores were applied per plant. Immediately after inoculation, the plants were placed in front of a small electric fan for 3 to 5 min to hasten evaporation of the oil carrier from leaf surfaces. Plants were allowed to off-gas for an additional 90 min before placing them inside mist chambers. Inside the mist chambers, ultrasonic humidifiers (Vick's model V5100NSJUV; Proctor & Gamble Co., Cincinnati, OH) were run continuously for 30 min to provide sufficient initial moisture on the plants for the germination of urediniospores. For the next 16 to 20 h, plants were kept in the dark and the humidifiers run for 2 min every 15 min to maintain moisture on the plants. For experiments with the stem rust pathogen, light (400 W high pressure sodium lamps emitting 300 mmol photon s-1m-2) was provided for 2 to 4 h after the dark period. Then, the chamber doors were opened halfway to allow the leaf surfaces to dry completely before returning the plants to the greenhouse under the same conditions described above (Huang et al. (2018) Plant Dis. 102(6):1124-1135, doi: 10.1094/PDIS-06-17-0880-RE).

All rust phenotyping experiments were conducted in a completely randomized design. Accessions exhibiting variable reactions across experiments were repeated in an additional experiment, if sufficient seeds were available. Stem rust ITs on the accessions were scored 12 days after inoculation using a 0 to 4 scale (Roelfs and Martens (1998) Phytopathol. 78:526-533; Stakman et al. (1962) “Identification of Physiological Races of Puccinia graminis var. tritici,” U.S. Department of Agricultural Publications E617. USDA, Washington, D C, 1962). ITs are summarised as phenotypes designated as resistant (R), susceptible (S), or segregating (seg) where resistance is segregating within the T1 family. Phenotypes of segregating families indicated as the phenotypes of individual plants. Plants were phenotyped with race QTHJC and resistant constructs further phenotyped with race TTKSK. T1 families not inoculated with TTKSK indicated with ‘-’.

Confirmed NLRs are derived from 14 accessions from 8 species. Confirmed NLRs are: Dk_01_21, Dk_01_48, Dk_03_15, Dk_03_49, Dk_03_68, Dk_04_40, Dk_04_67, Dk_04_71, Dk_04_91, Dk_05_75, Dk_05_92, Dk_06_02, Dk_06_03, Dk_06_10, Dk_06_36, Dk_06_52, Dk_08_16, Dk_08_79, Dk_09_55.

TABLE 5
Screening of T1 families with Wheat stem rust (Puccinia
graminis f. sp. tritici) isolate QTHJC and TTKSK.

| | | Pgt race QTHJC | | Pgt race TTKSK | | T1 Family |
|---|---|---|---|---|---|---|
| Construct | T1 Family | IT | Score | IT | Score | Status |
| Dk_01_21 | Dk_01_21_01 | 2 plants = 2/ | Seg | 3 | S | Segregating |
| | | 2 plants = 3 | | | | |
| Dk_01_21 | Dk_01_21_02 | 1 plants = 2/ | Seg | 1pl = 1, | Seg | Segregating |
| | | 2 plants = 3 | | 1pl = 3 | | |
| Dk_01_48 | Dk_01_48_02 | 0; | R | 0; 1− | S | Confirmed |
| Dk_01_48 | Dk_01_48_02 | 0; 12 | R | 0; 1− | S | Confirmed |
| Dk_01_48 | Dk_01_48_03 | 2 plants = 2/ | Seg | — | — | Segregating |
| | | 1 plant = 3 | | | | |
| Dk_01_48 | Dk_01_48_04 | 2 plants = 2/ | Seg | — | — | Segregating |
| | | 1 plant = 3 | | | | |
| Dk_01_48 | Dk_01_48_06 | 0; 11− | R | 1pl = 3, | Seg | Confirmed |
| | | | | 1pl = 11+ | | |
| Dk_03_15 | Dk_03_15_05 | 2 | R | 3 | S | Confirmed |
| Dk_03_49 | Dk_03_49_04 | 2 | R | 3 | S | Confirmed |
| Dk_03_68 | Dk_03_68_03 | 2 plants = 12/ | Seg | 3 | S | Segregating |
| | | 2 plants = 3 | | | | |
| Dk_04_40 | Dk_04_40_02 | 00; | R | 00; | R | Confirmed |
| Dk_04_40 | Dk_04_40_03 | 00; | R | 00; | R | Confirmed |
| Dk_04_40 | Dk_04_40_03 | 00; | R | 11− | R | Confirmed |
| Dk_04_40 | Dk_04_40_04 | 3 | S | 1 | R | Confirmed |
| Dk_04_67 | Dk_04_67_01 | 2 | R | 11+ | R | Confirmed |
| Dk_04_67 | Dk_04_67_02 | 2 | R | 11+ | R | Confirmed |
| Dk_04_67 | Dk_04_67_02 | 2 | R | 22− | R | Confirmed |
| Dk_04_71 | Dk_04_71_03 | 2 plants = 3/ | Seg | 3 | S | Segregating |
| | | 2 plants = 2 | | | | |
| Dk_04_91 | Dk_04_91_04 | 2 | R | 32 | S | Confirmed |
| Dk_05_75 | Dk_05_75_01 | 0 | R | — | — | Confirmed |
| Dk_05_92 | Dk_05_92_01 | 0 | R | — | — | Confirmed |
| Dk_06_02 | Dk_06_02_05 | 2 | R | 3 | S | Confirmed |
| Dk_06_03 | Dk_06_03_02 | 22+ | R | 3 | S | Confirmed |
| Dk_06_10 | Dk_06_10_04 | 2 | R | 0; 11− | R | Confirmed |
| Dk_06_10 | Dk_06_10_06 | 22+ | R | 3 | S | Confirmed |
| Dk_06_36 | Dk_06_36_03 | 11+ | R | 11− | R | Confirmed |
| Dk_06_52 | Dk_06_52_01 | 11+ | R | 2 | R | Confirmed |
| Dk_06_52 | Dk_06_52_01 | 11+ | R | — | — | Confirmed |
| Dk_06_52 | Dk_06_52_01 | 1 plants = 3/ | Seg | 3 | S | Segregating |
| | | 2 plants = 11+ | | | | |
| Dk_06_52 | Dk_06_52_04 | 1 | R | 11+ | R | Confirmed |
| Dk_06_52 | Dk_06_52_04 | 11+ | R | 11− | R | Confirmed |
| Dk_06_52 | Dk_06_52_04 | 1 | R | 32 | S | Confirmed |
| Dk_06_52 | Dk_06_52_05 | 1 | R | 0; 11− | R | Confirmed |
| Dk_06_52 | Dk_06_52_06 | 1 | R | 3 | S | Confirmed |
| Dk_06_52 | Dk_06_52_06 | 3 plants = 3/ | Seg | 3 | S | Segregating |
| | | 1 plants = 11+ | | | | |
| Dk_08_16 | Dk_08_16_02 | 2 plants = 3/ | Seg | — | — | Segregating |
| | | 1 plant = 0 | | | | |
| Dk_08_79 | Dk_08_79_01 | 1 plant = 3/ | Seg | — | — | Segregating |
| | | 2 plants = 0; 21 | | | | |
| Dk_08_79 | Dk_08_79_01 | 0; 12 | R | — | — | Confirmed |
| Dk_08_79 | Dk_08_79_01 | 1 plant = 32, | Seg | — | — | Segregating |
| | | 2 plants = 0; 21 | | | | |
| Dk_09_55 | Dk_09_55_01 | 11+ | R | — | — | Confirmed |
| Dk_09_55 | Dk_09_55_01 | 11+ | R | — | — | Confirmed |

TABLE 6
Summary of confirmed NLRs following screening at the T1 with stem rust.

| | | | Domain | | |
|---|---|---|---|---|---|
| Construct | Species | Accession | Structure | TPM | Status |
| Dk_01_21 | Aegilops | 575 | NB-LRR | 4.92885 | Confirmed |
| | sharonensis | | | | |
| Dk_01_48 | Aegilops | 546 | CC-NB-LRR | 1.99831 | Confirmed |
| | sharonensis | | | | |
| Dk_03_15 | Cynosurus cristatus | PI251810 | NB-LRR | 3.56616 | Confirmed |
| Dk_03_49 | Holcus lanatus | PI659841 | NB-LRR | 1.99917 | Confirmed |
| Dk_03_68 | Aegilops | 575 | NB-LRR | 2.41543 | Confirmed |
| | sharonensis | | | | |
| Dk_04_40 | Aegilops longissima | 8735 | CC-NB-LRR | 1 | Confirmed |
| Dk_04_67 | Aegilops bicornis | 2327 | CC-NB-LRR | 1.3 | Confirmed |
| Dk_04_71 | Aegilops bicornis | 2327 | CC-NB-LRR | 38.1 | Confirmed |
| Dk_04_91 | Aegilops bicornis | 6065 | CC-NB-LRR | 1.6 | Confirmed |
| Dk_05_75 | Aegilops bicornis | 6065 | NB-LRR | 1.8 | Confirmed |
| Dk_05_92 | Aegilops bicornis | 6150 | NB-LRR | 1 | Confirmed |
| Dk_06_02 | Aegilops longissima | 1059 | CC-NB-LRR | 1.8 | Confirmed |
| Dk_06_03 | Aegilops longissima | 1059 | NB-LRR | 3.7 | Confirmed |
| Dk_06_10 | Aegilops bicornis | 6150 | NB-LRR | 1 | Confirmed |
| Dk_06_36 | Aegilops searsii | 6223 | CC-NB-LRR | 1.95888 | Confirmed |
| Dk_06_52 | Aegilops searsii | 6229 | CC-NB-LRR | 1.27708 | Confirmed |
| Dk_08_16 | Aegilops | 2020 | CC-NB-LRR | 27.1316 | Confirmed |
| | sharonensis | | | | |
| Dk_08_79 | Avena abyssinica | PI158202 | CC-NB-LRR- | 5.19358 | Confirmed |
| | | | CC-LRR | | |
| Dk_09_55 | Briza media | PI3212443 | NB-LRR | 2.77708 | Confirmed |

Wheat Stripe Rust (Puccinia striiformis f. sp. tritici)

Wheat plants are grown at 18/11 C with a 16 hour day length. For inoculation, wheat plants are inoculated at the first leaf stage with a spore and talc mix 1:16 ratio using a rotary inoculator. Plants are phenotyped 10 days post inoculation using the McNeal phenotypic scale (Roelfs et al., 1992). Resistant individuals were classified by a McNeal score 4 or lower. Intermediate individuals by a McNeal score of 5 to 7 or include either reduced sporulation on the leaf that was clearly differentiable to susceptible controls or sectors of resistance on a leaf (mesothetic response). For ease of phenotyping, some rounds of T2 screening were phenotyped with an overall score of resistant (R), intermediate (I), or susceptible (S) to denote the above McNeal scores. Each entry denotes an individual plant derived from a T1 family that was scored as a T2 family in the next generation. Some T1 families were scored as pooled phenotypes for the T1 family, indicated as segregating phenotypes. Where individual McNeal scores for each plant were not saved, this is indicated with ‘-’.

Confirmed NLRs are derived from 18 accessions from 9 species. Confirmed NLRs are: Dk_01_35, Dk_01_55, Dk_01_57, Dk_01_59, Dk_01_60, Dk_01_61, Dk_01_62, Dk_01_64, Dk_01_68, Dk_02_02, Dk_02_03, Dk_02_06, Dk_02_07, Dk_02_08, Dk_02_11, Dk_02_13, Dk_02_14, Dk_02_19, Dk_02_20, Dk_02_25, Dk_02_34, Dk_02_35, Dk_02_36, Dk_02_38, Dk_02_39, Dk_02_42, Dk_02_44, Dk_02_46, Dk_03_13, Dk_03_16, Dk_03_19, Dk_03_48, Dk_03_58, Dk_03_60, Dk_04_34, Dk_04_44, Dk_04_85, Dk_04_88, Dk_04_92, Dk_04_95, Dk_04_96, Dk_05_11, Dk_05_14, Dk_05_15, Dk_05_16, Dk_05_24, Dk_05_29, Dk_05_30, Dk_05_33, Dk_05_34, Dk_05_35, Dk_05_38, Dk_05_42, Dk_05_44, Dk_05_47, Dk_05_53, Dk_05_56, Dk_06_01, Dk_06_03, Dk_06_04, Dk_06_05, Dk_06_06, Dk_06_52, Dk_06_53.

TABLE 7
Screening of T2 families derived from resistant T1 material with wheat stripe
rust (Puccinia striiformis f. sp. tritici) isolate 16/035.

| | | Primary Screen (T1) | | | | |
|---|---|---|---|---|---|---|
| | | | McNeal | Secondary Screen (T2) | | |
| Construct | T1 Family | Score | Score | Score | Min | Max |
| Dk_01_35 | Dk_01_35_02 | 2Int; 1R | — | I | 6 | 9 |
| Dk_01_35 | Dk_01_35_02 | 2Int; 1R | — | I | 6 | 9 |
| Dk_01_55 | Dk_01_55_04 | S | — | I | 6 | 9 |
| Dk_01_55 | Dk_01_55_04 | 2R; 1Int | — | S | 7 | 9 |
| Dk_01_55 | Dk_01_55_04 | 2S; 1Int | — | I | 5 | 9 |
| Dk_01_57 | Dk_01_57_02 | 1R; 1Int; 1S | — | I | 5 | 9 |
| Dk_01_57 | Dk_01_57_02 | 1R; 1Int; 1S | — | I | 5 | 9 |
| Dk_01_57 | Dk_01_57_02 | 2R; 1S | — | R | 4 | 9 |
| Dk_01_57 | Dk_01_57_02 | 2R; 1S | — | I | 5 | 9 |
| Dk_01_59 | Dk_01_59_01 | R | — | I | 5 | 9 |
| Dk_01_59 | Dk_01_59_01 | R | — | I | 5 | 9 |
| Dk_01_59 | Dk_01_59_01 | Int | — | I | 5 | 9 |
| Dk_01_59 | Dk_01_59_07 | 2S; 1R | — | S | 8 | 9 |
| Dk_01_59 | Dk_01_59_07 | 2S; 1R | — | I | 6 | 9 |
| Dk_01_59 | Dk_01_59_07 | 2S; 1R | — | I | 6 | 9 |
| Dk_01_60 | Dk_01_60_01 | R | 1 | S | 7 | 8 |
| Dk_01_60 | Dk_01_60_01 | R | 3 | S | 7 | 9 |
| Dk_01_60 | Dk_01_60_01 | R | 4 | R | 4 | 9 |
| Dk_01_60 | Dk_01_60_01 | R | 2 | S | 7 | 9 |
| Dk_01_60 | Dk_01_60_01 | R | 3 | I | 6 | 9 |
| Dk_01_60 | Dk_01_60_02 | 2S; 1R | — | S | 8 | 9 |
| Dk_01_60 | Dk_01_60_02 | 2S; 1R | — | I | 6 | 9 |
| Dk_01_60 | Dk_01_60_02 | 2S; 1R | — | R | 3 | 9 |
| Dk_01_60 | Dk_01_60_02 | 2S; 1R | — | S | 7 | 9 |
| Dk_01_61 | Dk_01_61_01 | R | — | R | 4 | 9 |
| Dk_01_61 | Dk_01_61_01 | R | — | S | 7 | 9 |
| Dk_01_61 | Dk_01_61_01 | R | — | S | 8 | 9 |
| Dk_01_61 | Dk_01_61_01 | 2R; 1S | — | S | 7 | 9 |
| Dk_01_61 | Dk_01_61_01 | 2R; 1S | — | I | 6 | 9 |
| Dk_01_61 | Dk_01_61_01 | 2R; 1S | — | I | 5 | 9 |
| Dk_01_61 | Dk_01_61_05 | 2R; 1Int | — | I | 6 | 9 |
| Dk_01_61 | Dk_01_61_05 | 2R; 1Int | — | I | 6 | 9 |
| Dk_01_61 | Dk_01_61_05 | 2R; 1Int | — | S | 7 | 9 |
| Dk_01_62 | Dk_01_62_01 | 1R; 1Int; 1S | — | I | 6 | 9 |
| Dk_01_62 | Dk_01_62_01 | 1R; 1Int; 1S | — | I | 6 | 9 |
| Dk_01_64 | Dk_01_64_02 | R | 2 | I | 6 | 9 |
| Dk_01_64 | Dk_01_64_02 | R | 3 | S | 7 | 9 |
| Dk_01_64 | Dk_01_64_02 | R | 3 | S | 7 | 9 |
| Dk_01_68 | Dk_01_68_04 | 2R; 1S | — | I | 6 | 9 |
| Dk_01_68 | Dk_01_68_04 | 2R; 1S | — | S | 7 | 9 |
| Dk_01_68 | Dk_01_68_04 | 2R; 1S | — | R | 4 | 9 |
| Dk_02_02 | Dk_02_02_06 | 2Int; 1S | — | S | 7 | 9 |
| Dk_02_02 | Dk_02_02_06 | 2Int; 1R | — | S | 7 | 9 |
| Dk_02_02 | Dk_02_02_06 | 2Int; 1R | — | I | 5 | 9 |
| Dk_02_03 | Dk_02_03_04 | 2Int; 1R | — | I | 6 | 9 |
| Dk_02_03 | Dk_02_03_04 | R | — | I | 6 | 9 |
| Dk_02_03 | Dk_02_03_04 | R | — | I | 6 | 9 |
| Dk_02_06 | Dk_02_06_02 | R | — | I | 6 | 9 |
| Dk_02_06 | Dk_02_06_02 | 2R; 1Int | — | I | 5 | 9 |
| Dk_02_06 | Dk_02_06_02 | 2R; 1Int | — | I | 6 | 9 |
| Dk_02_07 | Dk_02_07_05 | 2R; 1Int | — | I | 5 | 9 |
| Dk_02_07 | Dk_02_07_05 | 1R; 1Int; 1S | — | I | 5 | 9 |
| Dk_02_08 | Dk_02_08_03 | 1R; 1Int; 1S | — | S | 7 | 9 |
| Dk_02_08 | Dk_02_08_03 | 1R; 1Int; 1S | — | S | 7 | 9 |
| Dk_02_08 | Dk_02_08_06 | 1R; 1Int; 1S | — | I | 6 | 9 |
| Dk_02_08 | Dk_02_08_06 | 2S; 1R | — | S | 7 | 9 |
| Dk_02_11 | Dk_02_11_04 | S | — | I | 5 | 9 |
| Dk_02_11 | Dk_02_11_04 | 2S; 1R | — | S | 7 | 9 |
| Dk_02_13 | Dk_02_13_04 | 2S; 1R | — | R | 3 | 9 |
| Dk_02_13 | Dk_02_13_04 | 2S; 1R | — | I | 5 | 9 |
| Dk_02_13 | Dk_02_13_04 | 2S; 1R | — | I | 6 | 9 |
| Dk_02_13 | Dk_02_13_04 | 1R; 1Int; 1S | — | I | 6 | 8 |
| Dk_02_14 | Dk_02_14_03 | 1R; 1Int; 1S | — | I | 5 | 9 |
| Dk_02_14 | Dk_02_14_03 | 1R; 1Int; 1S | — | R | 4 | 9 |
| Dk_02_19 | Dk_02_19_05 | 1R; 1Int; 1S | — | I | 5 | 9 |
| Dk_02_19 | Dk_02_19_05 | 2Int; 1R | — | I | 5 | 9 |
| Dk_02_20 | Dk_02_20_05 | 2Int; 1R | — | I | 5 | 9 |
| Dk_02_20 | Dk_02_20_05 | 1R; 1Int | — | S | 7 | 9 |
| Dk_02_20 | Dk_02_20_05 | 1R; 1Int | — | I | 5 | 9 |
| Dk_02_25 | Dk_02_25_04 | R | 4 | S | 8 | 9 |
| Dk_02_25 | Dk_02_25_04 | R | 4 | I | 6 | 9 |
| Dk_02_25 | Dk_02_25_04 | I | 6 | S | 7 | 9 |
| Dk_02_34 | Dk_02_34_01 | 2S; 1R | — | I | 5 | 9 |
| Dk_02_34 | Dk_02_34_01 | 2R; 1Int | — | I | 5 | 9 |
| Dk_02_34 | Dk_02_34_01 | 2R; 1Int | — | I | 6 | 9 |
| Dk_02_34 | Dk_02_34_02 | 2R; 1Int | — | I | 6 | 9 |
| Dk_02_34 | Dk_02_34_02 | 2Int; 1R | — | I | 6 | 9 |
| Dk_02_34 | Dk_02_34_02 | 2Int; 1R | — | I | 6 | 9 |
| Dk_02_34 | Dk_02_34_02 | 2R; 1Int | — | I | 6 | 9 |
| Dk_02_35 | Dk_02_35_01 | 2R; 1Int | — | S | 8 | 9 |
| Dk_02_35 | Dk_02_35_01 | 1R; 1Int; 1S | — | S | 8 | 9 |
| Dk_02_35 | Dk_02_35_02 | 2Int; 1R | — | S | 8 | 9 |
| Dk_02_35 | Dk_02_35_02 | 2Int; 1R | — | I | 6 | 9 |
| Dk_02_35 | Dk_02_35_03 | 2S; 1Int | — | I | 6 | 9 |
| Dk_02_35 | Dk_02_35_03 | 1R; 1Int; 1S | — | I | 5 | 9 |
| Dk_02_35 | Dk_02_35_04 | 2R; 1Int | — | I | 5 | 9 |
| Dk_02_35 | Dk_02_35_04 | 2R; 1Int | — | I | 6 | 9 |
| Dk_02_36 | Dk_02_36_03 | R | 1.5 | — | — | — |
| Dk_02_36 | Dk_02_36_03 | R | 4 | — | — | — |
| Dk_02_36 | Dk_02_36_03 | I | 6 | S | 7 | 9 |
| Dk_02_38 | Dk_02_38_07 | R | 4 | I | 6 | 9 |
| Dk_02_38 | Dk_02_38_07 | R | 4 | S | 7 | 9 |
| Dk_02_38 | Dk_02_38_07 | R | 4 | S | 7 | 9 |
| Dk_02_39 | Dk_02_39_07 | 2Int; 1R | — | I | 5 | 9 |
| Dk_02_39 | Dk_02_39_07 | 2S; 1Int | — | I | 6 | 9 |
| Dk_02_42 | Dk_02_42_01 | 2R; 1S | — | R | 4 | 9 |
| Dk_02_42 | Dk_02_42_01 | 2R; 1S | — | I | 5 | 9 |
| Dk_02_42 | Dk_02_42_01 | 2R; 1S | — | I | 5 | 9 |
| Dk_02_44 | Dk_02_44_01 | R | 1 | S | 7 | 9 |
| Dk_02_44 | Dk_02_44_01 | R | 4 | S | 7 | 9 |
| Dk_02_44 | Dk_02_44_01 | I | 6 | S | 7 | 9 |
| Dk_02_44 | Dk_02_44_05 | 2R; 1S | — | I | 5 | 9 |
| Dk_02_44 | Dk_02_44_05 | 2S; 1R | — | I | 5 | 9 |
| Dk_02_46 | Dk_02_46_01 | 1R; 1Int; 1S | — | I | 6 | 9 |
| Dk_02_46 | Dk_02_46_01 | 1R; 1Int; 1S | — | S | 7 | 9 |
| Dk_02_46 | Dk_02_46_01 | 1R; 1Int; 1S | — | I | 5 | 9 |
| Dk_03_13 | Dk_03_13_07 | R | 4 | S | 7 | 9 |
| Dk_03_13 | Dk_03_13_07 | R | 4 | S | 7 | 9 |
| Dk_03_13 | Dk_03_13_07 | R | 4 | I | 6 | 9 |
| Dk_03_13 | Dk_03_13_07 | R | 4 | S | 7 | 9 |
| Dk_03_16 | Dk_03_16_02 | R | 4 | S | 7 | 8 |
| Dk_03_16 | Dk_03_16_02 | R | 4 | I | 6 | 9 |
| Dk_03_16 | Dk_03_16_02 | R | 4 | S | 7 | 9 |
| Dk_03_16 | Dk_03_16_03 | 1R; 1Int; 1S | — | I | 5 | 9 |
| Dk_03_16 | Dk_03_16_03 | 1R; 1Int; 1S | — | S | 7 | 9 |
| Dk_03_19 | Dk_03_19_01 | R | 3.5 | I | 6 | 9 |
| Dk_03_19 | Dk_03_19_01 | R | 4 | R | 4 | 8 |
| Dk_03_19 | Dk_03_19_01 | I | 6 | S | 7 | 9 |
| Dk_03_48 | Dk_03_48_03 | 2S; 1Int | — | I | 5 | 9 |
| Dk_03_48 | Dk_03_48_03 | 2Int; 1R | — | I | 5 | 9 |
| Dk_03_48 | Dk_03_48_03 | 2Int; 1R | — | I | 6 | 9 |
| Dk_03_48 | Dk_03_48_04 | 2Int; 1R | — | I | 6 | 9 |
| Dk_03_48 | Dk_03_48_04 | S | 8 | S | 7 | 9 |
| Dk_03_48 | Dk_03_48_04 | S | 8 | S | 7 | 9 |
| Dk_03_58 | Dk_03_58_07 | I | 6 | S | 7 | 9 |
| Dk_03_58 | Dk_03_58_07 | I | 6 | I | 6 | 8 |
| Dk_03_60 | Dk_03_60_03 | I | 6 | — | — | — |
| Dk_03_60 | Dk_03_60_03 | I | 6 | I | 6 | 9 |
| Dk_03_60 | Dk_03_60_03 | I | 6 | S | 7 | 9 |
| Dk_03_60 | Dk_03_60_03 | I | 6 | I | 6 | 8 |
| Dk_03_60 | Dk_03_60_03 | I | 6 | S | 7 | 9 |
| Dk_04_34 | Dk_04_34_04 | I | 5 | S | 8 | 9 |
| Dk_04_34 | Dk_04_34_04 | S | 7 | S | 7 | 9 |
| Dk_04_34 | Dk_04_34_05 | I | 6 | S | 8 | 9 |
| Dk_04_34 | Dk_04_34_05 | S | 8 | I | 6 | 8 |
| Dk_04_44 | Dk_04_44_02 | I | 6 | I | 5 | 9 |
| Dk_04_44 | Dk_04_44_02 | S | 7 | S | 7 | 9 |
| Dk_04_44 | Dk_04_44_04 | I | 5 | I | 6 | 9 |
| Dk_04_44 | Dk_04_44_04 | S | 7 | S | 8 | 9 |
| Dk_04_85 | Dk_04_85_03 | I | 5 | I | 5 | 8 |
| Dk_04_85 | Dk_04_85_03 | S | 9 | I | 6 | 9 |
| Dk_04_88 | Dk_04_88_01 | I | 6 | S | 7 | 9 |
| Dk_04_88 | Dk_04_88_01 | I | 6 | R | 4 | 9 |
| Dk_04_88 | Dk_04_88_01 | S | 8 | I | 6 | 9 |
| Dk_04_88 | Dk_04_88_01 | S | 7 | R | 0 | 9 |
| Dk_04_88 | Dk_04_88_01 | S | 8 | R | 3 | 9 |
| Dk_04_92 | Dk_04_92_02 | I | 5 | I | 6 | 9 |
| Dk_04_92 | Dk_04_92_02 | I | 6 | I | 6 | 9 |
| Dk_04_92 | Dk_04_92_02 | S | 7 | R | 4 | 9 |
| Dk_04_95 | Dk_04_95_04 | I | 5 | I | 6 | 9 |
| Dk_04_95 | Dk_04_95_04 | S | 7 | R | 3 | 8 |
| Dk_04_95 | Dk_04_95_04 | S | 7 | R | 3 | 8 |
| Dk_04_96 | Dk_04_96_04 | R | 4 | I | 5 | 8 |
| Dk_04_96 | Dk_04_96_04 | S | 7 | I | 6 | 9 |
| Dk_04_96 | Dk_04_96_04 | S | 8 | S | 7 | 9 |
| Dk_05_11 | Dk_05_11_03 | R | 3 | R | 4 | 9 |
| Dk_05_11 | Dk_05_11_03 | S | 7 | R | 4 | 9 |
| Dk_05_11 | Dk_05_11_06 | I | 5 | R | 3 | 9 |
| Dk_05_11 | Dk_05_11_06 | I | 5 | R | 4 | 7 |
| Dk_05_14 | Dk_05_14_03 | I | 5 | R | 4 | 5 |
| Dk_05_14 | Dk_05_14_03 | I | 5 | R | 3 | 5 |
| Dk_05_14 | Dk_05_14_03 | S | 8 | I | 5 | 7 |
| Dk_05_14 | Dk_05_14_06 | I | 5 | R | 4 | 9 |
| Dk_05_15 | Dk_05_15_02 | I | 6 | I | 6 | 9 |
| Dk_05_15 | Dk_05_15_02 | I | 6 | R | 4 | 9 |
| Dk_05_15 | Dk_05_15_02 | S | 8 | I | 6 | 9 |
| Dk_05_15 | Dk_05_15_06 | I | 6 | I | 5 | 9 |
| Dk_05_16 | Dk_05_16_02 | I | 5 | I | 5 | 8 |
| Dk_05_16 | Dk_05_16_02 | I | 6 | R | 3 | 9 |
| Dk_05_16 | Dk_05_16_02 | R | 3 | R | 1 | 8 |
| Dk_05_16 | Dk_05_16_02 | I | 6 | I | 6 | 8 |
| Dk_05_24 | Dk_05_24_02 | R | 4 | R | 2 | 6 |
| Dk_05_24 | Dk_05_24_02 | R | 4 | R | 4 | 8 |
| Dk_05_24 | Dk_05_24_02 | I | 5 | I | 5 | 7 |
| Dk_05_24 | Dk_05_24_04 | R | 4 | R | 0 | 7 |
| Dk_05_24 | Dk_05_24_04 | I | 5 | R | 3 | 7 |
| Dk_05_24 | Dk_05_24_04 | R | 4 | R | 3 | 7 |
| Dk_05_24 | Dk_05_24_06 | R | 4 | R | 3 | 7 |
| Dk_05_24 | Dk_05_24_06 | I | 5 | R | 0 | 7 |
| Dk_05_29 | Dk_05_29_02 | R | 3 | R | 2 | 8 |
| Dk_05_29 | Dk_05_29_02 | I | 6 | R | 4 | 8 |
| Dk_05_29 | Dk_05_29_05 | I | 5 | R | 4 | 8 |
| Dk_05_29 | Dk_05_29_05 | I | 5 | I | 6 | 8 |
| Dk_05_30 | Dk_05_30_01 | R | 4 | I | 6 | 8 |
| Dk_05_30 | Dk_05_30_01 | R | 4 | I | 5 | 8 |
| Dk_05_30 | Dk_05_30_01 | I | 6 | R | 0 | 8 |
| Dk_05_30 | Dk_05_30_04 | I | 5 | R | 0 | 8 |
| Dk_05_33 | Dk_05_33_01 | I | 5 | I | 6 | 8 |
| Dk_05_33 | Dk_05_33_01 | I | 5 | R | 4 | 8 |
| Dk_05_33 | Dk_05_33_01 | I | 6 | S | 8 | 8 |
| Dk_05_34 | Dk_05_34_04 | I | 5 | R | 2 | 9 |
| Dk_05_34 | Dk_05_34_04 | I | 6 | R | 4 | 8 |
| Dk_05_34 | Dk_05_34_04 | R | 4 | R | 3 | 8 |
| Dk_05_35 | Dk_05_35_05 | R | 4 | R | 3 | 9 |
| Dk_05_35 | Dk_05_35_05 | I | 5 | S | 8 | 9 |
| Dk_05_35 | Dk_05_35_05 | S | 7 | R | 3 | 8 |
| Dk_05_38 | Dk_05_38_04 | I | 5 | R | 4 | 8 |
| Dk_05_38 | Dk_05_38_04 | I | 6 | I | 5 | 9 |
| Dk_05_38 | Dk_05_38_06 | I | 5 | R | 0 | 8 |
| Dk_05_42 | Dk_05_42_03 | R | 4 | R | 3 | 7 |
| Dk_05_42 | Dk_05_42_03 | R | 4 | R | 4 | 7 |
| Dk_05_42 | Dk_05_42_03 | I | 6 | I | 5 | 7 |
| Dk_05_42 | Dk_05_42_06 | R | 3 | I | 6 | 7 |
| Dk_05_42 | Dk_05_42_06 | I | 6 | I | 6 | 8 |
| Dk_05_44 | Dk_05_44_01 | R | 4 | I | 5 | 7 |
| Dk_05_44 | Dk_05_44_01 | I | 5 | R | 3 | 6 |
| Dk_05_44 | Dk_05_44_01 | I | 5 | I | 5 | 7 |
| Dk_05_47 | Dk_05_47_03 | R | 4 | I | 5 | 8 |
| Dk_05_47 | Dk_05_47_03 | I | 6 | S | 7 | 7 |
| Dk_05_47 | Dk_05_47_03 | S | 8 | I | 5 | 8 |
| Dk_05_47 | Dk_05_47_03 | R | 4 | R | 3 | 7 |
| Dk_05_47 | Dk_05_47_03 | R | 4 | R | 0 | 9 |
| Dk_05_53 | Dk_05_53_07 | R | 4 | R | 2 | 9 |
| Dk_05_53 | Dk_05_53_07 | I | 5 | R | 3 | 9 |
| Dk_05_56 | Dk_05_56_03 | R | 4 | I | 6 | 8 |
| Dk_05_56 | Dk_05_56_03 | S | 7 | R | 3 | 9 |
| Dk_06_01 | Dk_06_01_02 | R | 4 | R | 1 | 8 |
| Dk_06_01 | Dk_06_01_02 | I | 6 | R | 3 | 8 |
| Dk_06_01 | Dk_06_01_02 | S | 7 | I | 5 | 9 |
| Dk_06_01 | Dk_06_01_03 | I | 6 | R | 3 | 9 |
| Dk_06_01 | Dk_06_01_03 | I | 6 | R | 3 | 8 |
| Dk_06_01 | Dk_06_01_03 | S | 7 | R | 2 | 7 |
| Dk_06_01 | Dk_06_01_05 | I | 6 | R | 1 | 7 |
| Dk_06_01 | Dk_06_01_05 | S | 7 | S | 7 | 8 |
| Dk_06_01 | Dk_06_01_05 | S | 8 | S | 7 | 8 |
| Dk_06_03 | Dk_06_03_01 | R | 4 | R | 4 | 9 |
| Dk_06_03 | Dk_06_03_01 | I | 6 | R | 4 | 8 |
| Dk_06_03 | Dk_06_03_01 | I | 6 | R | 3 | 9 |
| Dk_06_03 | Dk_06_03_02 | I | 6 | I | 6 | 8 |
| Dk_06_03 | Dk_06_03_02 | S | 7 | I | 5 | 7 |
| Dk_06_03 | Dk_06_03_02 | S | 8 | I | 6 | 7 |
| Dk_06_04 | Dk_06_04_04 | R | 4 | R | 2 | 7 |
| Dk_06_04 | Dk_06_04_04 | I | 5 | I | 6 | 7 |
| Dk_06_04 | Dk_06_04_04 | I | 6 | R | 1 | 7 |
| Dk_06_04 | Dk_06_04_05 | I | 6 | R | 3 | 8 |
| Dk_06_04 | Dk_06_04_05 | S | 8 | R | 0.5 | 8 |
| Dk_06_04 | Dk_06_04_05 | S | 8 | R | 0 | 8 |
| Dk_06_05 | Dk_06_05_02 | I | 6 | I | 5 | 8 |
| Dk_06_05 | Dk_06_05_02 | S | 7 | S | 7 | 9 |
| Dk_06_05 | Dk_06_05_03 | I | 5 | R | 4 | 9 |
| Dk_06_05 | Dk_06_05_03 | I | 6 | S | 8 | 9 |
| Dk_06_05 | Dk_06_05_03 | I | 6 | I | 6 | 8 |
| Dk_06_06 | Dk_06_06_02 | R | 4 | I | 5 | 8 |
| Dk_06_06 | Dk_06_06_02 | I | 6 | I | 5 | 8 |
| Dk_06_06 | Dk_06_06_02 | S | 7 | S | 7 | 8 |
| Dk_06_52 | Dk_06_52_01 | I | 5 | R | 4 | 9 |
| Dk_06_52 | Dk_06_52_01 | I | 6 | R | 3 | 8 |
| Dk_06_52 | Dk_06_52_01 | R | 3 | R | 4 | 7 |
| Dk_06_52 | Dk_06_52_01 | R | 4 | I | 5 | 8 |
| Dk_06_52 | Dk_06_52_01 | R | 4 | R | 3 | 8 |
| Dk_06_53 | Dk_06_53_01 | I | 5 | I | 6 | 8 |
| Dk_06_53 | Dk_06_53_02 | R | 4 | R | 3 | 7 |
| Dk_06_53 | Dk_06_53_02 | I | 6 | R | 2 | 8 |
| Dk_06_53 | Dk_06_53_03 | I | 5 | R | 4 | 8 |
| Dk_06_53 | Dk_06_53_03 | I | 5 | R | 4 | 8 |
| Dk_06_53 | Dk_06_53_03 | I | 5 | I | 5 | 9 |

TABLE 8
Summary of confirmed NLRs following screening at the T2 with wheat stripe rust.

| | | | Domain | | |
|---|---|---|---|---|---|
| Construct | Species | Accession | Structure | tpm | Status |
| Dk_01_35 | Aegilops sharonensis | 6793 | NB-LRR | 8.81425 | Confirmed |
| Dk_01_55 | Aegilops sharonensis | 6793 | NB-LRR | 8.3177 | Confirmed |
| Dk_01_57 | Aegilops sharonensis | 1998 | NB-LRR | 10.7081 | Confirmed |
| Dk_01_59 | Aegilops sharonensis | 1998 | NB-LRR-CC- | 2.4473 | Confirmed |
| | | | LRR | | |
| Dk_01_60 | Aegilops sharonensis | 1998 | NB-LRR | 4.81691 | Confirmed |
| Dk_01_61 | Cynosurus cristatus | PI251810 | NB-LRR-CC | 3.07035 | Confirmed |
| Dk_01_62 | Cynosurus cristatus | PI251810 | NB-LRR | 20.3939 | Confirmed |
| Dk_01_64 | Cynosurus cristatus | PI251810 | CC-NB-LRR | 0.944396 | Confirmed |
| Dk_01_68 | Cynosurus cristatus | PI642806 | NB-LRR | 0.940361 | Confirmed |
| Dk_02_02 | Koeleria macrantha | PI206274 | CC-NB-LRR | 3.05767 | Confirmed |
| Dk_02_03 | Koeleria macrantha | PI206274 | NB-LRR | 2.20239 | Confirmed |
| Dk_02_06 | Koeleria macrantha | PI206274 | NB-LRR | 0.864765 | Confirmed |
| Dk_02_07 | Koeleria macrantha | PI206274 | NB-LRR | 0.877035 | Confirmed |
| Dk_02_08 | Koeleria macrantha | PI206274 | CC-NB-LRR | 0.891724 | Confirmed |
| Dk_02_11 | Koeleria macrantha | PI206274 | NB-LRR | 0.934462 | Confirmed |
| Dk_02_13 | Koeleria macrantha | PI206274 | CC-NB-LRR | 0.789584 | Confirmed |
| Dk_02_14 | Koeleria macrantha | PI206274 | NB-LRR | 0.919367 | Confirmed |
| Dk_02_19 | Koeleria macrantha | PI440454 | NB-LRR | 0.596913 | Confirmed |
| Dk_02_20 | Koeleria macrantha | PI440454 | CC-NB-LRR | 0.582753 | Confirmed |
| Dk_02_25 | Koeleria macrantha | PI440454 | NB-LRR | 0.68745 | Confirmed |
| Dk_02_34 | Koeleria macrantha | W617985 | NB-LRR | 0.999572 | Confirmed |
| Dk_02_35 | Koeleria macrantha | W617985 | NB-LRR | 1.24163 | Confirmed |
| Dk_02_36 | Koeleria macrantha | W617985 | NB-LRR | 1.39549 | Confirmed |
| Dk_02_38 | Koeleria macrantha | W617985 | NB-LRR | 0.985448 | Confirmed |
| Dk_02_39 | Koeleria macrantha | W617985 | NB-LRR | 0.837721 | Confirmed |
| Dk_02_42 | Koeleria macrantha | W617985 | NB-LRR | 1.21722 | Confirmed |
| Dk_02_44 | Koeleria macrantha | W617985 | other-NB-LRR | 0.975831 | Confirmed |
| Dk_02_46 | Koeleria macrantha | W617985 | NB-LRR | 1.05232 | Confirmed |
| Dk_03_13 | Cynosurus cristatus | PI251810 | NB-LRR | 0.915483 | Confirmed |
| Dk_03_16 | Cynosurus cristatus | PI595080 | NB-LRR | 1.04424 | Confirmed |
| Dk_03_19 | Cynosurus cristatus | PI595080 | CC-NB-LRR- | 2.82661 | Confirmed |
| | | | CC | | |
| Dk_03_48 | Holcus lanatus | PI462334 | NB-LRR | 3.60074 | Confirmed |
| Dk_03_58 | Koeleria macrantha | PI440454 | NB-LRR | 0.536192 | Confirmed |
| Dk_03_60 | Koeleria macrantha | W617985 | CC-NB-LRR- | 0.886655 | Confirmed |
| | | | CC-LRR | | |
| Dk_04_34 | Hordeum vulgare | Golden | NB-LRR | 3.3888 | Confirmed |
| | | Promise | | | |
| Dk_04_44 | Aegilops bicornis | 2319 | NB-LRR | 4.34313 | Confirmed |
| Dk_04_85 | Aegilops bicornis | 6065 | NB-LRR | 17.8233 | Confirmed |
| Dk_04_88 | Aegilops bicornis | 6065 | NB-LRR | 1.88129 | Confirmed |
| Dk_04_92 | Aegilops bicornis | 6065 | CC-NB-LRR- | 1.14481 | Confirmed |
| | | | CC | | |
| Dk_04_95 | Aegilops bicornis | 6065 | CC-NB-LRR | 1.33593 | Confirmed |
| Dk_04_96 | Aegilops bicornis | 6065 | NB-LRR | 3.51359 | Confirmed |
| Dk_05_11 | Aegilops longissima | 1059 | CC-NB-LRR | 1.5598 | Confirmed |
| Dk_05_14 | Aegilops longissima | 1059 | NB-LRR | 4.92436 | Confirmed |
| Dk_05_15 | Aegilops longissima | 1059 | NB-LRR | 1.42073 | Confirmed |
| Dk_05_16 | Aegilops longissima | 1059 | NB-LRR | 6.34735 | Confirmed |
| Dk_05_24 | Aegilops longissima | 1059 | CC-NB-LRR | 2.89535 | Confirmed |
| Dk_05_29 | Aegilops longissima | 1255 | CC-NB-LRR | 2.20762 | Confirmed |
| Dk_05_30 | Aegilops longissima | 1255 | CC-NB-CC- | 1.51375 | Confirmed |
| | | | LRR | | |
| Dk_05_33 | Aegilops longissima | 1255 | NB-LRR | 1.11984 | Confirmed |
| Dk_05_34 | Aegilops longissima | 1255 | NB-LRR | 1.05088 | Confirmed |
| Dk_05_35 | Aegilops longissima | 1255 | NB-LRR-CC- | 8.22726 | Confirmed |
| | | | LRR | | |
| Dk_05_38 | Aegilops longissima | 1255 | CC-NB-LRR | 3.77169 | Confirmed |
| Dk_05_42 | Aegilops longissima | 1285 | NB-LRR | 4.7744 | Confirmed |
| Dk_05_44 | Aegilops longissima | 1285 | NB-LRR | 2.23065 | Confirmed |
| Dk_05_47 | Aegilops longissima | 1509 | NB-LRR | 2.44642 | Confirmed |
| Dk_05_53 | Aegilops longissima | 1509 | CC-NB-LRR | 3.68828 | Confirmed |
| Dk_05_56 | Aegilops longissima | 1509 | NB-LRR | 3.02411 | Confirmed |
| Dk_06_01 | Brachypodium | Bd21 | NB-LRR | 5.44453 | Confirmed |
| | distachyon | | | | |
| Dk_06_03 | Aegilops longissima | 1059 | NB-LRR | 3.65758 | Confirmed |
| Dk_06_04 | Aegilops longissima | 1255 | NB-LRR | 2.53782 | Confirmed |
| Dk_06_05 | Aegilops longissima | 1255 | CC-NB-LRR | 2.78425 | Confirmed |
| Dk_06_06 | Aegilops longissima | 1255 | NB-LRR | 3.48985 | Confirmed |
| Dk_06_52 | Aegilops searsii | 6229 | CC-NB-LRR | 1.27708 | Confirmed |
| Dk_06_53 | Aegilops searsii | 6229 | NB-LRR | 1.75958 | Confirmed |

The article “a” and “an” are used herein to refer to one or more than one (i.e., to at least one) of the grammatical object of the article. By way of example, “an element” means one or more element.

Throughout the specification the word “comprising,” or variations such as “comprises” or “comprising,” will be understood to imply the inclusion of a stated element, integer or step, or group of elements, integers or steps, but not the exclusion of any other element, integer or step, or group of elements, integers or steps.

All publications and patent applications mentioned in the specification are indicative of the level of those skilled in the art to which this invention pertains. All publications and patent applications are herein incorporated by reference to the same extent as if each individual publication or patent application was specifically and individually indicated to be incorporated by reference.

Although the foregoing invention has been described in some detail by way of illustration and example for purposes of clarity of understanding, it will be obvious that certain changes and modifications may be practiced within the scope of the appended claims.

1. A method for preparing a library of candidate plant disease resistance (R) genes against at least one plant pathogen of interest, the method comprising
selecting from each of one or more plants of interest a subpopulation of highly expressed nucleotide-binding leucine rich repeat genes (NLRs) from among a population of constitutively expressed NLRs in an organ or other part of the one or more plants, so as to produce a library of candidate R genes,
wherein a highly expressed NLR comprises a relative expression level in the organ or other part of the plant that is greater than the relative expression levels in the organ or other part of the plant of at least 65% of the constitutively expressed NLRs.

2. The method of claim 1, wherein one or more of the NLRs in the population of constitutively expressed NLRs further comprises at least one feature of interest.

3. The method of claim 1, wherein selecting a subpopulation of highly expressed NLRs further comprises selecting NLRs comprising at least one feature of interest.

4. The method of claim 2, wherein the at least one feature of interest is selected from the group consisting of:
(a) the presence of intraspecific variation in the amino acid sequence encoded by an NLR;
(b) the absence of intraspecific variation in the amino acid sequence encoded by an NLR;
(c) the presence of interspecific variation in the amino acid sequence encoded by an NLR;
(d) the absence of interspecific variation in the amino acid sequence encoded by an NLR; and
(e) substantial intraspecific allelic variation in the amino acid sequence encoded by an NLR.

5-7. (canceled)

8. The method of claim 1, wherein the plant or plants of interest do not support the growth or lifecycle completion of the plant pathogen(s) of interest.

9. The method of claim 1, wherein the organ is selected from the group consisting of a leaf, a root, and a stem.

10-22. (canceled)

23. A library of candidate R genes prepared according to the method of claim 1.

24. A transgenic plant comprising a candidate R gene from the library of claim 23, or a collection of transgenic plants, wherein each of the transgenic plants is transformed with a candidate R gene from the library.

25. (canceled)

26. A method for identifying a plant disease resistance (R) gene against a plant pathogen of interest, the method comprising
(i) producing a transformed plant by transforming a host plant with a candidate R gene selected from the library of claim 23, wherein the host plant is a host for the plant pathogen of interest,
(ii) contacting the transformed plant with the plant pathogen of interest under environmental conditions suitable for the development of disease, and
(iii) determining if the transformed plant displays enhanced resistance to the plant pathogen of interest when compared to a control plant lacking the candidate R gene, wherein the candidate R gene is an R gene against the plant pathogen of interest when the transformed plant displays enhanced resistance to plant disease symptoms caused by the plant pathogen of interest.

27. A method for identifying a plant disease resistance (R) gene against a plant pathogen of interest, the method comprising
(i) contacting a transgenic plant or a collection of transgenic plants according to claim 24 with the plant pathogen of interest under environmental conditions suitable for the development of disease symptoms, wherein a control plant lacking the candidate R gene is a host for the plant pathogen of interest and the plant pathogen is capable of causing plant disease symptoms on the host plant, and
(ii) assessing disease symptoms on the transgenic plant(s), wherein a transgenic plant comprises an R gene against the plant pathogen of interest when the transgenic plant displays enhanced resistance to plant disease caused by the plant pathogen of interest, when compared to a control plant lacking the candidate R gene.

28-32. (canceled)

33. A nucleic acid molecule, an expression cassette comprising the nucleic acid molecule and an operably linked heterologous promoter, a vector comprising the nucleic acid molecule or the expression cassette, or a host cell transformed with the nucleic acid molecule, expression cassette, or vector, wherein the nucleic acid molecule comprises a nucleotide sequence selected from the group consisting of:
(a) the nucleotide sequence set forth in SEQ ID NO: 1, 3, 5, 7, 9, 11, 13, 15, 17, 19, 21, 23, 25, 29, 31, 33, 35, 37, 39, 41, 43, 45, 47, 49, 51, 53, 55, 57, 59, 61, 63, 65, 67, 69, 71, 73, 75, 77, 79, 81, 83, 85, 87, 89, 91, 93, 95, 97, 99, 101, 103, 105, 107, 109, 111, 113, 115, 117, 119, 121, 123, 125, 127, 129, 131, 133, 135, 137, 139, 141, 143, 145, 147, 149, 151, 153, 155, 157, 159, 161, 163, 165, 167, 169, 171, 173, 175, 177, 179, 181, 183, 185, or 187;
(b) a nucleotide sequence encoding a polypeptide comprising an amino acid sequence set forth in SEQ ID NO: 2, 4, 6, 8, 10, 12, 14, 16, 18, 20, 22, 24, 26, 30, 32, 34, 36, 38, 40, 42, 44, 46, 48, 50, 52, 54, 56, 58, 60, 62, 64, 66, 68, 70, 72, 74, 76, 78, 80, 82, 84, 86, 88, 90, 92, 94, 96, 98, 100, 102, 104, 106, 108, 110, 112, 114, 116, 118, 120, 122, 124, 126, 128, 130, 132, 134, 136, 138, 140, 142, 144, 146, 148, 150, 152, 154, 156, 158, 160, 162, 164, 166, 168, 170, 172, 174, 176, 178, 180, 182, 184, 186, or 188,
(c) a nucleotide sequence having at least 75% sequence identity to at least one of the nucleotide sequences set forth in (a), wherein the nucleic acid molecule is capable of conferring to a plant resistance to a plant disease selected from the group consisting of wheat stem rust, wheat stripe rust, wheat leaf rust, wheat blast, and wheat powdery mildew; and
(d) a nucleotide sequence encoding a polypeptide having at least 75% amino acid sequence identity to at least one of the full-length amino acid sequences set forth in (b), wherein the nucleic acid molecule is capable of conferring to a plant resistance to a plant disease selected from the group consisting of wheat stem rust, wheat stripe rust, wheat leaf rust, wheat blast, and wheat powdery mildew.

34-39. (canceled)

40. A transgenic plant or seed comprising stably incorporated in its genome a polynucleotide construct comprising a nucleotide sequence selected from the group consisting of the nucleotide sequences of (a)-(d) of claim 33.

41. A method for producing a plant with enhanced resistance to a plant disease, the method comprising introducing a polynucleotide construct into at least one plant cell, the polynucleotide construct comprising a nucleotide sequence selected from the group consisting of the nucleotide sequences of (a)-(d) of claim 33.

42-46. (canceled)

47. A method of limiting a plant disease in agricultural crop production, the method comprising planting a seed according claim 40, and growing a plant under conditions favorable for the growth and development of the plant.

48. (canceled)

49. A human or animal food product produced using the plant or seed of claim 40.

50. (canceled)

51. A method for preparing a library of candidate plant pest resistance (R) genes against at least one plant pest of interest, the method comprising
selecting from each of one or more plants of interest a subpopulation of highly expressed nucleotide-binding leucine rich repeat genes (NLRs) from among a population of constitutively expressed NLRs in an organ or other part of the one or more plants, so as to produce a library of candidate R genes,
wherein a highly expressed NLR comprises a relative expression level in the organ or other part of the plant that is greater than the relative expression levels in the organ or other part of the plant of at least 65% of the constitutively expressed NLRs.

52. A library of candidate R genes prepared according to the method of claim 51.

53. A transgenic plant comprising a candidate R gene from the library of claim 52, or a collection of transgenic plants, wherein each of the transgenic plants is transformed with at least one candidate R gene from the library.

54. (canceled)

55. A method for identifying a plant pest resistance (R) gene against a plant pest of interest, the method comprising
(i) producing a transformed plant by transforming a host plant with a candidate R gene selected from the library of claim 52, wherein the host plant is a host for the plant pest of interest,
(ii) contacting the transformed plant with the plant pest of interest under environmental conditions suitable for the development of disease symptoms or other damage to the transformed plant, and
(iii) determining if the transformed plant displays enhanced resistance to the plant pest of interest when compared to a control plant lacking the candidate R gene, wherein the candidate R gene is an R gene against the plant pest of interest when the transformed plant displays enhanced resistance to plant disease or other damage caused by the plant pest of interest.

56. A method for identifying a plant pest resistance (R) gene against a plant pest of interest, the method comprising
(i) contacting a transgenic plant or a collection of transgenic plants according to claim 53 with the plant pest of interest under environmental conditions suitable for the development of disease symptoms or other damage, wherein a control plant lacking the candidate R gene is a host for the plant pest of interest and the plant pest is capable of causing disease symptoms or other damage to the host plant, and
(ii) assessing damage on the transgenic plant(s), wherein a transgenic plant comprises an R gene against the plant pest of interest when the transgenic plant displays enhanced resistance to plant disease or other damage caused by the plant pest of interest, when compared to a control plant lacking the candidate R gene.

57-61. (canceled)
